(19) United States
(12) Patent Application Publication
(10) Pub. No.: US 2025/0122458 A1
(43) Pub. Date: Apr. 17, 2025

(54) TRANSMEMBRANE DEVICE

(71) Applicant: CAMBRIDGE ENTERPRISE LIMITED, Cambridge (GB)

(72) Inventors: Róisín Meabh OWENS, Cambridge (GB); Charalampos PITSALIDIS, Thessaloniki (GR); Alexander Jacob BOYS, Cambridge (GB); Chrysanthi-Maria MOYSIDOU, Cambridge (GB); Douglas Carl VAN NIEKERK, Cambridge (GB)

(21) Appl. No.: 18/685,450

(22) PCT Filed: Aug. 24, 2022
(86) PCT No.: PCT/GB2022/052181

§ 371 Date: Feb. 21, 2024

(30) Foreign Application Priority Data
Aug. 25, 2021 (EP) 21386056.2
Sep. 8, 2021 (GB) 2112819.4

(51) Int. Cl. C12M 1/12; C12M 1/34; C12N 5/071; C12N 5/077

(52) U.S. Cl. CPC C12M 25/04; C12M 25/14; C12M 41/00; C12N 5/0656; C12N 5/0679; C12N 5/069; C12N 2513/00; C12N 2537/10

(57) ABSTRACT

The present invention provides A porous scaffold membrane configured for use in a well plate insert, method of making the insert, an apparatus comprising the inset and uses of the insert in monitoring cell culture.

### FIELD OF INVENTION

The invention relates to conducting porous scaffold membranes for 3-dimensional cell culture and devices that incorporate the membranes, as well as methods using such devices. In particular, the invention relates to inserts, including the membranes for use in cell culture wells, such as well plates. The invention also relates to apparatus for dynamically monitoring properties, such as electrical properties, of 3-dimensional cell cultures seeded onto and within the porous scaffolds membranes and methods of monitoring the 3-dimensional cell cultures.

### BACKGROUND

A widely used cell culture setup for various in vitro applications (e.g., cell migration, toxicology, drug/nutrient transport) is the Transwell® insert, where, typically, cells are grown on filter membranes suspended in culture media. This format also facilitates compartmentalisation of the culture (apical-basal compartments), essential for developing epithelial and endothelial tissues in vitro,^{1,2} as well as various types of co-cultures (e.g., cancer-immune cell interaction,³ host-microbe interactions⁴).

Assessment of the barrier function or integrity of such co-cultures and tissues is typically done via permeability assays using tracer molecules. For real time evaluation, electrical measurement of Trans-Epi/Endothelial Electrical Resistance (TEER) may be used. Electrical monitoring provides a rapid and non-destructive assessment of cell health and state, following in real time the evolution of the tissue growth and function. The most common techniques for measuring TEER involve either single-frequency measurements using ‘chopstick electrodes’ (e.g., EVOM) or electrical impedance spectroscopy (EIS), which offers a more detailed characterisation of cellular health and status, due to current measurements in a broad frequency spectrum.

Impedance-based cell monitoring is a highly sensitive method for monitoring cell growth, proliferation and differentiation. However, the sensitivity of this method is dependent on whether the cells are directly in contact with the electrode or suspended between electrodes, as is the case with cells grown on Transwell inserts. When not in contact with the electrode, TEER measurements are typically only possible with cell layers that are characterised by high levels of resistance, typically >30 Ohm·cm². On the other hand, TEER cannot be measured on cells attached to an electrode, as the 2D environment and the absence of the apical/basal compartments, does not provide cells with the necessary cues to differentiate towards the desired tissue phenotype: polarised, intact and selectively permeable epi-/endo-thelial barriers. This planar culture configuration also impedes transport of nutrients and other molecules (e.g., potential drug compounds) across the layers, thus prohibiting studies looking at such mechanisms.

It has previously been shown that conducting polymer (CP) scaffolds can be used as substrates for 3D cell cultures, as well as active elements in electronic transducers for label-free and real-time cell sensing. The porous nature of the CP scaffolds, allows for flow of nutrients/compounds into and out of the scaffold, creating a highly favourable environment for cell growth. As cells attach, proliferate and differentiate within the porous network, they may be monitored by a CP electrode.

An example of such CP scaffolds include the tube based system disclosed in “Pitsalidis, Charalampos, et al. “Transistor in a tube: A route to three-dimensional bioelectronics.” Science advances 4.10 (2018): eaat4253.” and “Moysidou, Chrysanthi-Maria, et al. “3D Bioelectronic Model of the Human Intestine.” Advanced Biology 5.2 (2021): 2000306.”. Both of which disclose the use of a poly(3,4-ethylenedioxythiophene) doped with poly(styrene sulfonate) (PEDOT:PSS) for making a tube based system that mimics intestinal organoids.

However, these devices are designed for continuous flow experiments and suffer from lack of throughput which is problematic for routine biological assays.

### SUMMARY OF INVENTION

It is an aim of the present invention to provide a conducting polymer scaffold for growth and maintenance of 3-dimensional cell cultures configured for use in high throughput methods and devices.

It is an aim of the present invention to provide devices and methods for high throughput electrical interrogation of cells.

It is an aim of the present invention to provide improved devices and methods for drug discovery, efficacy and/or toxicity studies.

Provided in a first aspect of the invention is a porous scaffold membrane configured for use in a well plate insert comprising a conducting polymer wherein the porous scaffold membrane comprises a porosity of at least about 30% and an average pore diameter from about 10 μm to about 150 μm. The porosity and/or average pore diameter may help to allow cells cultured in and/or on the porous scaffold membrane to attach to the porous scaffold membrane as well as allow for the transport of molecules, such as nutrients and gases through the membrane and any cells cultured therein and/or therein. Thus, allowing for cell cultures to be maintained in and/or on the porous scaffold membrane.

In some embodiments, the conducting polymer comprises poly(3,4-ethylenedioxythiophene) polystyrene sulfonate (PEDOT:PSS).

In some embodiments, the porous scaffold membrane is configured to allow gas and nutrient permeation.

In some embodiments, the porous scaffold membrane comprises a thickness from about 100 μm to 1000 μm. For example, 100 μm, 150 μm, 200 μm, 250 μm, 300 μm, 350 μm, 400 μm, 450 μm, 500 μm. 550 μm, 600 μm, 650 μm, 700 μm, 750 μm, 800 μm, 850 μm, 900 μm, 950 μm, or 1000 μm.

In some embodiments, the porous scaffold membrane comprises a conductivity from 5 to 100 S/cm.

In some embodiments, the porous scaffold membrane comprises an impedance in the presence of phosphate buffered saline of between 1 and 500 Ohm at 1 kHZ and a membrane thickness of 500 μm. In some embodiments, at least 40 Ohms at 1 kHZ and a membrane thickness of 500 μm. For example, between 40 and 500 Ohm at 1 kHZ and a membrane thickness of 500 μm.

In some embodiments, the porous scaffold membrane comprises a macroporous morphology.

In some embodiments, the porous scaffold membrane comprises a continuous structure. In some embodiments, the porous scaffold membrane comprises a disc shape. In some embodiments, the porous scaffold membrane comprises a shape that corresponds to an insert for support device as described herein.

In some embodiments, the porous scaffold membrane further comprises at least one additional agent selected from one or more of:
a. at least one cryoprotectant;
b. at least one oligosaccharide;
c. at least one polymer;
d. at least one conductivity enhancing agent;
e. at least one biopolymer; and/or
f. at least one crosslinking agent.

In some embodiments, the porous scaffold membrane further comprises at least one additional agent.

In some embodiments, the at least one additional agent comprises at least one cryoprotectant.

In some embodiments, the at least one additional agent comprises at least one oligosaccharide.

In some embodiments, the at least one additional agent comprises at least one polymer.

In some embodiments, the at least one additional agent comprises at least one conductivity enhancing agent.

In some embodiments, the at least one additional agent comprises at least one biopolymer.

In some embodiments, the at least one additional agent comprises at least one crosslinking agent.

In another aspect of the invention there is provided a method of producing a porous scaffold membrane as described herein, the method comprising:
a. forming an aqueous dispersion of a conducting polymer and poly(styrene sulfonate);
b. adding a crosslinking agent to the aqueous dispersion; and
c. lyophilising the aqueous solution.

In some embodiments, the method further comprises prior to step c. pouring the aqueous dispersion into a mould. For example, into a mould that is configured to provide a porous scaffold membrane with a shape that is configured for use in a well plate insert. For example, a mould for providing a disc shaped porous scaffold membrane.

In some embodiments, the method further comprises prior to step c., adding one or more of:
a. at least one cryoprotectant;
b. at least one oligosaccharide;
c. at least one polymer;
d. at least one conductivity enhancing agent;
e. at least one biopolymer; and/or
f. at least one further crosslinking agent, to the aqueous dispersion.

In some embodiments, the method further comprises prior to step c., adding at least one cryoprotectant.

In some embodiments, the method further comprises prior to step c., adding at least one oligosaccharide.

In some embodiments, the method further comprises prior to step c., adding at least one polymer.

In some embodiments, the method further comprises prior to step c., adding at least one conductivity enhancing agent.

In some embodiments, the method further comprises prior to step c., adding at least one biopolymer.

In some embodiments, the method further comprises prior to step c., adding at least one further crosslinking agent.

In another aspect of the invention there is provided a method of forming a 3-dimensional cell culture comprising the steps of:
a. seeding a porous scaffold membrane as described herein with a first cell type; wherein the first cell type is disposed within the porous scaffold membrane;
b. seeding at least one second cell type to at least one surface of the porous scaffold membrane; and
c. culturing the first and at least one second cells types.

In some embodiments, the first cell type comprises connective tissue cells and/or stroma cells; optionally wherein the first cell type comprises one or more of fibroblasts, neuronal cells and/or immune cells.

In some embodiments, the first cell type comprises connective tissue cells. In some embodiments, the first cell type comprises stroma cells. In some embodiments, the first cell type comprises connective tissue cells and stroma cells. In some embodiments, the first cell type comprises fibroblasts. In some embodiments, the first cell type comprises neuronal cells. In some embodiments, the first cell type comprises immune cells. In some embodiments, the first cell type comprises fibroblasts and neuronal cells. In some embodiments, the first cell type comprises fibroblasts and immune cells. In some embodiments, the first cell type comprises neuronal cells and immune cells. In some embodiments, the first cell type comprises fibroblasts, neuronal cells and immune cells.

In some embodiments, the at least one second cell type comprises epithelial and/or endothelial cells.

In some embodiments, the at least one second cell type comprises epithelial cells. In some embodiments, the at least one second cell type comprises endothelial cells. In some embodiments, the at least one second cell type comprises epithelial and endothelial cells.

In another aspect of the invention there is provided a 3-dimensional cell culture obtainable by the method of forming a 3-dimensional cell culture as described herein.

In another aspect of the invention there is provided an insert for dynamically monitoring a 3-dimensional cell culture comprising;
a. a porous scaffold membrane as described herein;
b. at least one working electrode; and
c. a body configured to be disposed in a support a device;
wherein the at least one working electrode is in electrical contact with the porous scaffold membrane.

In some embodiments, the at least one working electrode comprises a metal. In some embodiments, the metal comprises gold, platinum, silver and/or lead. In some embodiments, the metal comprises gold. In some embodiments, the metal comprises platinum. In some embodiments, the metal comprises silver. In some embodiments, the metal comprises lead.

In some embodiments, the at least one working electrode comprises an aperture.

The aperture allows for a portion of the porous scaffold membrane to be exposed to cell culture media when in use. Thus allowing for cells to be maintained on the porous scaffold membrane when in use.

In some embodiments, the insert comprises a first and a second working electrode positioned on opposing surfaces of the porous scaffold membrane. For example, one working electrode located on an upper surface of the porous scaffold membrane and one working electrode located on a lower surface of the porous scaffold membrane.

In some embodiments, the insert further comprises at least one membrane support member. The membrane support member may help maintain the porous scaffold membrane within the insert.

In some embodiments, the insert further comprises a counter electrode configured to be in electrical contact with the porous scaffold membrane and at least one working electrode via an electrolyte when in use.

In some embodiments, the counter electrode comprises a platinum or stainless steel mesh. The use of a mesh provides a high surface area that may allow for improved electrical monitoring of 3D-cell cultures cultured on and/or in a porous scaffold membrane.

In some embodiments, the counter electrode comprises a porous scaffold membrane as described herein and the counter electrode is isolated from the seeded porous scaffold membrane.

In some embodiments, the counter electrode is integrated with a lid configured to cover the insert.

Provided in another aspect of the invention is an apparatus for dynamically monitoring a 3-dimensional cell culture comprising:
i.) an insert as described herein not including a counter electrode and a counter electrode configured to be in electrical contact with the porous scaffold membrane and at least one working electrode via an electrolyte when in use; or
   ii.) an insert as described herein comprising a counter electrode configured to be in electrical contact with the porous scaffold membrane via an electrolyte when in use; and
a support device configured to receive the insert therein.

In another aspect of the invention there is provided an apparatus for dynamically monitoring a 3-dimensional cell culture comprising:
an insert as described herein;
a counter electrode configured to be in electrical contact with the porous scaffold membrane and at least one working electrode via an electrolyte when in use; and
a support device configured to receive the insert therein.

In another aspect of the invention there is provided an apparatus for dynamically monitoring a 3-dimensional cell culture comprising:
an insert as described herein comprising a counter electrode; and
a support device configured to receive the insert therein.

In some embodiments, the counter electrode is positioned in an upper portion of the support device. For example, in an apical portion of a well of a well plate as defined by an insert disposed therein.

In some embodiments, the insert is an insert that does not include a counter electrode and the counter electrode is positioned in a lower portion of the support device and the at least one working electrode is located on a lower surface of the porous scaffold membrane.

In some embodiments, the support device further comprises a lid configured to cover the insert.

In some embodiments, the counter electrode is integrated with the lid.

In some embodiments, the counter electrode comprises a platinum or stainless steel mesh. The use of a mesh provides a high surface area that allows for improved electrical monitoring of 3D-cell cultures cultured on and/or in a porous scaffold membrane.

In some embodiments, the counter electrode comprises a porous scaffold membrane as described herein.

In some embodiments, the counter electrode comprises a polarisable material. In some embodiments, the counter electrode comprises a non-faradaic material. In some embodiments the counter electrode comprises a material that is non-toxic to cells. In some embodiments, the counter electrode comprises steel. In some embodiments the counter electrode comprises a steel mesh. It has been found stainless steel may act as a polarizable material (i.e. inert) at voltages of interest for the systems and apparatus described herein. Stainless steel may allow for much larger meshes to be used in comparison to platinum, which may improve measurement sensitivity and/or allow for earlier detection of cell barrier formation on the scaffold. Furthermore, Stainless steel can be soldered to standard lead wire by using a phosphoric acid-based flux to yield a highly stable, electrically low impedance contact, which mitigates risk of device failure and measurement contamination by contact impedance. In addition, the use of non-toxic materials may provide for less contamination from the counter electrode, for example, of the cells cultured on the scaffold.

In some embodiments, the apparatus further comprises a reference electrode.

In some embodiments the reference electrode comprises a shapeable or malleable material. In some embodiments, the reference electrode comprises a polarisable material. In some embodiments, the reference electrode comprises a non-faradaic material. In some embodiments, the reference electrode comprises steel. In some embodiments the reference electrode comprises a stainless steel ring. In some embodiments, the reference electrode comprises a quasi-electrode (e.g., a quasi-reference or pseud-reference electrode). The use of a reference electrode may provide increased temporal sensitivity, fine details of cell barrier formation and/or sensitivity (such as absolute sensitivity) of a system or device in comparison to a system not including a reference electrode.

In some embodiments, the support device is a well plate comprising at least one well configured to receive the insert.

In another aspect of the invention there is provided a method of dynamically monitoring a 3-dimensional cell culture comprising the steps of:
a. providing an apparatus as described herein;
b. forming a 3-dimensional cell culture using a method as described herein;
c. disposing the insert into the support device;
d. immersing at least a portion of the 3-dimensional cell culture, the at least one working electrode and the counter electrode in an electrolyte;
e. applying an electrical signal to the 3-dimensional cell culture via the counter electrode and at least one working electrode; and
f. monitoring the electrical properties of the 3-dimensional cell culture and/or the porous scaffold membrane.

In some embodiments, the electrolyte comprises culture medium.

In some embodiments, the monitoring comprises electrical impedance spectroscopy (EIS).

In some embodiments, wherein the insert comprises a first and a second working electrode positioned on opposing surfaces of the porous scaffold membrane, the method comprises operating the insert as a transistor.

In some embodiments, monitoring comprises single frequency transistor-based measurements.

In another aspect of the invention there is provided a kit of parts comprising: an insert not including a counter electrode, a support device as described herein and a counter electrode as described herein.

In another aspect of the invention there is provided a kit of parts comprising: an insert including a counter electrode and a support device as described herein not comprising a counter electrode.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF FIGURES

FIG. 1 shows, (A) scaffolds with isotropic pore structure and (B) shows scaffolds with fan-like and circular macroscopic patterning in the pore structure (anisotropic pore structure);

FIG. 2 shows comparative optical micrographs showing top and side view of PEDOT:PSS porous scaffold membranes with various thicknesses (200, 300, 400 μm);

FIG. 3 shows the effects of various cryoprotectants on pore size of porous scaffold membranes;

FIG. 4 shows the effects of additives, such as oligosaccharides (maltose and cellobiose) and polymers (PVA) on pore morphology of porous scaffold membranes. Before mixing with PEDOT:PSS, the additives were diluted in water as follows: cellobiose (4%), Maltose (2%), PVA (4%);

FIG. 5 shows the effects of additives, such as oligosaccharides (maltose and cellobiose) and polymers (PVA) on the pore size and electrical properties (conductance) of porous scaffold membranes. Before mixing with PEDOT:PSS in 1:2 and 2:1 (v/v) ratio, the additives were diluted in water as follows: cellobiose (4% wt), Maltose (2% wt), PVA (4% wt);

FIG. 6 shows an example of an insert for holding a porous scaffold membrane;

FIG. 7 shows an example of the parts of an insert for holding a porous scaffold membrane;

FIG. 8 shows an example of an insert with an electrode configuration (having a single working electrode positioned above or below the porous scaffold membrane);

FIG. 9 shows an example of a lid (for the insert or support device) with an integrated counter electrode;

FIG. 10 shows a plan view of an example of a lid (for the insert or support device) with an integrated counter electrode;

FIG. 11 shows an example of an insert with a transistor configuration (having a one working electrode positioned on an upper surface of the porous scaffold membrane and one working electrode positioned on a lower surface of the porous scaffold membrane);

FIG. 12 shows an example of an insert for use in studying an air-liquid interface (such as respiratory tissue models) set up with a counter electrode positioned below the insert in a basal (lower) portion of a support device such as a well of a well plate;

FIG. 13 shows (A) a general set up of an insert for use in studying a model of a neurovascular system (such as the blood brain barrier). (B) Shows an example of a device for studying the blood brain barrier in a non-contact model, having a microelectrode array on the bottom of a well with neurons cultured thereon, astrocytes cultured within the porous scaffold membrane and capillary endothelial cells cultured on the upper surface of the porous scaffold membrane. (C) Shows an example of a device for studying the blood brain barrier in a contact model, having a microelectrode array attached to a lower surface of the porous scaffold membrane with neurons cultured thereon, astrocytes cultured within the porous scaffold membrane and capillary endothelial cells cultured on the upper surface of the porous scaffold membrane;

FIG. 14 shows low magnification confocal microscope image of a [TIF (telomerase immortalized fibroblasts)/HUVEC (human umbilical vein endothelial cells)] co-cultured porous scaffold membrane, showing two distinct areas; uncultured scaffold under the gold O-ring electrode (dark blue) and cultured (green/purple). The illustration below shows the 3D organization of cells. The first cell type (e.g. fibroblasts) infiltrate the porous scaffold membrane and the second cell type (e.g. endo-/epithelial cells) form a layer on the surface(s) of the porous scaffold membrane;

FIG. 15 shows a schematic of a 3-dimensional cell cultured porous scaffold membrane when within an insert and disposed in an electrolyte and the corresponding equivalent circuit with 3 elements in series accounting for; the electrolyte (Rs), porous scaffold membrane (CPE) and the cell layer (Cm//Rm);

FIG. 16 shows an example method of folding a mesh counter electrode;

FIG. 17 shows an example lyophilization programme used for freeze drying a porous scaffold membrane;

FIG. 18 shows a schematic diagram and photograph of an insert;

FIG. 19 shows (A) an optical micrograph of a 100 μm-thick porous scaffold membrane and (B) SEM image showing the corresponding pore morphology;

FIG. 20 shows the fabrication steps for the development of an insert and support device;

FIG. 21 shows (A) comparative optical micrographs showing top and side view of PEDOT:PSS porous scaffold membrane with various thicknesses (200, 300, 400 μm). (B) shows photographs showing the ability of a 200 μm porous scaffold membrane to recover its shape after mechanical deformation, indicative of high elasticity;

FIG. 22 shows (A) bode impedance plots measured from multiple devices (thickness: 400 μm, N=6, taken from 2 different scaffold batches). (B) Impedance magnitude |Z| and phase (see inset) spectra measured from porous scaffold membranes of various thickness;

FIG. 23 illustrates the stability of a device for monitoring a 3-dimensional cell culture showing impedance (bode plots) spectra after 14 days in cell culture media from one representative device over time;

FIG. 24 shows confocal microscopy images of (a) an immunostained intestinal epithelial layer on the apical domain of a porous scaffold membrane in use in a device, illustrating the typical chicken-like pattern of ZO-1 protein distribution (in green) in the tight junction network, overlaid by MUC2 (in red) and (b) of the apical brush border of the cell layer and actin filaments of cell cytoskeleton (in red). (c) Magnified y/z (top) and x/z (bottom) orthogonal views of panel b, offering a closer look at the polarisation of the intestinal layer and its characteristic microvilli, rich in actin filaments (in red; scale bars 10 μm). In all cases cells were counterstained for nuclei (in blue);

FIG. 25 shows (A) Confocal microscopy images of TIF LifeAct cells (actin cytoskeleton tagged with Red Fluorescent Protein), counterstained for nuclei (in blue) demonstrating cell infiltration and formation of the characteristic fibrillar network. (B) Immunofluorescence image of the intestinal epithelial layer, illustrating the distribution of ZO-1 protein (in green) in a typical chicken-wire pattern, revealing the formation of the tight junction network of the layer, and the secretion of mucin proteins (MUC2 in red) overlaying the epithelium. Cells were counterstained for nuclei (in blue). (C) Immunofluorescence image of vascular layer on 500 μm thick porous membrane scaffold, showing adherens junctions (VE-cadherin in green) between endothelial cells. Cells were counterstained for nuclei (in blue) and are visible over an underlying layer of fibroblasts (actin in fibroblasts in orange);

FIG. 26 shows impedance (bode plots) spectra (a,d) and immunofluorescence images (b,c,e) of endothelial (HUVEC) cell cultures. (a-c) Shows confluent HUVEC layer as corroborated from the impedance and optical data (d,e) “Patchy” layer showing almost unchanged impedance spectra and HUVEC patches in the optical imaging;

FIG. 27 shows impedance (Bode plots) spectra and phase (inset) of (A) TIF cultured porous scaffold membranes showing the initial (before culture) and after 4 days of culture, (B) shows IEC culture porous scaffold membranes seeded onto the TIF cultured scaffolds at day 1 (where day 1 refers to the first day of IEC culture but is actually day 5 of the overall experiment) and day 21, and (C) HUVEC cells seeded onto TIF cultured scaffolds at day 1 (where day 1 refers to the first day of HUVEC culture but is actually day 5 of the overall experiment) and day 14;.

FIG. 28 shows impedance (bode plots) spectra of TIF cultured porous scaffold membrane showing the initial (before culture) and after 4 days of culture of 4 different devices;

FIG. 29 shows the extracted Rm values over time using the equivalent circuit modelling, corresponding to the TEER values of the epithelial (Caco-2/HT29-MTX) cell monolayer (vs baseline of TIF cultured scaffold after 4 days). Immunofluorescence and % change in TEER of the 3D bioelectronic epithelial model upon exposure to EGTA;

FIG. 30 shows (A) ZO-1 tight junction protein (in green), mucin (MUC2 in red) and (B) filamentous actin (in red), counterstained for cell nuclei (in blue). The partial recovery of the barrier due to the effects of EGTA are clearly shown in both cases, with domains of the epithelium exhibiting adequate resealing of the junctional space (A1, B1) and other domains where cells are rounded, detached from their neighbours, rounded with tight junctions co-localised in their periphery and their filamentous actin relatively disassembled (A2, B2);

FIG. 31 shows impedance spectra (bode plot) of the 3D bioelectronic model, at different time points, upon disruption of the epithelial model with EGTA and after its recovery following washing with fresh media. Inset is the magnification of the bode plot covering the 10-1 k Hz frequency range;

FIG. 32 shows evolution of % TEER change as calculated from the impedance spectra during EGTA exposure and after recovery in fresh media;

FIG. 33 illustrates using a device in a transistor configuration. (A) shows output and (B) transfer transistor characteristics measured for a 400-um porous scaffold membrane and (C) the corresponding transconductance (gm) vs V_{GS} plot. (D) shows pulsed measurements of the porous scaffold membrane based transistors. A V_{GS} of 0.3V was applied for 10 sec with an interval time of 10 sec between pulses at V_{DS} of −0.2V. The device channel/thickness was 400 μm.

FIG. 34 shows the standard configuration and working principle of the transmembrane device;

FIG. 35 shows a schematic illustrating the two electrode (conventional) and the three electrode (including reference electrode) configurations of a system of the device (e.g. insert) of the invention;

FIG. 36 shows a schematic illustrating the general design of a potentiostat wherein the reference electrode forms a negative feedback loop used to correct for the potential drop across the counter electrode interface, such that the potential difference across the working electrode interface is equal to the required stimulus potential;

FIG. 37 shows a finite Element simulation of the potential field instantiated in a simplified system analogous to a transmembrane device (e.g. insert) of the invention;

FIG. 38 shows a depiction of a transmembrane device (e.g. insert of the invention) well incorporating the reference electrode ring;

FIG. 39 shows a bode plot showing the comparison between two pristine devices, one with a Platinum Apical electrode and the other with a stainless steel apical electrode;

FIG. 40 shows a bode plot (a) and Nyquist plot (b) comparing the impedance of a strip of Platinum mesh in PBS using either a true Silver/Silver Chloride reference electrode or a Stainless Steel Ring as the reference electrode. A second strip of Platinum mesh was used as the counter electrode;

FIG. 41 shows Bode plot derived impedance magnitudes of a well in a Transwell plate (considered representative) as measured using the two and three electrode configurations over 19 days while culturing the Fibroblast/MDCK II barrier epithelial model;

FIG. 42 shows a cross-section through the impedance magnitude data, taken from three devices, in both configurations, at 15 Hz, averaged and plotted as a function of time. Fibroblasts were seeded at Day 0 and MDCK II cells were seeded at Day 5. The data points are averages over three devices and the shaded regions indicate the standard deviation;

FIG. 43 shows (A) Confocal microscopy images of TIF LifeAct cells (actin cytoskeleton tagged with Red Fluorescent Protein), counterstained for nuclei (in blue) demonstrating cell infiltration and formation of the characteristic fibrillar network. (21 days TIF monoculture) (B) Immunofluorescence image of the intestinal epithelial layer (Caco-2/HT29-MTX, 21 days) over a fibroblast-cultivated e-transmembrane, illustrating the distribution of ZO-1 protein (in green) in a typical chicken-wire pattern, revealing the formation of the tight junction network of the layer, and the secretion of mucin proteins (MUC2 in red) overlaying the epithelium. Cells were counterstained for nuclei (in blue). (C) Immunofluorescence image of HUVECs (14 days) over a fibroblast-cultivated e-transmembrane. Images show adherens junctions (VE-cadherin in green). Cells were counterstained for nuclei (blue) visible over a layer of fibroblasts (actin in red). (A 500 μm e-transmembrane was used for the HUVEC experiments);

FIG. 44 shows impedance (Bode plots) spectra and the corresponding phase graphs (insets) of (A) TIF cultured scaffold membranes showing the “initial” (refers to uncultured membranes) and after 5 days of culture measurement, (B) IEC cultured scaffold membranes (on Day 21) onto the TIF cultured scaffolds (where “initial” refers to “Day 5” of TIF culture—before seeding IEC), and (C) HUVEC (on Day 14) onto TIF cultured scaffolds (where “initial” refers to “Day 5” of TIF culture—before seeding the IEC); The grey band represents the impedance spectra from (N=3) different devices and the dashed line in each graph is the extracted averaging curve; and

FIG. 45 shows TEER evolution of the epithelial (Caco-2/HT29-MTX) cultured porous scaffold membrane. Data were generated from 4<N<8 different devices and 4 different experiments. 4<N<8 means that for the different timepoints the TEER average and deviation values were calculated from at least N=4 and max 8 devices

### DETAILED DESCRIPTION

Provided herein are porous scaffold membranes. Porous scaffold membranes may be solid or semi-solid substrates having openings or apertures (pores) which allow cells to partially or completely infiltrate the scaffold. Porous scaffold membranes may also allow the growth of cells on the surface of the scaffold. Porous scaffold membranes may allow for the transport of nutrients, gases and other molecules such as signalling molecules and ions through and into the scaffold and ergo through and to the cells cultured on or in the scaffold. Thus the porous scaffold membranes described herein act as a 3-dimensional matrix that allow for the culture and maintenance of cells in a 3-dimensional architecture. Examples of porous scaffold membranes include a fibrous scaffold with openings or apertures between the fibres of the substrate, a mesh with openings or apertures between the network of structural components that make up the mesh, or a solid substrate with holes throughout the substrate, such as a sponge or foam.

The porosity of the porous scaffold membranes described herein may be at least 30%. For example, the porous scaffold membranes may have a porosity of at least 30%, at least 35%, at least 40%, at least 45%, at least 50%, at least 55%, at least 60%, at least 65%, at least 70%, at least 75%, at least 80%, at least 85%, at least 90% or at least 95%. In particular examples the porosity of the scaffold may be 78%.

As mentioned above, the porosity of the scaffold allows for the permeation of nutrients and gases in to and through scaffold. This allows for the exchange of nutrients and gases from cells allowing cells to be cultured and maintained on the scaffold.

Porosity is a measure of the void spaces in a material, and is measured as a fraction, between 0-1, or as a percentage between 0-100%. Porosity may be measured by any standard methods known in the art. For example, by optical based methods such as scanning electron microscopy (SEM), atomic force microscopy (AFM) and/or transmission electron microscopy (TEM). Optical methods in general determine porosity via direct observations of thin cross-sections of the material. Such methods determine the ratio between the area of pores and the total area of the sample analysed.

Another method of determining porosity is computed tomography (CT) based methods. CT based methods use cross-sectional images of an object reconstructed by collecting and integrating a plurality of x-ray projection data from different projection angles to provide information on voids (pores) in a material.

Other methods for determining porosity include imbibition methods, mercury injection methods, and gas expansion methods.

In particular examples, the porosity of the porous scaffold membranes described herein is a porosity as determined by optical methods. In particular, as determined by SEM. For example, the porous scaffold membranes described herein may have a porosity of at least 30% as determined by SEM image analysis.

The porous scaffold membranes provided herein may have an average pore diameter (sometimes referred to as average pore size) from 10 μm to 150 μm. In particular examples, the average pore size may be from 20 μm to 100 μm. In particular examples, the average pore size may be from 22 μm to 80 μm. In particular examples, the average pore size may be from 40 μm to 70 μm. In particular examples, the average pore size may be from 45 μm to 65 μm. In particular examples, the average pore size may be 57 μm. For example, the average pore diameter may be 10 μm, 20 μm, 30 μm, 40 μm, 50 μm, 60 μm, 70 μm, 80 μm, 90 μm, 100 μm, 110 μm, 120 μm, 130 μm, 140 μm or 150 μm.

Average pore size may be determined using optical methods such as scanning electron microscopy (SEM), atomic force microscopy (AFM), computed tomography methods and/or transmission electron microscopy (TEM). Other methods that may be used include X-ray refraction methods, imbibition methods, mercury injection methods, and gas expansion methods.

The average pore size and porosity (or density of pores) may affect the penetration of cells into the scaffold and define the spatial distribution of cells within the 3D matrix of the scaffold. In addition, average pore size and porosity may affect the flow resistance, the transportation of nutrients, and the excretion of waste products from cells cultured thereon and/or therein.

The porous scaffold membranes provided herein may have open cell pores. The pores may be connected to each other thus providing a porous matrix with a 3D network of interconnected pores. The porous scaffold membranes described herein may have a high surface area provided by the 3-dimensional porous structure.

The porous scaffold membranes provided herein may be considered to have a macroporous structure. Macroporous structures have pores in a range of 10 to 1000 μm.

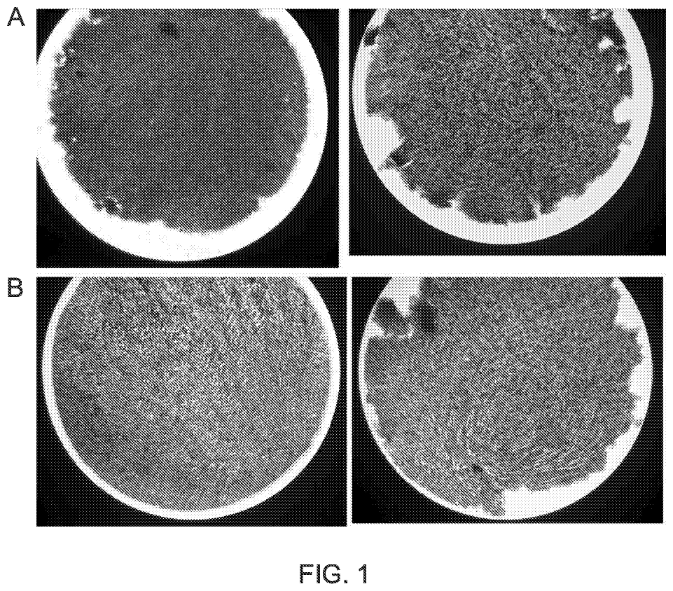

The porous scaffold membranes provided herein may have an isotropic pore morphology. Isotropy refers to uniformity in all orientations. As such, isotropic pore morphology refers to pores that have similar, if not the same, shape, diameter and/or structure throughout a porous material when analysed in any direction through the material. Examples of porous scaffold membranes with isotropic pore structure are shown in FIG. 1A. The porous scaffold membranes provided herein may have an anisotropic pore morphology. Anisotropic materials are materials exhibiting properties with different values when measured in different directions. Examples of porous scaffold membranes with isotropic pore structure are shown in FIG. 1B.

The porous scaffold membranes described herein are referred to as membranes. The term membrane refers to a relatively thin sheet of continuous material. The thickness of the material may be relatively small in comparison to the other dimensions (such as diameter, width or length) of the material.

The porous scaffold membranes described herein have a continuous structure. That is to say that the porous scaffold membranes do not contain any apertures that extend through the membrane but are provided as a single sheet or body of material. The porous scaffold membranes provided herein are configured for use in a well plate. As used herein, a “well plate” is a device used to culture biological cells. A well plate can include a plurality of wells (e.g., 2, 6, 12, 24, 96, 384, or any number of wells therebetween). A well plate may include a cover or a lid which is suitable for covering one or more wells of the well plate. A well plate generally comprises a plastic tray having an upper surface and a lower surface and a plurality of wells extending from the lower surface of the tray. Examples of well plates include Costar® Multiple Well Cell Culture Plates available from Corning Inc., Nunc™ Non-Treated Multidishes and Eppendorf Cell Culture Plates.

Well plates may include an insert that can be placed inside a well to divide a well into two chambers, an apical (upper) portion and a basal (lower) portion. The insert may include a membrane onto which cells can seeded and be cultured, such as the porous scaffold membranes as described herein. The insert may be inserted into a well containing cell culture medium. The insert may include a top compartment having a wall and a porous scaffold membrane as described herein that forms the base of the apical portion. The porous scaffold membrane is held, or suspended in the well, by the insert with at least one surface in contact with the cell culture medium in the well. Examples of inserts that may be modified to include a porous scaffold membrane as described herein include Nunc™ Polycarbonate Cell Culture Inserts, Transwell® inserts, Snapwell™ inserts, Netwell® inserts, and Falcon® inserts available from Corning Inc., Millicell® Cell Culture Inserts available from Merck & Co., THINCERT® CELL CULTURE INSERTS from Greiner and CellCrown™ from Scaffdex Oy.

As such, the porous scaffold membranes provided herein may have a shape and/or dimensions selected to fit within a well or fit into an insert. For example, for a circular well and/or insert the porous scaffold membrane may have a circular shape. This may allow the scaffold to be incorporated into the insert. As such, the porous scaffold membrane may be disc shaped. In another example, the well and/or insert may be a square and as such the porous scaffold membrane may have a square shape. The porous scaffold membranes may be continuous, so that when suspended in a well by an insert, the porous scaffold membrane acts a barrier or separator defining a lower portion of the well (below the porous membrane scaffold) and an upper portion of the well (above the porous scaffold membrane) as described above.

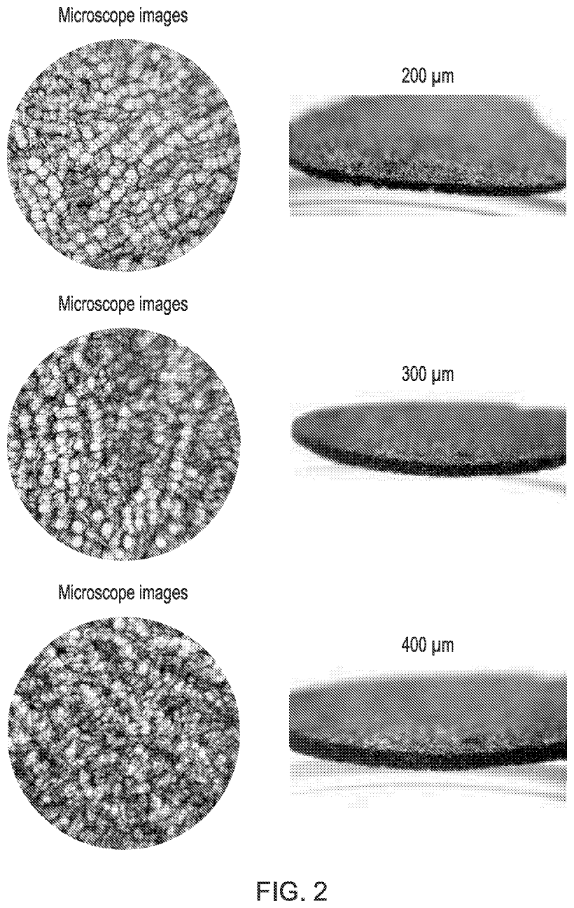

The porous scaffold membranes described herein may have a thickness from 100 μm to about 1000 μm. In some examples the porous membranes may have a thickness from about 200 μm to about 600 μm. In some examples the porous membranes may have a thickness from about 300 μm to about 500 μm. In some examples the porous membranes may have a thickness of about 400 μm. Examples of membranes with differing thickness are shown in FIG. 2.

The porous scaffold membranes provided herein are conducting scaffolds. That is to say that the porous scaffold membranes described herein are capable of conducting electricity. The conducting properties of the porous scaffold membranes may be provided by including conducting polymers in the porous scaffold membrane.

Conducting polymers include any polymers that have ionic and electronic conductivity properties. Conducting polymers also include composites with the ability to conduct electronic charge or ions, and hybrid polymer-metal materials that are electrically or ionically conductive. Polymers that may be used include one or more semiconducting polymers such as: polythiophenes (PTh); poly(pyrroles); polyanilines; polyacetylenes polythiophene polymer; and blends thereof. Some semi-conducting polymers may include dopants in order to provide conducting polymers, in other cases the polymers may be self-doped polymers that do not require dopants to provide conducting polymers.

In particular, the porous scaffold membranes provided herein may include a semi-conducting polymer comprising poly(3,4-ethylenedioxythiophene) (PEDOT). The conducting polymer may include a dopant such as poly(3,4-ethylenedioxythiophene) polystyrene sulfonate (PSS). In some examples, the conducting polymer may comprise poly(3,4-ethylenedioxythiophene) polystyrene sulfonate (PEDOT:PSS). The dopant may be any substance that provides a semi-conducting polymer with conductor properties. In some examples, the dopant may be dextran sulfate or tosylate. For example, the conducting polymer may be PEDOT:Tos or PEDOT:dextran sulphate. Preferably, the dopant and/or polymer has a relatively high amount of water to allow for freeze drying of the conducting polymer.

The porous scaffold membranes provided herein may have a conductivity of from 5 to 100 S/cm. For example, 5, 10, 15, 20, 30, 40, 50, 60, 70, 80, 90, 100 S/cm. In some examples, the porous scaffold membranes may have a conductivity from 5 to 50 S/cm. In some examples, the porous scaffold membranes may have a conductivity from 5 to 20 S/cm. In some examples, the porous scaffold membranes may have a conductivity from 5 to 15 S/cm. In some examples, the porous scaffold membranes may have a conductivity from 8 to 13 S/cm. The conductivity of the porous scaffold membrane may be altered, for example increased or decreased by the inclusion of additives or additional agents.

Conductivity may be measured using any known methods such as two-probe resistance measurement, methods using a 4-point probe system, methods using a multimeter, or impedance spectroscopy.

The porous scaffold membranes provided herein may have an impedance of between 1 and 500 Ohm. In some examples, the porous scaffold membranes provided herein may have an impedance of between 10 and 200 Ohm. In some examples, the porous scaffold membranes provided herein may have an impedance of between 20 and 10 Ohm. In some examples, the porous scaffold membranes provided herein may have an impedance of between 50 and 80 Ohm. As impedance may be dependent on the method used to measure the impendence and/or the thickness of the porous scaffold membrane, the impendence may be measured in the presence of phosphate buffered saline at 1 kHZ and for a porous scaffold membrane having a thickness of 500 μm. For example, the impendence may be between 50 and 80 Ohms in the presence of phosphate buffered saline at 1 kHZ and a thickness of 500 μm. In some examples, the porous scaffold membranes may have an impendence in the presence of phosphate buffered saline of between 58 and 74 Ohm at 1 kHZ and a membrane thickness of 500 μm.

The porous scaffold membranes provided herein may include one or more additional agents. For example, the porous scaffold membranes may include one or more of: at least one cryoprotectant; at least one oligosaccharide; at least one polymer; at least one conductivity enhancing agent; at least one biopolymer; and/or at least one crosslinking agent.

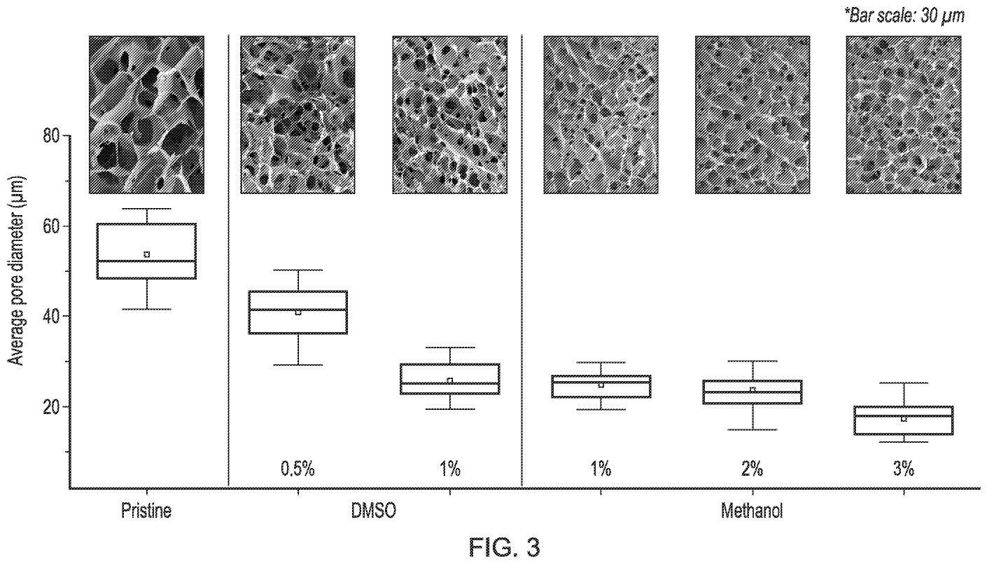

Cryoprotectants refers to agents that inhibit or prevent freezing. Cryoprotectants may control water crystallization in the polymer solution during freeze drying used to form the porous scaffold membranes as well modify pore morphology. Pore morphology is used herein to refer to the shape, size, and/or spatial arrangement of pores of the porous scaffold membranes described herein. For example, cryoprotectants may decrease the average pore size and pore structure as seen in FIG. 3.

The amount of cryoprotectant that may be included may be from 0.1% to about 10% by weight (wt). For example, one or more cryoprotectants may be included at an amount of 0.1, 0.2, 0.3, 0.4, 0.5, 0.6, 0.7, 0.8, 0.9, 1, 2, 3, 4, 5, 6, 7, 8, 9, or 10 wt %. In some examples the cryoprotectant may be included at an amount of from 0.5% to about 5 wt %. In some examples the cryoprotectant may be included at an amount of from 0.5% to about 3 wt %. In some examples, the amount of cryoprotectant that may be included may be at least 0.1 wt %.

Example cryoprotectants include small polyols (such as glycerol and ethylene glycol), alcohols (such as methanol), and sugars (such as sucrose). Cryoprotectants may also act as conductivity enhancing agents in some cases.

In certain examples the cryoprotectant may be dimethyl sulfoxide (DMSO), and/or methanol. In certain examples the cryoprotectant is dimethyl sulfoxide (DMSO). In certain examples the cryoprotectant is methanol.

In some examples, the porous scaffold membrane may include 0.5 wt % DMSO. In some examples, the porous scaffold membrane may include 1 wt % methanol. In some examples, the porous scaffold membrane may include 2 wt % methanol. In some examples, the porous scaffold membrane may include 3 wt % methanol.

Cryoprotectants may be used as additives when carrying out methods of forming the porous scaffold membranes provided herein and as such may not be included in the final porous scaffold membranes. However, small of amounts of cryoprotectants may be incorporated into the porous scaffold membranes. In some examples, cryoprotectants may be incorporated into the porous scaffold membranes in the same amount as used in methods of making the porous scaffold membrane.

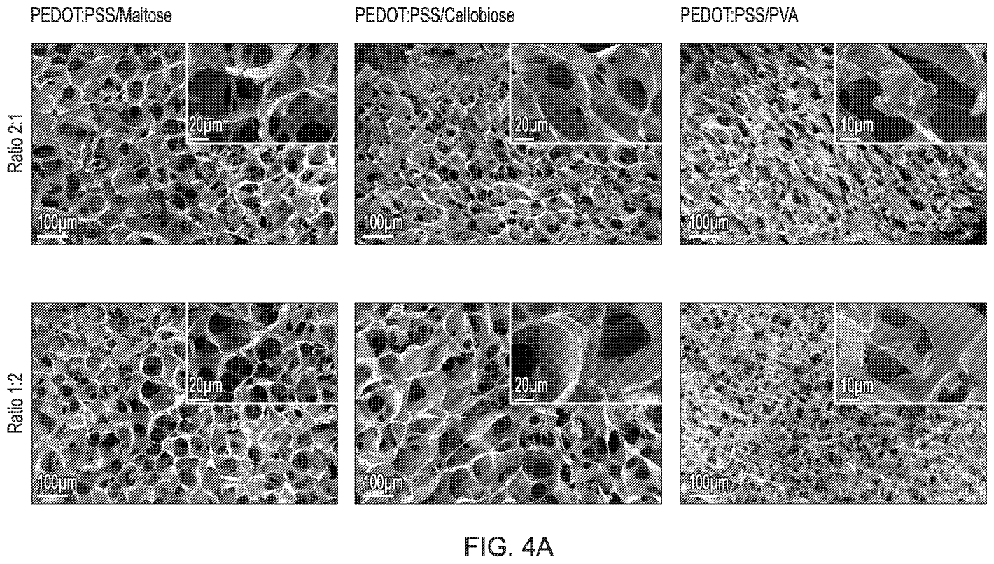

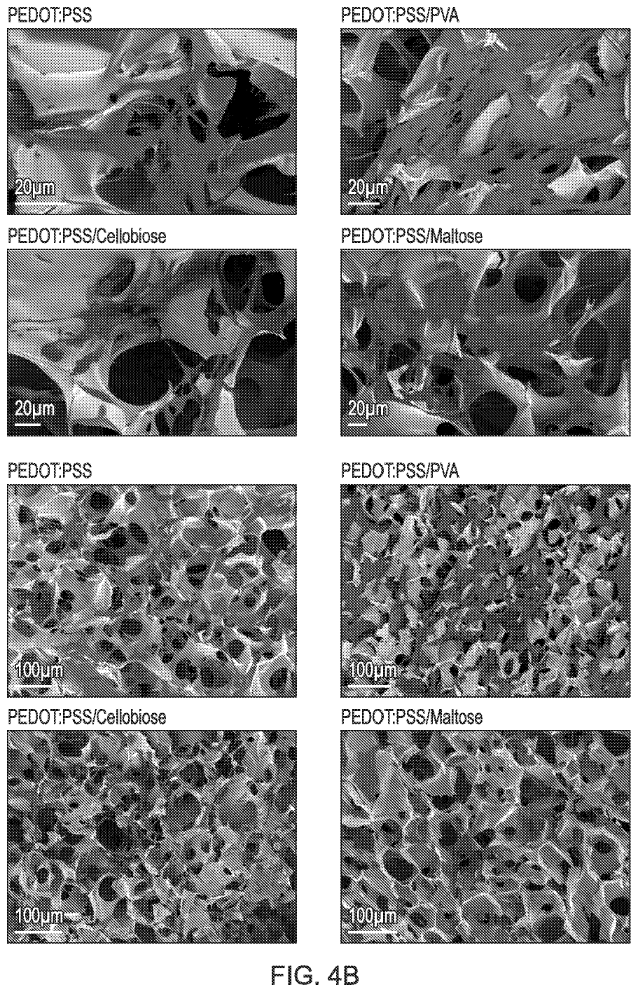

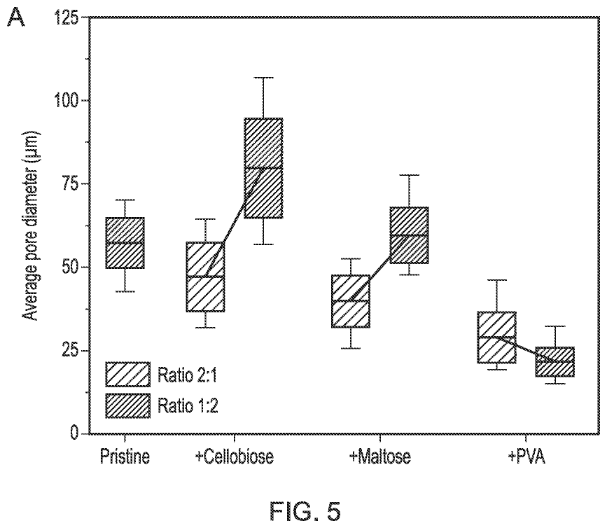

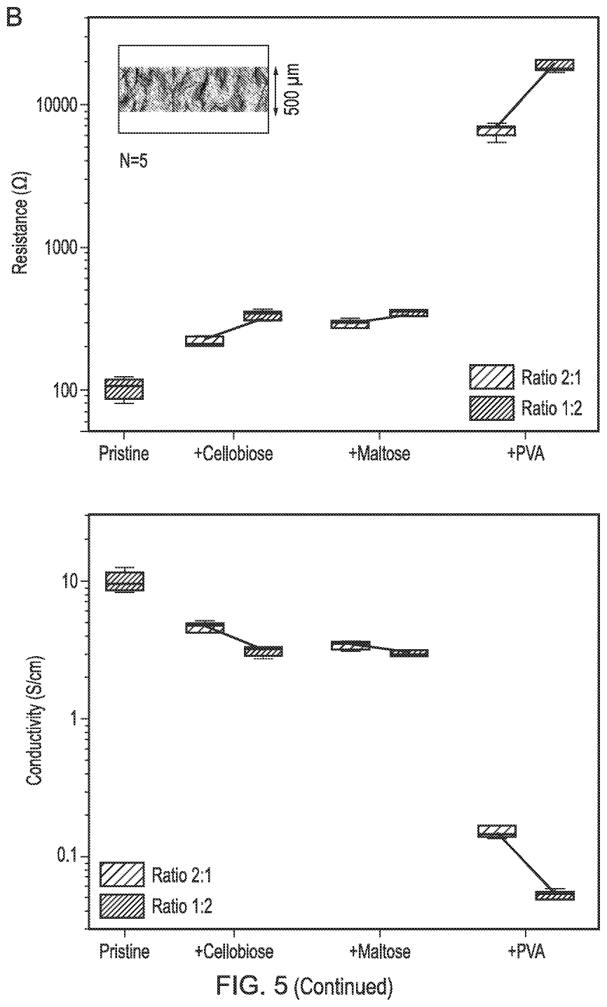

The inclusion of oligosaccharides, biopolymers and/or polymers in the porous scaffold membranes may alter pore morphology, electrical properties (such as conductivity, impendence and/or resistance) and/or physical properties of the porous scaffold membranes. Physical properties include stiffness, conductivity, brittleness, swelling (water uptake), and/or topography. Examples of porous scaffold membranes including oligosaccharides, biopolymer or polymers are shown in FIGS. 4 and 5.

Oligosaccharide, refers to any carbohydrate of from two to six units of simple sugars (monosaccharides). Examples of oligosaccharides that may be included in the porous scaffold membranes include maltose, cellobiose, starch, glycogen, cellulose, chitin, galactogen, sucrose, lactose, and/or trehalose.

Additional polymers that may be included in the porous scaffold membranes provided herein may be any water soluble polymer. Examples of polymers that may be included in the porous scaffold membranes include polyvinyl(alcohols), polyacrylates, water-soluble acrylate copolymers, polyvinylpyrrolidone, polyethyleneimine, purulan, water-soluble natural polymers, polyalkylene oxides, polyacrylamides, polyacrylic acids and salts thereof, cellulose, cellulose ethers, cellulose esters, cellulose amides, vinyl acetate, polycarboxylic acids and salts thereof, polyamino acids and polyamides.

Biopolymers include, but are not limited to, proteins and/or polysaccharides. Biopolymers that may be included may be extracellular matrix proteins such as fibronectin and laminin, and/or extracellular matrix polysaccharides such as hyaluronic acid. Other examples of biopolymers that that may be included in the porous scaffold membranes include collagen, glycogen, cellulose, chitin, galactogen, poly-L/D lysine, starch (including amylose and/or amylopectin), chitosan, hemicellulose, lignin, alginate, dextran, pulllanes, polyhydroxyalkanoate, fibrin, cyclodextrins, proteins (e.g. soy proteins), polysaccharides (e.g. pectin), and/or polylactic acid. The biopolymer may be a proteoglycan such as glycosaminoglycan-containing molecules, chondroitin sulfate, dermatan sulphate, heparan sulphate, keratan sulphate, hyaluronan, fibrin, elastin, fibronectin, laminin, glycosaminoglycan, nidogen and/or collagen.

Oligosaccharides, biopolymers and/or polymers may be included at a volume to volume (v/v) ratio of 2:1 of oligosaccharide, biopolymer and/or polymer to the conducting polymer. For example, the porous scaffold membranes may include at least one oligosaccharide at a v/v ration of 2:1 of oligosaccharide to conducting polymer (for example PEDOT:PSS). For example, the porous scaffold membranes may include at least one polymer at a v/v ration of 2:1 of polymer to conducting polymer (for example PEDOT:PSS). For example, the porous scaffold membranes may include at least one biopolymer at a v/v ration of 2:1 of biopolymer to conducting polymer (for example PEDOT:PSS).

Oligosaccharides, biopolymers and/or polymers may be included at a volume to volume (v/v) ratio of 1:2 of oligosaccharide, biopolymer and/or polymer to the conducting polymer. For example, the porous scaffold membranes may include at least one oligosaccharide at a v/v ration of 1:2 of oligosaccharide to conducting polymer (for example PEDOT:PSS). For example, the porous scaffold membranes may include at least one polymer at a v/v ration of 1:2 of polymer to conducting polymer (for example PEDOT:PSS). For example, the porous scaffold membranes may include at least one biopolymer at a v/v ration of 1:2 of biopolymer to conducting polymer (for example PEDOT:PSS).

Oligosaccharides, biopolymers and/or polymers may be added to a solution comprising the conducting polymer during methods of making a porous scaffold membrane as described herein. Oligosaccharides, biopolymers and/or polymers may be in solution before being added to a solution comprising the conducting polymer.

For example, oligosaccharides, biopolymers and/or polymers may be diluted to at least 1% in water. For example, 1%, 2%, 3% or 4% or more in water.

The final concentration of oligosaccharides, biopolymers and/or polymers in a porous scaffold membrane may be at least 1%. For example at least 1%, 2%, 3%, 4% or more.

Crosslinking agents may be included in the porous scaffold membranes provided herein in order to improve physical properties of the porous scaffold membranes. For example stiffness, brittleness, swelling and/or conductivity. Crosslinkers may act as an adhesive that helps prevent the porous scaffold membranes from breaking. In some instances, crosslinkers may also provide sites for functionalisation of the porous scaffold membranes. For example, crosslinkers may be used for the attachment of proteins or other molecules to the porous scaffold membranes.

Examples of crosslinking agents that may be included in the porous scaffold membranes provided herein include: 3-glycidyloxypropyl)trimethoxysilane (GOPS); glutaraldehyde, acylating compounds, adipyl chloride, aldehydes, alkyl and aryl halides, bisimidates, carbodiimides, divinyl sulfone (DVS), formaldehyde, glyoxal, hexamethylene diisocyanate, hydroxychloride, hydroxyethyl methacrylate divinyl sulfone (HEMA-DIS-HEMA), imidoesters, isocyanates, N-hydroxysuccinimide, N-substituted maleimides, polyaldehyde, diphenylphosphoryl azide (DPPA), polyepoxy compounds comprising backbone of 17-25 carbons and 4-5 epoxy groups, polyepoxy ethers, polyethylene glycol divinyl sulfone (VS-PEG-VS), polyglycerol polyglycidyl ether and combinations thereof.

Crosslinking agents may be added at an amount of at least 0.1 wt %. For example, at least 0.1, 0.2, 0.3, 0.4, 0.5, 0.6, 0.7, 0.8, 0.9, 1, 2, 3, 4, 5, 6, 7, 8, 9, or 10 wt %

Conductivity enhancing agents refers to any agent that may alter the electrical properties of the porous scaffold membranes described herein. For example, conductivity enhancing agents may increase conductivity of the porous scaffold membranes described herein.

Conductivity enhancing agents include, carbon nanotubes, dodecylbenzenesulfonic acid (DBSA), and/or graphene. As detailed herein, other additional agents described herein may also have effects on the conductivity of the porous scaffold membranes.

Carbon nanotubes that may be included may be single walled carbon nanotubes (SWCNT) and/or multi-walled carbon nanotubes (MWCNT).

Conductivity enhancing agents may be added at an amount of at least 1 wt %. For example, at least 0.1, 0.2, 0.3, 0.4, 0.5, 0.6, 0.7, 0.8, 0.9, 1, 2, 3, 4, 5, 6, 7, 8, 9, or 10 wt %.

The porous scaffold membranes provided herein may be produced by a method comprising:
a. forming an aqueous dispersion of a semi-conducting polymer and a dopant;
b. adding a crosslinking agent to the aqueous dispersion; and
c. lyophilising the aqueous solution.

An aqueous dispersion is a liquid system in which solid particles are uniformly dispersed in water. The solid particles in the case of the porous scaffold membranes described herein are particles of the semi-conducting polymer and dopant. For example, an aqueous dispersion of poly(3,4-ethylenedioxythiophene) and polystyrene sulfonate.

Methods of making an aqueous dispersion are well known. Alternatively, aqueous dispersions of, for example, poly(3,4-ethylenedioxythiophene) and polystyrene sulfonate may be commercially available, for example CLEVIOS™ PH 1000 available from Heraeus.

The concentration of semi-conducting polymer and/or conducting polymer in aqueous dispersion may be at a concentration of 1.25 wt %.

A crosslinking agent is added to the aqueous dispersion. The crosslinking agent may be a crosslinking agent as described herein. For example, GOPS and/or glutaraldehyde may be added to the aqueous dispersion. In some examples, GOPS may be added to the aqueous dispersion. In some examples, glutaraldehyde may be added to the aqueous dispersion.

The crosslinking agent may be added in an amount of at least 0.1 wt %. For example, at least 0.1, 0.2, 0.3, 0.4, 0.5, 0.6, 0.7, 0.8, 0.9, 1, 2, 3, 4, 5, 6, 7, 8, 9, or 10 wt %

For example, 3% GOPS may be added to the aqueous dispersion.

At least one cryoprotectant; at least one oligosaccharide; at least one polymer; at least one conductivity enhancing agent; at least one biopolymer; and/or at least one further crosslinking agent as described herein may also be added to the aqueous dispersion.

For example, a conductivity enhancing agents may be added at an amount of at least 0.1 wt %. For example, at least 0.1, 0.2, 0.3, 0.4, 0.5, 0.6, 0.7, 0.8, 0.9, 1, 2, 3, 4, 5, 6, 7, 8, 9, or 10 wt %. In some examples, the conductivity enhancing agent is DBSA. In some examples, the conductivity enhancing agent is SWCNT. In some examples, 0.5 wt % of DBSA is added to the aqueous dispersion. In some examples, SWCNT are added at a concentration of 0.5 wt % to the aqueous dispersion.

For example, an oligosaccharide is added to the aqueous dispersion. The oligosaccharide may be added at a v/v ratio of 2:1 to conducting polymer (e.g. PEDOT:PSS). The oligosaccharide may be added at a v/v ratio of 1:2 to conducting polymer (e.g. PEDOT:PSS).

The oligosaccharide may be diluted in water prior to addition to the aqueous dispersion. For example, the oligosaccharide may be diluted to a concentration of at least 1% prior to addition to the aqueous dispersion. In some examples, maltose diluted to a concentration of 2% is added in a v/v ratio of 1:2 to conducting polymer (e.g. PEDOT:PSS). In some examples, maltose diluted to a concentration of 2% is added in a v/v ratio of 2:1 to conducting polymer (e.g. PEDOT:PSS). In some examples, cellobiose diluted to a concentration of 4% is added in a v/v ratio of 1:2 to conducting polymer (e.g. PEDOT:PSS). In some examples, cellobiose diluted to a concentration of 4% is added in a v/v ratio of 2:1 to conducting polymer (e.g. PEDOT:PSS).

For example, a polymer is added to the aqueous dispersion. The polymer may be added at a v/v ratio of 2:1 to conducting polymer (e.g. PEDOT:PSS). The polymer may be added at a v/v ratio of 1:2 to conducting polymer (e.g. PEDOT:PSS).

The polymer may be diluted in water prior to addition to the aqueous dispersion. For example, the oligosaccharide may be diluted to a concentration of at least 1% prior to addition to the aqueous dispersion. In some examples, polyvinyl alcohol diluted to a concentration of 4% is added in a v/v ratio of 1:2 to conducting polymer (e.g. PEDOT:PSS). In some examples, polyvinyl alcohol diluted to a concentration of 4% is added in a v/v ratio of 2:1 to conducting polymer (e.g. PEDOT:PSS).

For example, a biopolymer is added to the aqueous dispersion. The biopolymer may be added at a centration of at least 0.01 wt % to the aqueous dispersion.

A biopolymer may be added at a concentration of 0.05 wt %. For example, 0.05 wt % of collagen may be added to the aqueous dispersion.

Cryoprotectants may be added to the aqueous dispersion. The amount of cryoprotectant that may be include may be from 0.1% to about 10% by weight (wt). For example, one or more cryoprotectants may be included at an amount of 0.1, 0.2, 0.3, 0.4, 0.5, 0.6, 0.7, 0.8, 0.9, 1, 2, 3, 4, 5, 6, 7, 8, 9, or 10 wt %. In some examples the cryoprotectant may be included at an amount of from 0.5% to about 5 wt %. In some examples the cryoprotectant may be included at an amount of from 0.5% to about 3 wt %.

For example, methanol may be added to the aqueous dispersion at a concentration of 1 wt %. For example, methanol may be added to the aqueous dispersion at a concentration of 2 wt %. For example, methanol may be added to the aqueous dispersion at a concentration of 3 wt %.

For example, DMSO may be added to the aqueous dispersion at a concentration of 0.5 wt %. For example, DMSO may be added to the aqueous dispersion at a concentration of 1 wt %.

The addition of different agents to the aqueous dispersion and the amounts thereof may have different effects of the properties of the porous scaffold membrane produced. The agents added and amounts added may be selected based on the desired final use of the porous scaffold membrane. For example, the types of cells for which the porous membrane scaffold is to be used for culturing.

After the aqueous dispersion has been formed and any additional agents added, the aqueous dispersion is lyophilised. Lyophilisation, also known as freeze drying or cryodesiccation removes water from the aqueous dispersion, thus leaving a porous scaffold comprising the conducting polymer and any optional additional agents.

The aqueous dispersion may be poured into a mould or cast prior to lyophilisation in order to provide a porous scaffold having a specific shape. For example, the aqueous dispersion may be poured into a well of a well plate or a part of a well plate insert in order to provide a porous scaffold that has a shape that conforms to the well or part of the insert.

In order to provide a porous scaffold membrane, the porous scaffold membrane may be sliced to a desired thickness. Methods of slicing or cutting porous scaffolds are well known, such as the use of a vibratome.

The porous scaffold membranes described herein may be incorporated into a device for dynamically monitoring a 3-dimensional cell culture. Such devices or apparatus include an insert for use with a well plate, such as a multi-well plate. As such, provided herein are inserts for dynamically monitoring 3-dimensional cell cultures.

The use of the porous scaffold membranes as described herein in an insert allows for the study of various 3-dimensional cell systems in a high throughput manner. Integration of porous scaffold membranes as described herein into inserts that may be used with multi-well plates may allow for simultaneous study of multiple 3-dimensional cell systems.

Inserts include a porous scaffold membrane as described herein in electrical contact with at least one working electrode. The insert also includes a body that supports the porous scaffold membrane and can be inserted into a support device, such as a well plate, so as to suspend the porous scaffold membrane within support device, for example support the porous scaffold membrane within a well of a well plate so that the porous scaffold membrane is not in contact with a bottom surface of the well of the well plate.

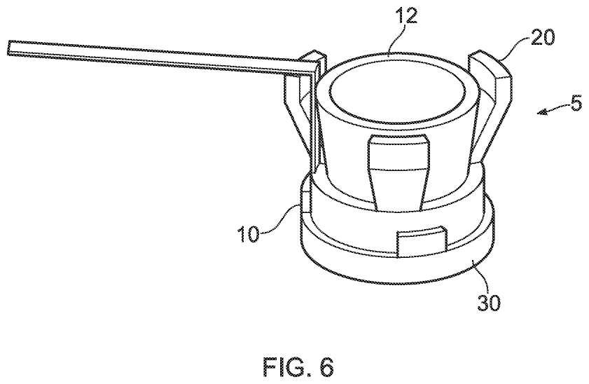

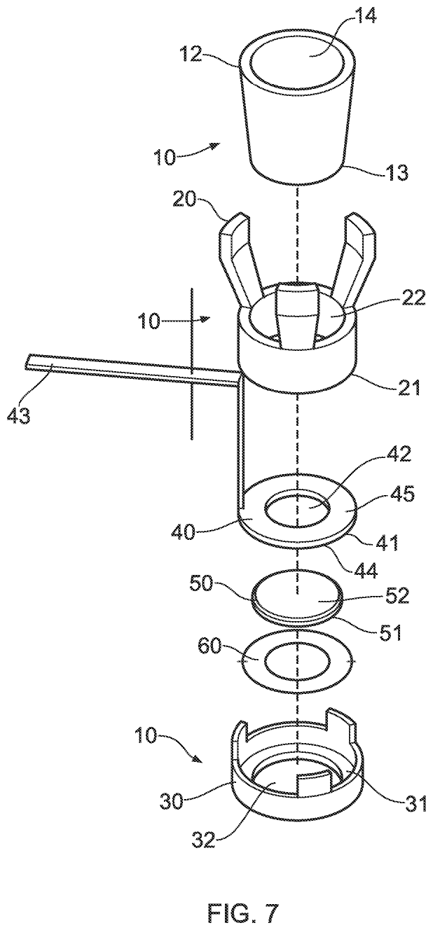

FIG. 6 shows an insert (5). The insert having a body (10) which includes a well portion (12), an upper portion (20) and a lower portion (30). FIG. 7 shows an insert with a first example arrangement.

As shown in FIG. 7, the well portion (12) is a hollow tube structure with an aperture (14) extending therethrough. In the example shown, the well portion (12) is a hollow cylinder (e.g. is a tube with a circular cross section). It will be appreciated that the well portion may be a tube having any cross-sectional shape, such as square, rectangular, or polygonal. The shape of the well portion (12) may be selected based on the shape of the upper (20) and lower (30) portions of the body as well as based on the shape of the well the insert is to be placed into.

The upper portion (20) includes an annular structure with an aperture (22) which is configured to receive the well portion (12).

The well portion (12) is positioned within the upper portion (20) so that the lower surface (13) of the well portion is flush with a lower surface (21) of the upper portion. The well portion (12) and upper portion (20) may be held together by friction or by use of an adhesive, such as PDMS.

In the example shown in FIG. 7, a working electrode (40) is positioned at the lower surface (21) of the upper portion and lower surface (13) of the well portion. The working electrode (40) includes a ring portion (41) having an aperture (42). The working electrode (40) also includes a connector (43) which extends away from the ring portion (41) to a point above an upper surface of the well portion (12). The connector (43) is configured so that when the insert is positioned in a well, the connector extends out of the well. The connecter may then be connected to one or more electrical devices outside of the well. The electrode may be referred to as “an O-ring electrode” given its annular shape and aperture.

The working electrode may include any material capable of conducting electricity. For example, the working electrode may include a metal. Suitable metals include gold, platinum, palladium, Indium-tin-oxide and/or graphite. In certain examples, the working electrode is made from gold. The working electrode may also include a flexible polymer. For example, the flexible polymer may be Kapton, polyethylene naphthalate (PEN), Parylene C or any suitable flexible polymer. The conductive material may be deposited onto the external surfaces of the flexible polymer in order to provide a flexible working electrode. Gold may have the advantages of high conductivity, less contact resistance, corrosion resistance, and/or biocompatibility.

A porous scaffold membrane (50) as described herein is positioned in contact with the working electrode (40). In the example shown in FIG. 7, the porous scaffold membrane (50) is located in contact with a lower surface (44) of the ring portion (i.e. the ring portion (41) is located on an upper surface (51) of the porous scaffold membrane). In other examples, the porous scaffold membrane (50) may be located in contact with an upper surface (45) of the ring portion (41) of the working electrode (40) (i.e. the ring portion (41) is located on a lower surface (52) of the porous scaffold membrane). That is to say that the working electrode (40) may be located above and/or below the porous scaffold membrane. The aperture (42) of the working electrode may be aligned with the aperture (14) of the well portion (12) in order to provide at least a portion of an exposed surface of the porous scaffold membrane (50) when in use.

The working electrode (40) may also include an insulating layer deposited on one or more surfaces of the ring portion (41) and/or the connector (42). The insulating may be deposited onto one or more surface of the working electrode which are in contact with liquid when in use (i.e. not in contact with the porous scaffold membrane). For example, when the upper surface (45) of the ring portion is in contact with the porous scaffold membrane (50) the lower surface (44) of the ring portion may include an insulating layer. In the case wherein the lower surface (44) of the ring portion is in contact with the porous scaffold membrane (50) the upper surface (45) of the ring portion may include an insulating layer.

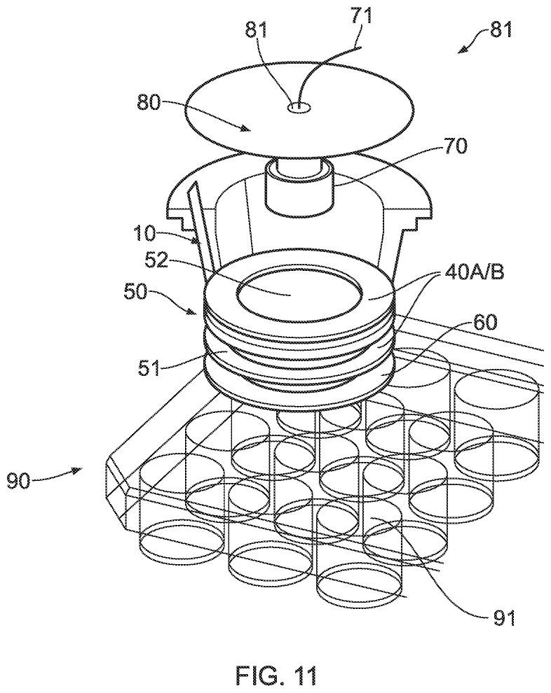

In some examples, (as shown in FIG. 11) the insert may include two working electrodes, a first working electrode (40A) positioned with the lower surface of the ring portion (41) in contact with an upper surface (51) of the porous scaffold membrane and a second working electrode (40B) with the upper surface of a ring portion in contact with the lower surface (52) of the porous scaffold membrane. As such, the upper surface of the ring portion of the first working electrode may include an insulating layer and the lower surface of the ring portion of the second working electrode may include an insulating layer.

An insulating layer may also be deposited on the surfaces of the connector of each working electrode.

The insulating layer may help to prevent electrical interference from liquid in contact with the working electrode when the insert is in use. The insulating layer may be any suitable material that does not conduct electricity. For example, the insulating layer may be formed from PDMS or Parylene-C.

With reference to FIG. 7, the lower portion (30) connects to the upper portion (30) in order to house the porous scaffold membrane (50) and the ring portion (41) of the working electrode (40). The lower portion (30) may include a flange (31) positioned so as to engage with the porous scaffold membrane or the ring portion (41) of the working electrode and hold the ring portion (41) of the working electrode and/or porous scaffold membrane in place. The lower portion includes an aperture (32). When in use the aperture (32) of the lower portion is configured so as to provide an exposed surface of the porous scaffold membrane. The aperture (32) of the lower portion may be aligned with the aperture (42) of the ring portion (41) of the working electrode (40) when the porous scaffold membrane (50) is located in contact with the upper surface (45) of the working electrode (40).

The lower portion (30) may be connected to the upper portion (12) by any suitable connecting means. For example, by friction (e.g. push fit), by adhesive (such as PDMS), or by threaded engagement to a corresponding threaded portion of the upper portion (12).

Optionally, the insert may also include a membrane support member (60) located between the flange (31) of the lower portion (30) and the porous scaffold membrane (50) as in the arrangement shown in FIG. 7 or between the flange (31) of the lower portion and the working electrode, when at least one working electrode is positioned in contact with a lower surface of the porous scaffold membrane. The membrane support member (60) includes an aperture configured to provide at least a portion of an exposed surface of the porous scaffold membrane when in use. The aperture of the support member may be coaxially aligned with the aperture (32) of the lower portion (30), the aperture (42) of the one or more working electrodes and/or the aperture (14) of the well portion. Thus providing a structure that has at least a portion of the upper and lower surface of the porous scaffold membrane exposed to liquid within a well or air when in use.

The membrane support member (60) may be made from any suitable material that does not conduct electricity, such as non-conducting plastic.

The insert is configured so that in use, the porous scaffold membrane is in electrical contact with the working electrode and may be disposed in a support device, such as in a well of a well plate so that the porous scaffold membrane is suspended within the support device (e.g. suspended within a well). That is to say that the porous scaffold membrane is maintained in a position wherein it is not in contact with a lower surface of the support device, such as the bottom of a well plate.

The body described above and shown in FIG. 7 is an example of a body configured to deposited into a support device. It will be understood that the body may be any suitable device that can be disposed into a support device, such as a well of well plate and maintain the porous scaffold membrane in a suspended position within the support device, such as suspended in a well of a well plate. Suspending a porous scaffold membrane ensures that at least a lower surface (51) of the porous scaffold membrane is in contact with liquid inside a support device, such as culture medium within a well of a well plate. The porous scaffold membrane may be suspended in any suitable way. For example, the insert may include a flange on the upper portion that abuts the top surface of a well plate so that the lower portion of the insert, and therefore the porous scaffold membrane, are positioned within the well above the lower surface of the well. In another example, the insert may include well engaging elements that extended outwards from the upper and/or lower portion of the insert to engage with the walls of a well. The well engagement elements contact the walls of the well when the insert is disposed therein. The engagement elements cause friction with the walls of the well thus preventing the insert from dropping into the bottom of the well. In another example, the insert is disposed in a well so that the lower surface of the lower portion abuts the lower surface of the well. The material between the lower surface of the lower portion and the flange prevent the porous scaffold membrane from contacting the lower surface of the well.

Examples of bodies that may be used to provide an insert as described herein include the CellCrown™ available from Scaffdex.

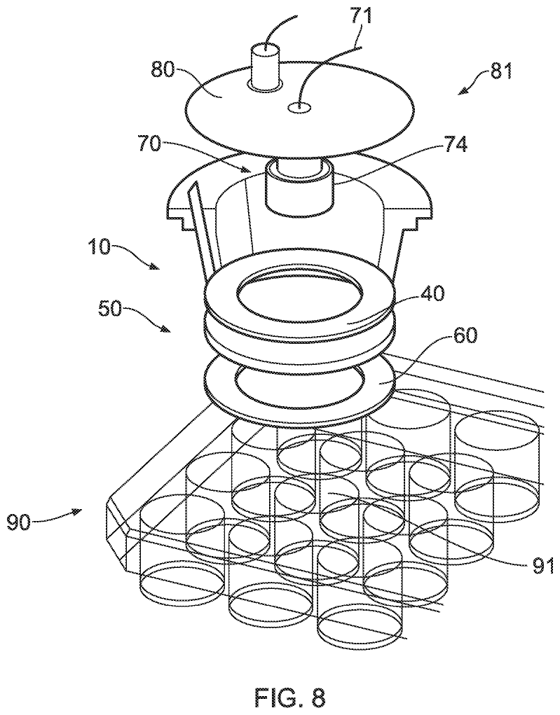

FIG. 8 shows one example of an insert having a single working electrode. The insert shown includes a counter electrode (70). The counter electrode (70) is positioned in an apical (upper) portion of the insert. The counter electrode (70) may be made of any material capable of conducting electricity. For example, the counter electrode may be made from a metal. In particular, the counter electrode may be made from a mesh. For example, the counter electrode may comprise a platinum, stainless steel or rhodium mesh. An alternative counter electrode may be a reticulated glassy (vitreous) carbon porous electrode. The use of a mesh material may help improve the surface area of the counter electrode and therefore help improve electrical measurements. In some examples, the counter electrode may comprise a porous scaffold membrane as described. In such examples, the porous scaffold membrane acting as a counter electrode would not be seeded with cells and is isolated from any cells to prevent cells infiltrating the scaffold or attaching to the surface of the scaffold.

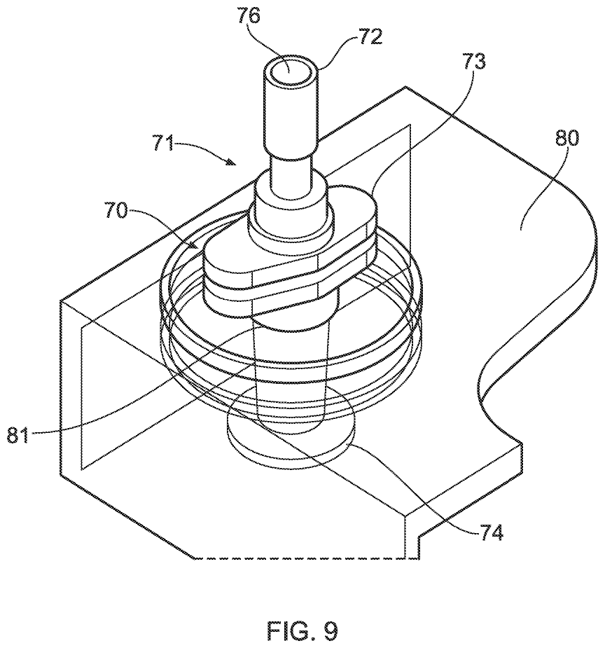

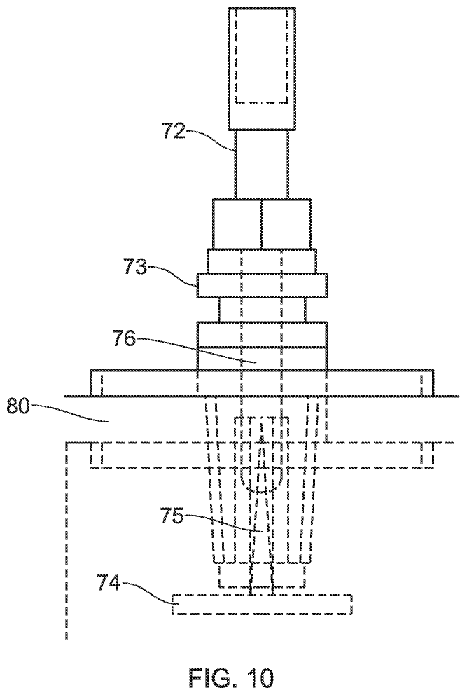

The counter electrode shown in FIG. 8 is integrated with a lid (80) that may be a lid of the insert or support device that covers the apical portion of the insert. The lid (80) includes an opening (81) to allow connection of the counter electrode to other electronic devices via a connector (71). FIGS. 9 and 10 show an example of a lid with an integrated counter electrode.

The counter electrode includes a connector (71). The connector may be any suitable connecter adapted to connect the working electrode to other external electrical devices, such as cables or wires. Shown in FIGS. 9 and 10, the connector includes a holder (73) that is positioned within the opening (81) in the lid. The holder (73) may be any suitable holding device that can maintain the counter electrode in place. For example, the holder may be a luer cap and plug device. The connector shown in FIGS. 9 and 10 includes a metal pin (72) which is inserted into the holder (73) via an opening extending through the centre of the holder (73). The metal pin may be any material that is capable of conducting electricity such as gold. The metal pin extends through the holder and through the opening (81).

The counter electrode includes an electrode (74) that has a protrusion (75) extending from a first surface. The protrusion (75) extends from the first surface of the electrode into an opening of the holder so that the electrode is in electrical contact with the pin.

The electrode (74) is secured in place by a connection with the pin and/or the holder. The connection may be made by a fastening device, such as a crimp or by an adhesive.

The electrode (74) is secured so that, in use, the electrode (74) is positioned within an upper (apical) portion of an insert. The electrode (74) is positioned so that in use it may be submerged in liquid, such as cell culture medium, in order to provide an electrical connection between the electrode (74), the porous scaffold membrane (50) and the working electrode (40). Other suitable arrangements for positioning the counter electrode in an upper portion of the insert or in an upper portion of the support device, such as an upper portion of a well, will be known.

With reference to FIG. 8, the lid (80) and counter electrode (70) may be positioned over the top of an insert (10) when the insert is disposed within a support device (90). In certain examples, the lid may be a lid for a support device. For example, the lid may be a lid of a support device that includes a number of wells (91), such as a multi-well plate with an insert in each well. As such, the lid may include a plurality of integrated counter electrodes each corresponding to each well, so that each well has a counter electrode positioned within an apical portion of the insert when the insert is disposed within a well and the lid is positioned over the support device so as to cover the insert and/or well.

As such, the lid and counter electrode integrated therein may be considered to be part of the support device in some examples.

The example insert shown in FIGS. 7 to 10 may allow for the insert be operated as an electrode as described herein.

In another example, the insert may include two or more working electrodes. With reference to FIG. 11, the insert includes a first working electrode (40A) and a second working electrode (40B) with ring portions positioned on opposing sides of the porous scaffold membrane. As shown in FIG. 11, the ring portion of the first working electrode (40A) is located in contact with an upper surface (52) of the porous scaffold membrane and the ring portion of the second working electrode is in contact with a lower surface (51) of the porous scaffold membrane. Both the first and second working electrodes may be working electrodes as described herein.

As such, both the first and second working electrodes may include a ring portion having an aperture. The first and second working electrodes may also include a connector each which extends away from each respective ring portion to a point above an upper surface of the well portion of the insert. The connectors are configured so that when the insert is positioned in a well, the connectors extend out of the well. The connectors may then be connected to one or more electrical devices outside of the well.

The insert arrangement shown in FIG. 11 may allow the insert to be operated as a transistor or diode as described herein.

In both examples shown in FIGS. 7 to 10 and FIG. 11, the insert may be disposed into a support device, such as a well of a well plate. A liquid such as cell culture medium may then be added to the well in order to submerge both the upper and lower surface of the porous scaffold membrane. A lid including a counter electrode may then be positioned over the well so that the counter electrode is submerged in the liquid in an upper (apical) portion of the well. The liquid act as an electrolyte and provides an electrical contact between the porous scaffold membrane, at least one working electrode and the counter electrode thereby allowing electrical interrogation of the porous scaffold membrane and any cells cultured thereon and/or therein.

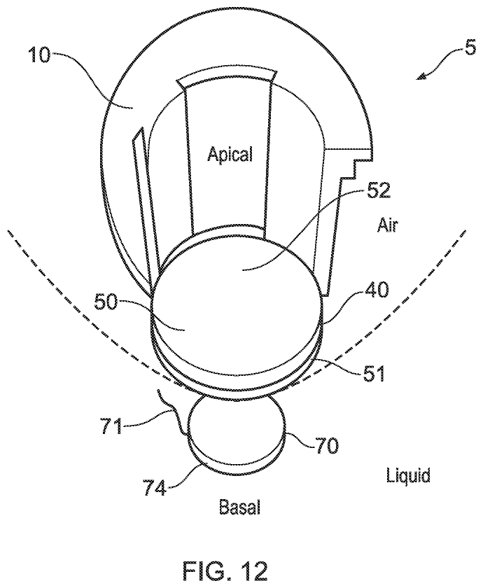

In another example, shown in FIG. 12, the counter electrode may be positioned in a lower (basal) position with respect to the insert. For example, in a lower (basal) portion of a support device. With reference to FIG. 12, the electrode (74) of the counter electrode (70) is located below the insert (5). That is to say that the electrode (74) of the counter electrode is located between a lower surface of a support device and a lower surface of the insert (5). The counter electrode is positioned so that in use, the electrode (74) is submerged in liquid. The connector (71) connects the electrode (74) of the counter electrode (70) to one or more external electrical devices. The connector may be a wire or cable that extends from the electrode (74) out of the support device. The connector is configured so that it does not form electrical contact with the working electrode or porous scaffold membrane in order to prevent a short circuit.

As shown in FIG. 12 an upper surface (52) of the porous scaffold membrane (50) is positioned so as to be exposed to the air when in use. The working electrode is located in contact with a lower surface (51) of the porous scaffold membrane (5). The lower surface (51) of the porous scaffold membrane and the working electrode are positioned so that in use the lower surface (51) of the porous scaffold membrane and the working electrode are submerged in liquid and thus in electrical contact via an electrolyte (such as cell culture medium) when in use.

The arrangement shown in FIG. 12 may provide a device that can be used to interrogate and study cells at an air-liquid-interface. For example, the device shown in FIG. 12 may be used to provide a model respiratory organoid or system. Cells on the upper surface (52) of the scaffold are exposed to the air while cells within the scaffold and on the lower surface (51) are submerged in culture medium.

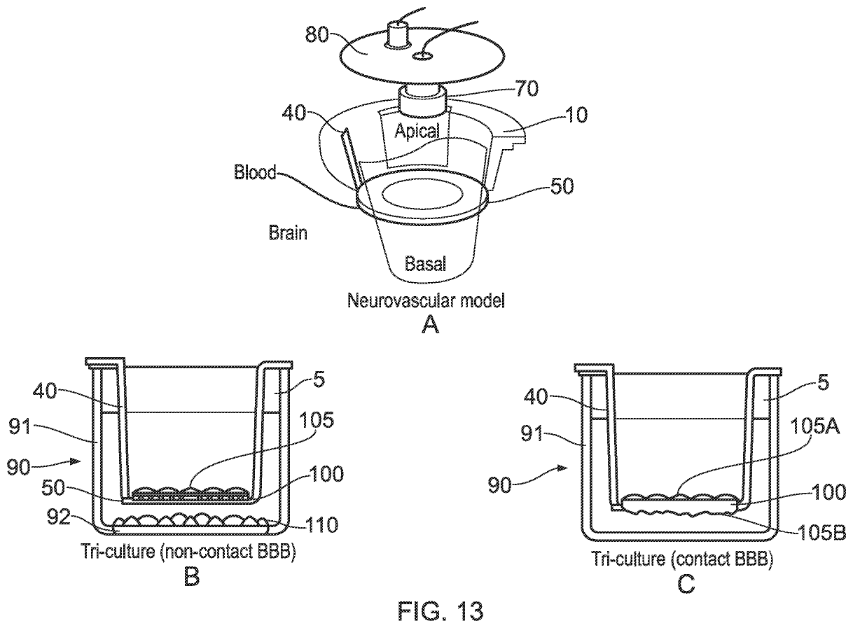

As shown in FIG. 13C, the insert (5) may include a microelectrode array (MEA). The MEA may be attached to the lower surface of the porous scaffold membrane. For example, an MEA may be a flexible MEA that is adhered to the lower surface of the porous scaffold membrane. In other examples, the MEA may be attached to the lower surface of the porous scaffold membrane by the use of attachment elements that can be partially inserted into the membrane to secure the MEA to the porous scaffold membrane.

An insert as described herein may be used as part of an apparatus (also referred to as a device) for dynamically monitoring a 3-dimensional cell culture. The insert is disposed into a support device that is suitable for culturing cells such as a well plate. The support device may have a plurality of wells, such as a multiwell plate.

When the support device has a plurality of wells, each well may have an insert disposed therein. Each well is configured to receive an insert. For example, the well and insert may each have shapes and/or dimensions so that the insert can fit inside the well.

The insert is disposed in the well so that the porous scaffold membrane is suspended above a lower surface (i.e. the bottom) of a well and separates the well (i.e. acts as a barrier) into an upper (apical) and lower (basal) portion. In any of the arrangements described herein the insert may be disposed in the well so that the upper and lower portions of the well are isolated from each other. That is to say that the upper and lower portions are not in communication with each other apart from via the porous scaffold membrane and any cell cultures thereon and/or therein. The upper and lower portions may be isolated by the use an adhesive. For example, application of an air tight and/or liquid tight adhesive to a portion of the outer surface of the insert or to a portion of the wall of the well. The upper and lower portions of the well may also be isolated by use of a sealing O-ring or other suitable structure or element. In such an arrangement it will be understood that cell culture medium is placed into the lower portion of the well prior to the insert being disposed therein.

The apparatus may also include a counter electrode as described herein. The counter electrode may be provided in a basal (lower) portion of the well. For example, below an insert when the insert is disposed in the well.

In another example the counter electrode is integrated with a lid of the support device that is configured to cover the well and the insert disposed therein. As described herein with reference to FIG. 8 the counter electrode is integrated with a lid (80) that covers the apical portion of the insert and/or the well. The lid (80) includes an opening (81) to allow connection of the counter electrode to other electronic devices via a connector (71). FIGS. 9 and 10 show an example of a lid with an integrated counter electrode.

The counter electrode includes a connector (71). The connector may be any suitable connecter adapted to connect the working electrode to other external electrical devices, such as cables or wires. Shown in FIGS. 9 and 10, the connector includes a holder (73) that is positioned within the opening (81) in the lid. The holder (73) may be any suitable holding device that can maintain the counter electrode in place. For example, the holder may be a luer cap and plug device. The connector shown in FIGS. 9 and 10 includes a metal pin (72) which is inserted into the holder (73) via an opening in extending through the centre of the holder (73). The metal pin may be any material that is capable of conducting electricity such as gold. The metal pin extends through the holder and through the opening (81).

The counter electrode includes an electrode (74) that has a protrusion (75) extending from a first surface. The protrusion (75) extends from the first surface of the electrode into opening of the holder so that the electrode is in electrical contact with the pin.

The electrode (74) is secured in place by a connection with the pin and/or the holder. The connection may be made by a fastening device, such as a crimp or by adhesive.

The electrode (74) is secured so that, in use, the electrode (74) is positioned within a upper (apical) portion of an insert. The electrode (74) is positioned so that in use it may be submerged in liquid, such as cell culture medium, in order to provide an electrical connection between the electrode (74), the porous scaffold membrane (50) and the working electrode (40). Other suitable arrangements for positioning the counter electrode in an upper portion of the insert when disposed in a support device, such as an upper portion of a well, will be known.

With reference to FIG. 8, the lid (80) and counter electrode (70) may be positioned over the top of an insert (10) when the insert is disposed within a support device (90).

The lid may be a lid of a support device that includes a number of wells (91), such as a multi-well plate with an insert in each well. As such, the lid may include a plurality of integrated counter electrodes each corresponding to each well, so that each well has a counter electrode positioned within an apical portion of the insert when the insert is disposed within a well and the lid is positioned over the support device so as to cover the insert and/or well.

As such, the lid and counter electrode integrated therein may be considered to be part of the support device in some examples.

In an alternative example, the counter electrode may be placed at a position remote to the well in which the insert is disposed. For example, in the case of a multiwell plate, the counter electrode may be placed in a well adjacent to the well in which the insert is disposed. In such an arrangement, the counter electrode may be submerged in an electrolyte and connected to a connector, such as a connector as described herein, so that the counter electrode, porous scaffold membrane (and any cells thereon and/or therein), and working electrode are in electrical communication when in use. In some examples, the counter electrode is connected to a connector including a wire that extends from the counter electrode to a connector positioned in or above the well in which the insert is disposed in. In some examples, the wire is submerged in an electrolyte. In some examples, the adjacent well may be connected to the well with the insert disposed therein. For example, via a channel configured to allow the flow of electrolyte but prevent the flow of cells between the two wells.

The apparatus may also include a reference electrode. The reference electrode may also be integrated with the lid of the support device. A reference electrode is an electrode which has a stable and well-known electrode potential. The high stability of the electrode potential is usually reached by employing a redox system with constant (buffered or saturated) concentrations of each participant of the redox reaction. The reference electrode may be an aqueous reference electrode, such as a standard hydrogen electrode a normal hydrogen electrode (NHE), a reversible hydrogen electrode (RHE), a saturated calomel electrode, a copper-copper(II) sulfate electrode, a silver chloride electrode, a pH-electrode, a palladium-hydrogen electrode, a dynamic hydrogen electrode (DHE), or a mercury-mercurous sulfate electrode.

The reference electrode may be made from a shapeable or malleable material. For example, the reference electrode may be made from a wire. This may allow for a reference electrode of any suitable shape to be made. For example, the reference electrode may be a ring. Conventional reference electrodes contact the electrolyte at a single point and so only guarantee the potential at that point. By making use of, for example, a wire ring, the potential in a plane horizontal to the porous scaffold membrane may better approximate an equipotential. This may result in more uniform ion flux through the cells cultured on the porous scaffold membrane, with the measurement becoming a more faithful representation of the entire biological construct. For example, the reference electrode may be a stainless steel ring.

In some examples, the reference electrode is a quasi-reference electrode. Reference electrodes are used commonly in electrochemistry (from which Electrical Impedance Spectroscopy (EIS) is taken), however true reference electrodes are expensive, have large form factors and often make use of cytotoxic components. As such reference electrodes are not used in EIS for (long term, for example in cell culturing methods) biological applications. Quasi-reference electrodes (electrodes without a constant interfacial potential, such as Platinum) are known to electrochemistry, but are seldom used as they confound the measurement of REDOX reactions due to their uncontrolled interfacial potentials. In contradiction to the central aim of electrochemistry, the system and the phenomena measured that is described herein, in general, is purely non-Faradaic (in absence of REDOX reactions) and so quasi-reference electrodes can be used, in particular for EIS, which uses small oscillating currents. As such, the use of stainless steel as a low-cost polarizable material to make up the reference electrode is a viable option for use with the apparatus and systems described herein.

In some examples, with reference to FIG. 13A, the support device may include a further member (92) that is located on the bottom of the well (91). For example, the further member (92) may be an electrode array, such as micro-electrode array (MEA). The MEA may have cells deposited and maintained thereon, such as neuronal cells or neurons.

A micro-electrode array is a device that includes a plurality microelectrodes. Cells, such as cardiomyocytes or neurons, which are electrically active, may be cultured over the electrodes creating a cohesive network. The field potential or activity across an entire population of cells is recorded by the MEA.

Also provided herein is a kit of parts. The kit of parts may include an insert as described herein without a lid or counter electrode, a support device as described herein and a counter electrode as described herein.

The kit of parts may include an insert as described herein with a lid including an integrated counter electrode and a support device with a lid without an intergrade counter electrode.

The kit of parts may include an insert as described herein without a lid or counter electrode and a support device that includes a counter electrode and a lid. For example, a support device with a counter electrode integrated with a lid or a counter electrode separate from the lid.

The porous scaffold membranes described herein may be used for forming 3-dimensional (3D) cell cultures. 3D cell culture refers to an artificially created environment in which cells are permitted to grow and/or interact with their surroundings in all three dimensions. Unlike 2D environments (e.g. a Petri dish), a 3D cell culture allows cells in vitro to grow in all directions, therefore mimicking growth in vivo.

The porous scaffold membranes described herein may be used for forming 3D cell structures such as organoids or models of tissue structures. For example, tissues may be barrier tissues. Barrier tissues include lining of the mouth, nose, trachea, bronchi, lung, oesophagus, stomach, gut, peritoneum, bladder, urethra, penis, prostate, uterus, vagina, arteries, veins or capillaries. As such, examples of tissues and structures include lung tissue, the blood brain barrier (BBB), intestinal tissue, vascular tissue, neuronal tissue, nasal tissue, gastrointestinal tract tissue, genital tissue and/or kidney tissue.

The porous scaffold membranes described herein may be used to grow any cell type or combinations thereof. For example, the porous scaffold membranes described herein may be used to culture tissue specific cells such as neuronal cells, intestinal cells, gastric cells, hepatic cells, vascular cells, stromal cells, immune cells, urinary system cells, cardiac cells and/or connective tissue cells.

The porous scaffold membranes described herein may provide a spatial cell organization in 3D that mimics tissues. The porous nature of the porous scaffold membranes allows for the flow of nutrients and/or compounds into and out of the porous scaffold membranes, creating a favourable environment for cell growth. Cells are able to attach, proliferate and differentiate within and/or on the porous network of the porous scaffold membrane.

The porous scaffold membrane may be seeded with a first cell type. The first cell type may be cultured using standard culturing methods prior to being used to seed the porous scaffold membrane. For example, the first cell type may be cultured in a suitable flask containing cell culture media to provide a first cell culture comprising the first cell type. The first cell culture may comprise the first cell type at a concentration suitable for allowing infiltration and attachment of cells to the porous scaffold membrane. For example, of at least 1×10³ cells/ml. In some examples, the first cell culture may comprise the first cell type at a concentration of at least 5×10⁶. The number of cells used for seeding the first cell type may be dependent on the type of cells and may be known or determined by standard methods.

The porous scaffold membrane may be soaked in the same or other suitable culture medium prior to seeding. Soaking the porous scaffold membrane in culture medium may help to allow for protein adhesion upon seeding. The porous scaffold membrane may be dried after soaking in cell culture medium.

The porous scaffold membrane may be submerged in the first cell culture in order to allow the first cell type to penetrate into the porous scaffold membrane. Penetration may be achieved by capillary action. The porous scaffold membrane may then be maintained within the first cell culture for a period time, for example at least 1 hour, at a temperature of around 37° C. and 5% CO₂ to allow the first cell type to attach to the porous scaffold membrane and spread throughout the porous scaffold membrane, thereby seeding the porous scaffold membrane.

Once the first cell type has been seeded into the porous scaffold membrane, the seeded porous scaffold membrane may be maintained by storing the seeded porous scaffold membrane in culture medium.

The first cell type may include connective tissue cells and/or stroma cells.

For example, connective tissue cells include fibroblasts, mast cells, plasma cells, macrophages, adipocytes, and/or leukocytes.

For example, stromal cells include endothelial cells, fibroblast cells, neuronal cells, mast cells, epithelial cells, myocardial cells, hepatic cells, pancreatic islet cells, tissue stem cells, mesenchymal stem cells and/or smooth muscle cells.

Fibroblasts are the most common cell type of connective tissue. Fibroblasts produce tropocollagen, which is the forerunner of collagen, and ground substance, an amorphous gel-like matrix that fills the spaces between cells and fibres in connective tissue. Fibroblasts also secrete collagen proteins that are used to maintain a structural framework for many tissues.

Mast cells are long-lived tissue-resident cells with an important role in many inflammatory settings including host defence to parasitic infection and in allergic reactions. Mast cells are located at the boundaries between tissues and the external environment. Mast cells mediate inflammatory responses such as hypersensitivity and allergic reactions. They are scattered throughout the connective tissues of the body, especially beneath the surface of the skin, near blood vessels and lymphatic vessels, within nerves, throughout the respiratory system, and in the digestive and urinary tracts. Mast cells store a number of different chemical mediators including histamine, interleukins, proteoglycans (e.g., heparin), and various enzymes in coarse granules found throughout the cytoplasm of the cell.

Plasma cells are short-lived antibody-producing cells derived from a type of B cells. B cells differentiate into plasma cells that produce antibody molecules closely modelled after the receptors of the precursor B cell.

Macrophages are constituents of the reticuloendothelial system (or mononuclear phagocyte system) and occur in almost all tissues of the body. In some instances, macrophages are fixed in one place within tissues, such as in the lymph nodes and the intestinal tract. In other cases, they may wander in the loose connective-tissue spaces. As a group they have the ability to ingest other cells, infectious agents, and many other microscopic particles, including certain dyes and colloids. Macrophages, by ingesting and processing foreign particles, play a key role in rendering them recognizable by lymphocytes, which determine the specificity of the immune response.

Adipose cells, also called adipocytes or fat cells, are connective-tissue cells specialized to synthesize and contain large globules of fat. There are two types of adipose cells: white adipose cells contain large fat droplets, only a small amount of cytoplasm, and flattened, noncentrally located nuclei; and brown adipose cells contain fat droplets of differing size, a large amount of cytoplasm, numerous mitochondria, and round, centrally located nuclei. The colour of brown adipose is attributed to its relatively high density of mitochondria and its extensive vascular supply. The main chemical constituents of adipose cell fat are triglycerides, which are esters made up of a glycerol and one or more fatty acids, such as stearic, oleic, or palmitic acids. Enzymes contained in adipose cells specialize in the hydrolysis of triglycerides in order to generate fatty acids and glycerol for physiological processes. The main reservoir of fat in the body is the adipose tissue beneath the skin, called the panniculus adiposus. There are also deposits of fat between the muscles, among the intestines and in their mesentery, around the heart, and elsewhere. One function of these deposits is to act as soft elastic padding between the various organs.

White blood cells, also called leukocytes or white corpuscles, are a cellular component of the blood that lacks haemoglobin, has a nucleus, is capable of motility, and defends the body against infection and disease by ingesting foreign materials and cellular debris, by destroying infectious agents and cancer cells, or by producing antibodies. Although white cells are found in the circulation, most occur outside the circulation, within tissues. On the basis of their appearance under a light microscope, white cells are grouped into three major classes-lymphocytes, granulocytes, and monocytes. Lymphocytes, which are further divided into B cells and T cells, are responsible for the specific recognition of foreign agents and their subsequent removal from the host. B lymphocytes secrete antibodies, which are proteins that bind to foreign microorganisms in body tissues and mediate their destruction. Typically, T cells recognize virally infected or cancerous cells and destroy them, or they serve as helper cells to assist the production of antibody by B cells. Also included in this group are natural killer (NK) cells, so named for their inherent ability to kill a variety of target cells. In a healthy person, about 25 to 33 percent of white blood cells are lymphocytes.

Endothelial cells are the main type of cell found in the inside lining of blood vessels, lymph vessels, and the heart. Endothelial cells form a thin layer of simple squamous cells that line the inner surface of blood vessels and lymphatic vessels. Endothelial cells in direct contact with blood are called vascular endothelial cells, whereas those in direct contact with lymph are known as lymphatic endothelial cells. Capillary endothelial cells are found at the blood brain barrier. These cells are found at the tip of the capillaries that interface with the brain and are specialised endothelial cells that form a highly selective barrier that allows nutrients and gases into the brain while keeping other molecules from entering the brain.

Neural cells refers broadly to cells associated with the central nervous system (CNS) of an organism, for example, neurons, glial cells, and precursor cells. As used herein, neural cells may be cells that are isolated or derived from neural tissue, as well as any cell, regardless of origin, having at least an indication of neuronal or glial phenotype, such as staining for one or more neuronal or glial markers or which will differentiate into cells exhibiting neuronal or glial markers.

Neuronal cells are polar cells of the vertebrate nerve system which are specialized for the transmission of nerve impulses. Such cells typically display neuronal cell structure and express at least one neuronal marker. Examples of such markers include, but are not limited to neuronal and dopaminergic markers examples of which include, but are not limited to, Peripherin, Choline Acetyltransferase [ChAT], Chromogranin A, DARPP-32, GAD65, GAD67, GAP43, HuC, HuD, Alpha internexin, MAPS, MAP-2 A&B, Nestin, NeuN, Neurofilament L, M, H, Neuron-Specific Enolase (gamma-NSE), P75, low affinity NGF receptor, Peripherin, PH8, Protein Gene Product 9.5 (PGP9.5), Serotonin Transporter (SERT), Synapsin, Tau, Thy-1, TrkA, Tryptophan Hydroxylase (TRH) Beta Ill Tubulin, TUC-4 (TOAD/Ulip/CRMP) Tyrosine hydroxylase (TH) Vanilloid Receptor Like Protein 1 (VRL-1), Vesicular GABA Transporter (VGAT) Vesicular Glutamate Transporter 1 (VGLUT1; BNPI) and VGLUT2.

Glial cells refers to all types of central nerve system (CNS) cells that cannot receive or transmit nerve signals. Generally, “glial cells” include astrocytes, oligodendrocytes, ependymal cells and their precursors.

Epithelial cells are cells that form the external surface of the body and lining the organs, cavities and mucosal surfaces. Epithelial cells are specialized cells that provide diverse functions for the tissue and/or the systemic needs of a host. They are recognized by their ability to migrate as precursors or immature cells; with maturation, they become stationary and form layers of squamous or cobblestone-like or columnar polarized cells with apical, basal and lateral sides, and are bound to each other by an assortment of junctions (connexins, tight junctions, adherens junctions, gap junctions, desmosomes). Their expansion potential is indicated by the diameter of a colony (not by its density). Mature epithelial cells provide diverse functions such as secretion of specialized products or of contributions to metabolism (hepatocytes, cholangiocytes), detoxification (hepatocytes), production of enzymes (acinar cells), production of endocrine factors (e.g. islets or other endocrine cells), electrical activity (neuronal cells), and absorption (intestinal cells). Examples of epithelial cells include keratinocytes, intestinal epithelial cells, oral epithelial cells and corneal epithelial cells.

For example, intestinal epithelial cells can include enterocytes, Goblet cells, enteroendrocrine cells, Paneth cells, microfold cells, cup cells, tuft cells, progenitors thereof, and stem cells.

Myocardial cells are cells of the muscular tissue of the heart (the myocardium). Examples of myocardial cells include myocardial stem cells, cardiomyocytes and pacemaker cells.

Hepatic cells or hepatocytes refer to cells that have characteristics of epithelial cells obtained from liver. Hepatic cells include hepatic parenchymal cells that perform the major functions of the liver including production of bile and metabolism of various substances.

“Smooth muscle” refers to a muscle system that constitutes the walls of the internal organs of the human body and is a tissue that controls the movement of various internal organs (e.g., the gastrointestinal tract, blood vessels, bladder or uterus). Smooth muscle cells refer to cells isolated from such tissue. Characteristics of smooth muscle cells include a histological morphology (under light microscopic examination) of a spindle shape with an oblong nucleus located centrally in the cell with nucleoli present and myofibrils in the sarcoplasm.

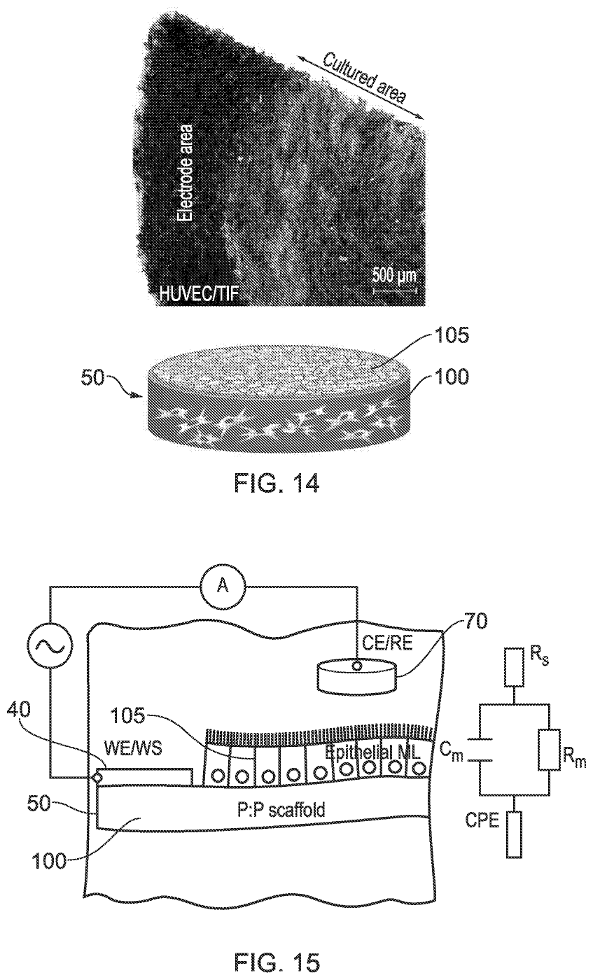

Once the porous scaffold membrane has been seeded with the first cell type, that is to say that the first cell type is disposed within the porous scaffold membrane, at least one second cell type is seeded onto one or more surface of the porous scaffold membrane (for example see FIG. 14). For example, a second cell type may be seeded onto one surface of the porous scaffold membrane and at least one further second cell type seeded onto an opposing surface of the porous scaffold membrane (for example see FIG. 13). In another example, one or more surfaces of the porous scaffold membrane may be seeded with a plurality of second cell types, for example, 2, 3, 4 or more cells types seeded onto a single surface.

The second cell type may be cultured using standard culturing methods prior to being used to seed the porous scaffold membrane. For example, the second cell type may be cultured in a suitable flask containing cell culture media to provide a second cell culture comprising the second cell type. The second cell culture may comprise the second cell type at a concentration suitable for allowing attachment of cells to the porous scaffold membrane. For example, of at least 1×10³ cells/ml. In some examples, the second cell culture may comprise the second cell type at a concentration of at least 5×10⁶. The number of cells used for seeding the at least one second cell type may be dependent on the type of cells and may be known or determined by standard methods.

The porous scaffold membrane may be submerged in the second cell culture in order to allow the second cell type to adhere to the porous scaffold membrane. The porous scaffold membrane may then be maintained within the second cell culture for a period of time, for example at least 1 hour, at a temperature of around 37° C. to allow the second cell type to attach to the surface of porous scaffold membrane and spread across the surface of the porous scaffold membrane, thereby seeding the surface of porous scaffold membrane.

Once the second cell type has been seeded on to the porous scaffold membrane, the seeded porous scaffold membrane comprising the first and second cell type may be maintained by storing the seeded porous scaffold membrane in culture medium.

The second cell type or types may be selected depending on the tissue or system which the porous scaffold membrane is being used to mimic.

In general, the second cell type or types comprise epithelial and/or endothelial cells. In some examples the second cell type includes epithelial cells. In some examples the second cell type includes endothelial cells. In some examples the second cell type includes epithelial and endothelial cells.

The second cell type may form a monolayer of cells on one more surfaces of the porous scaffold membrane. A monolayer refers to a single layer of cells. The second cell type may include multiple layers of cells.

Endothelial cells include lymphatic endothelial cells and vascular endothelial cells. Examples of endothelial cells include, umbilical vein endothelial cells, dermal microvascular endothelial cells, aortic endothelial cells, coronary artery endothelial cells, dermal blood endothelial cells, cardiac microvascular endothelial cells, brain microvascular endothelial cells, iliac artery endothelial cells, lung microvascular endothelial cells, aortic valve endothelial cells, and/or valvular interstitial cells.

Epithelial cells may include epithelial cells derived from any organ or part of the body such as the gastrointestinal tract, airways, reproductive system, kidneys, liver, or nervous system. For example, epithelial cells may include one or more of respiratory epithelial cells, gastric epithelial cells, intestinal epithelial cells, neural epithelial cells, respiratory epithelial cells, mesothelium cells, reproductive epithelial cells, germinal epithelial cells, sensory epithelial cells, olfactory epithelial cells, corneal epithelial cells, urinary epithelial cells and/or urothelium cells.

The epithelial cells may be of any structure such as, columnar, pseudostratified (an epithelium that, though comprising only a single layer of cells, has its cell nuclei positioned in a manner suggestive of stratified epithelia), squamous (flattened and scale-like cells), and/or cuboidal.

For example, for a general endothelial cell model system, the second cell type may include human umbilical vein endothelial cells (HUVEC). HUVECs are primary cells isolated from the vein of the umbilical cord. They are a model system for studying endothelial cell function, with applications including hypoxia, inflammation, oxidative stress, response to infection, and both normal and tumor-associated angiogenesis.

For example, in the case of an intestinal model system, the second cell type may include enterocytes, goblet cells, enteroendocrine cells, Paneth cell microfold cells, cup cells, and/or tuft cells.

For example, for a vascular model system, the second cell type may include endothelial cells. In some examples, the second cell type may include smooth muscle cells.

For example, for a epidermal model, the second cell type may include keratinocytes, melanocytes, Langerhans cells, Merkel cells, and/or inflammatory cells.

For example, for a respiratory epithelial model (such as lung, trachea alveoli, or bronchiole epithelial model), the second cell type may include ciliated cells, undifferentiated columnar cells, secretory cells, Clara cells, alveolar type I cells, alveolar type II cells, cartilage cells, mucus glands, neuroendocrine cells, goblet cells, alveolar epithelial cells, bronchial epithelial cells, pulmonary artery endothelial cells, bronchial smooth muscle cells, tracheal smooth muscle cells, alveolar macrophages and/or basal cells.

For example, as shown in FIG. 13C, for a blood brain barrier model, the second cell type may include capillary endothelial cells seeded onto one surface of the porous scaffold membrane and neurons seeded onto an opposing surface of the porous scaffold membrane.

Cells and cell types that may be used with the porous scaffold membrane may be derived from any source. For examples, cells may be animal cells. For example, the first cell type and/or the second cell type may be animal cells. For example, the first cell type and/or the second cell type may be mammalian cells. In some examples, the first cell type and/or the second cell type are human cells.

In some examples the first and/or second cell types may be prokaryotic cells. Such as bacterial cells.

In some examples, the at least one second cell type may be an organoid or organoid cell culture. “Organoid” refers to a heterogeneous 3D agglomeration of cells that recapitulates aspects of cellular self-organization, architecture and signalling interactions present in a native organ. The term “organoid” includes spheroids or cell clusters formed from suspension cell cultures. Such organoids may be derived from induced pluripotent stem cells and/or derived from tissue specific stem cells (also referred to as somatic or adult stem cells). For example, organoids may be derived from embryonic stem cells, somatic stem cells, germ stem cells, epidermal stem cells, or tissue-specific stem cells. Examples of tissue-specific stem cells include, but are not limited to, neural stem cells, keratinocyte stem cells, renal stem cells, embryonic renal epithelial stem cells, embryonic endodermal stem cells, hepatocyte stem cells, mammary epithelial stem cells, bone marrow-derived stem cells, skeletal muscle stem cells (satellite cells), limbal stem cells, hematopoietic stem cells, mesenchymal stem cells, peripheral blood mononuclear progenitor cells, splenic precursor stem cells, intestinal stem cells and/or oesophageal stem cells. Organoids may also be derived from one or more samples taken from a subject, such as a human subject. For example, organoids may be derived from samples taken from specific organs tissue such as lung tissue, oesophageal tissue, heart tissue, muscle tissue or intestinal tissue. For example, samples may be biopsies taken from a subject.

In some examples, at least one further cell type (such as at least one third, fourth or fifth cell type) may be added or included in the culture medium in which a 3D cell culture is maintained in. For example, at least one further cell type may be added into the culture medium that is in a basal and/or apical portion of a well plate having an insert disposed therein including a 3D cell culture as described herein. In some examples, the further cell type may be bacterial cells. In some examples, the bacterial cells may form a biofilm. Such examples, may allow the formation and/or effects of biofilms on model tissue systems to be studied. In some examples, the further cell type may include cancer cells.

In some examples, the at least one further cell type may include an organoid or organoid culture as described herein.

Culture medium that may be used for culturing the first and/or second cells and/or 3D cell cultures formed therefrom may be any suitable cell culture medium and may be selected depending on the types of cells being cultured. Examples of culture medium that may be used include minimal essential medium (MEM, Sigma, St. Louis, Mo); Dulbecco's modified Eagle medium (DMEM, Sigma); Ham F10 medium (Sigma); Cell culture media (HyClone, Logan, Utah); RPMI-1640 culture media (Sigma); and chemical-defined (CD) culture media (which are formulated for individual cell types), such as CD-CHO culture media (Invitrogen, Carlsbad, Calif). The culture solution described above can be supplemented with auxiliary components or contents as needed. This includes any component of the appropriate concentration or amount required or desired.

The culture solution described above can be supplemented with auxiliary components or contents as needed. The culture medium may include one or more additives such as antibiotics, proteins, amino acids and/or sugars.

“Medium” and “cell culture medium” refer to a nutrient source used for growing or maintaining cells. As is understood by a person of skill in the art, the nutrient source may contain components required by the cell for growth and/or survival or may contain components that aid in cell growth and/or survival. Vitamins, essential or non-essential amino acids, trace elements, and surfactants (e.g., poloxamers) are examples of medium components. Any media provided herein may also be supplemented with any one or more of insulin, plant hydrolysates and animal hydrolysates.

In some examples, cells may be cultured or initially cultured in a proliferation medium. Proliferation medium may be a medium comprising a source of nutrients, such as vitamins, minerals, carbon and energy sources, and other beneficial compounds that facilitate the biochemical and physiological processes occurring during expansion or proliferation of cells. The proliferation medium may comprise one or more carbon sources, vitamins, amino acids, and inorganic nutrients. Representative carbon sources include monosaccharides, disaccharides, and/or starches. For example, the proliferation medium may contain one or more carbohydrates such as sucrose, fructose, maltose, galactose, mannose, and lactose. The proliferation medium may also comprise amino acids. Suitable amino acids may include amino acids commonly found incorporated into proteins as well as amino acids not commonly found incorporated into proteins, such as argininosuccinate, citrulline, canavanine, ornithine, and D-steroisomers. The proliferation medium may also comprise proteins such as foetal bovine serum albumin. The proliferation medium may also comprise antibiotics.

For example, the proliferation medium may be Dulbecco's Modified Eagle's Medium (DMEM) that may include 10% (V/V) filter sterilized foetal bovine serum and 1% (V/V) penicillin/streptomycin solution.

The cells may be cultured in a differentiation medium. The cell culture media may be changed from an initial culture medium to a second cell culture medium. The second cell culture medium may be a differentiation medium. Differentiation medium refers to a medium designed to support the differentiation of cells, that is, supporting the process of a cell changing from one cell type to another. The differentiation medium may include one or more amino acids, antibiotics, vitamins, salts, minerals, or lipids. The differentiation medium may include at least one carbon source such as a sugar. For example, glucose. The differentiation medium may include one or more proteins, amino acids or other additional acids. In some examples the differentiation medium may be high-glucose DMEM (97%) supplemented with 2% horse serum and 1% penicillin/streptomycin solution. In other examples the differentiation medium may include Wnt, ROCK inhibitor (or Y) or B27.

“Culturing” a cell refers to contacting a cell with a cell culture medium under conditions suitable to the viability and/or growth and/or proliferation of the cell.

The porous scaffold membrane may be seeded with the first cell type and then placed into an insert as described herein. The insert may then be disposed in a support device having culture medium therein. The second cell type may then added to the membrane and seeded onto the porous scaffold membrane including the first cell type.

In other examples, the porous scaffold membrane may be placed into an insert as described herein and then seeded with the first cell type and subsequently with the second cell type.

In another example, the porous scaffold membrane may be seeded with the first and second cell types and then placed into an insert as described herein.

It will be understood that the methods of seeding and culturing and culture media may be selected depending on the specific cells that are being used. The timing and order of the steps of seeding the porous scaffold membrane and its placement into an insert may also be selected based on the specific cell type used.

The apparatus described herein may be used in a number of methods of dynamically monitoring a 3D cell culture formed on a porous scaffold membrane as described herein.

Dynamically monitoring is used to refer to monitoring of living cells in real time. Monitoring as used herein may include detecting, analysing and/or recording properties of a 3D cell culture, such as electrical properties.

Electrical properties may include voltage, current, resistance, impedance, capacitance, and/or conductivity of a 3D cell culture.

The methods include providing an insert as described herein including a porous scaffold membrane as described herein and a support device. A 3D cell culture as described herein is formed on the porous scaffold membrane.

The insert including the 3D cell culture formed onto and within the porous scaffold membrane is disposed into the support device, for example the insert is disposed into a well of a well plate.

The insert is disposed so that at least part of the 3D cell culture is immersed in liquid such as cell culture medium. In some examples, such as those shown in FIGS. 8 and 11, both surfaces of the porous scaffold membrane and the 3D cell culture seeded thereon and therein are submerged in the liquid.

In addition, the working electrode is disposed in the liquid and the counter electrode is immersed within the liquid. As described herein, the counter electrode may be placed in the bottom of a well below the insert or may positioned above the insert, for example by integration with a lid of the inert or support device as described herein. Alternatively, the counter electrode may be placed in a well containing a liquid adjacent to the well having an insert disposed therein and connected via a connector to the insert.

The liquid acts as an electrolyte in order to provide an electrical connection between the porous scaffold membrane, the working electrode and counter electrode. The liquid may be any suitable liquid that may support and maintain the cells of the 3D cell culture such as cell culture medium as described herein.

The culture medium may include one or more additives such as antibiotics, proteins, amino acids and/or sugars.

In some examples, such as that shown in FIG. 12, one surface of the porous scaffold membrane is immersed in the liquid and the second surface is open to the air. In such an arrangement the counter electrode is located below the insert, submerged in the liquid in order to allow for the counter electrode to be in electrical contact with the working electrode when in use via the liquid.

Once immersed in the liquid, an electrical signal may be applied to the porous scaffold membrane and the 3D cell culture seeded thereon via the working electrode and the counter electrode. Voltage may be applied by any suitable means such as by using a source meter connected to the counter electrode and working electrode.

The electrical signal may be an alternating current, direct current and/or a voltage.

The electrical properties of the 3D cell culture are then monitored by use of a monitoring device connected to the working electrode and counter electrode which is configured to record electrical properties as described herein and changes thereof.

The monitoring device may be any suitable device known in the art that is for monitoring electrical properties such as conductance, impendence, voltage and/or current. For example, a potentiostat may be used to monitor electrical properties of the porous scaffold membranes and/or the cells cultured therein and/or therein.

As shown in FIG. 15, the counter electrode (70), working electrode (40), liquid and 3D cell culture (110) form an electrical circuit of components in series. Thus allowing monitoring of electrical properties of the 3D cell culture.

The apparatus and methods of using the apparatus described herein may be used to monitor trans-Epi/Endothelial Electrical Resistance (TEER). TEER may be used for assaying barrier tissue integrity, as well as permeability.

TEER involves applying an AC electrical signal across electrodes, such as the working electrode and counter electrode, placed on both sides of a cellular layer, such as the 3D cell culture described herein, and measuring voltage and current to calculate the electrical resistance of the 3D cell culture.

TEER may be determined by impedance based methods such as electrical impedance spectroscopy (EIS). EIS involves measurements taken across a range of frequencies and plotting resulting impedance as a function of frequency. Since capacitive reactance (the effective resistance provided by a capacitor) decreases as frequency increases while resistance is frequency independent, impedance spectrum can show the contributions of specific capacitances to the overall impedance. As such, EIS measurements carried out throughout the culturing and/or maintenance of the 3D cell culture, may allow determination of electrical signatures from different cell types, as well as assessment of cell growth and extraction of barrier function parameters.

EIS may be carried out using an insert that is in an electrode arrangement and is therefore operated as an electrode. An electrode arrangement includes the use of a single working electrode as shown in FIGS. 7, 8, 12 and 15.

As depicted in FIG. 11, the insert may include two working electrodes positioned on opposing surfaces of the porous scaffold membrane. With such an arrangement the apparatus may be operated as a transistor.

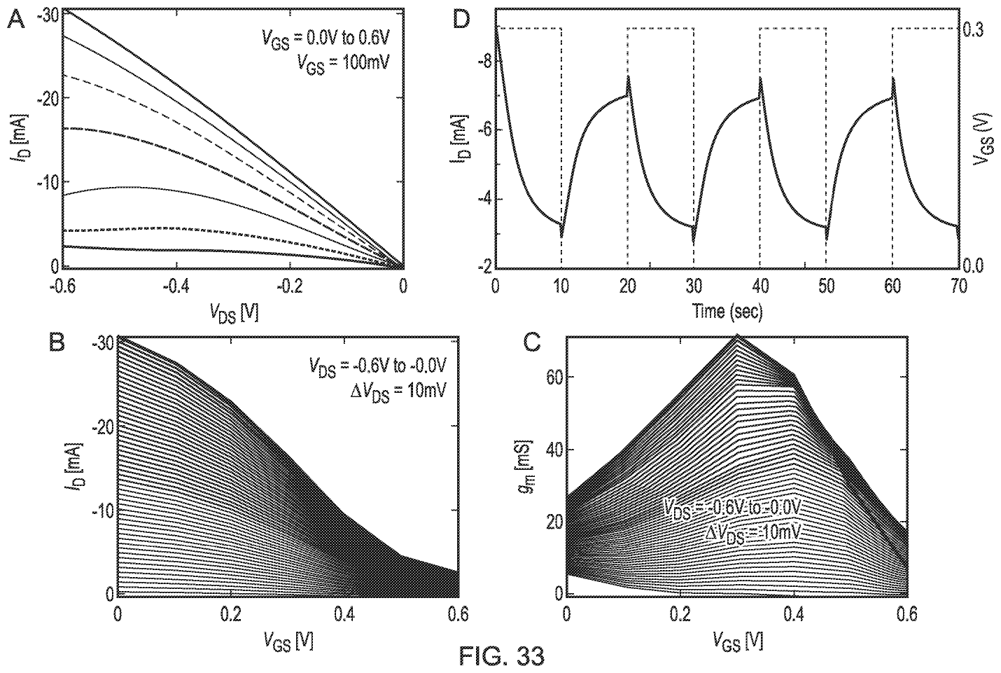

Transistor operation is controlled by a voltage (gate voltage) applied to the counter electrode (gate electrode of transistor) and between the first and second working electrodes (that act as drain and source electrodes of the transistor). The gate voltage controls the ion injection into the conducting polymer of the porous scaffold membrane which in turn alters the redox state of the porous scaffold membrane. At the same time the application of a voltage to the source and drain electrodes causes current flow at the porous scaffold membrane which acts as a channel of the transistor. The use of a p-type doped or oxidized based channel material (such as PEDOT:PSS) enables a depletion mode of operation; at zero gate voltage, current flows in the channel (the porous scaffold membrane) of the transistor. By applying a positive gate voltage, cations from the electrolyte (e.g. cell culture medium) are injected into the channel and anions (for example PSS in PEDOT:PSS conducting polymer) are compensated, causing a decrease in the number of holes and thus a decrease in the current. Thus, upon depletion operation the transistor shifts from an on to an off state. Transistor operation of the apparatus as described herein may be used to measure current that flows in the channel (porous scaffold membrane). This may be used to derive the transconductance which expresses the magnitude of amplification in drain current for a given change in gate potential and/or the magnitude of the change in drain current for a given change in gate potential. Additional, measurements that transistor operation may allow for include pulsed characteristics for measuring reversibility, operation stability and current response to a specific gate voltage profile. For example, see FIG. 33.

As detailed above the apparatus and methods described herein may be used to monitor and/or determine TEER of cells. TEER may be monitored and/or determined in order to monitor changes that may occur to cells of the 3D cell culture. For example, the apparatus and methods described herein may be used to monitor cellular response to different molecules, cellular response to different stimuli, transport of molecules through endothelium and/or epithelium cells, cell migration, cell attachment, cellular response to pathogens, cellular response to toxins, cellular response to other cells, cell growth and/or cell differentiation. For example by exposing the 3D cell culture to an agent of interest (e.g. drug, pathogen, other cells), for example by adding the agent of interest to the culture medium and monitoring the electrical properties of the cells.

As such the apparatus and methods described herein may be used for drug discovery and for research into effects (toxicity and/or efficacy) that drugs may have on certain cell types. For example by exposing the 3D cell culture to a drug, for example by adding the drug to the culture medium, and monitoring the electrical properties of the cells.

The apparatus and methods described herein may be used to monitor transport of molecules, such as drugs, through epithelial and/or endothelial cells. In addition the apparatus and methods described herein may be used to study cell migration and movement. In addition the apparatus and methods described herein may be used to study cell proliferation. In addition the apparatus and methods described herein may be used to study cell differentiation. In addition the apparatus and methods described herein may be used to study cell interactions and response to pathogens.

In some examples, the apparatus and methods described herein may be used to study specific cell structures or organoids. For example, the insert as shown in FIG. 11 may be seeded with a second cell type that comprises airway epithelial cells as described and provide a model system of the airway. The counter electrode may be located in a well below the insert thus allowing the airway epithelium to be exposed to the air, while the lower surface and body of the porous scaffold and connective and/or stromal cells seeded therein along with the working electrode may be immersed in liquid, mimicking the environment of the respiratory tract. Such a model may be used for the study of virus or bacteria interactions with such cells.

In another example, apparatus and methods described herein may be used to study the blood brain barrier. For example, the insert and apparatus as shown in FIGS. 13B and 13C. In FIG. 13B a third cell type (110), such as neuronal cells or neurons may be cultured on the bottom surface of a well that comprises a micro-electrode array (92). The first cell type (100) located within the porous scaffold membrane (50) may be glial cells such as astrocytes and the second cell type (110) located on the upper surface of the porous scaffold membrane may be brain capillary endothelial cells. The MEA may be used to record neural signals thus allowing the apparatus to monitor electrical properties of the epithelial cells and glial cells in response to such neural signals.

In FIG. 13C, the first cell type (100) may be glial cells such as astrocytes and the second cell type (110A) located on the upper surface of the porous scaffold membrane may be brain capillary endothelial cells and the second cell type located on the lower surface of the porous scaffold membrane may be neuronal cells or neurons. An MEA may be incorporated into the porous scaffold membrane in order to record neural signals and/or to stimulate neuronal cells.

When a transistor arrangement as shown in FIG. 11, culturing a 3D cell culture in a transistor device can provide qualitative and/or quantitative data of cell coverage, tissue formation and/or cell health. Cell health refers to any parameters of cell function that lead to a perceived and/or quantifiable improvement in the viability of the cell and/or cell type.

For example, the presence of a cell monolayer on the porous scaffold membrane (channel) of the transistor forms an additional barrier that can inhibit and/or suppress the ion flow in the channel and alter the measurable electrical characteristics of the transistor (such as drain current, transconductance, threshold voltage, and/or switchability).

### EXAMPLES

Example 1—Fabrication of an insert and lid integrated counter electrode

### Apical Electrode Module

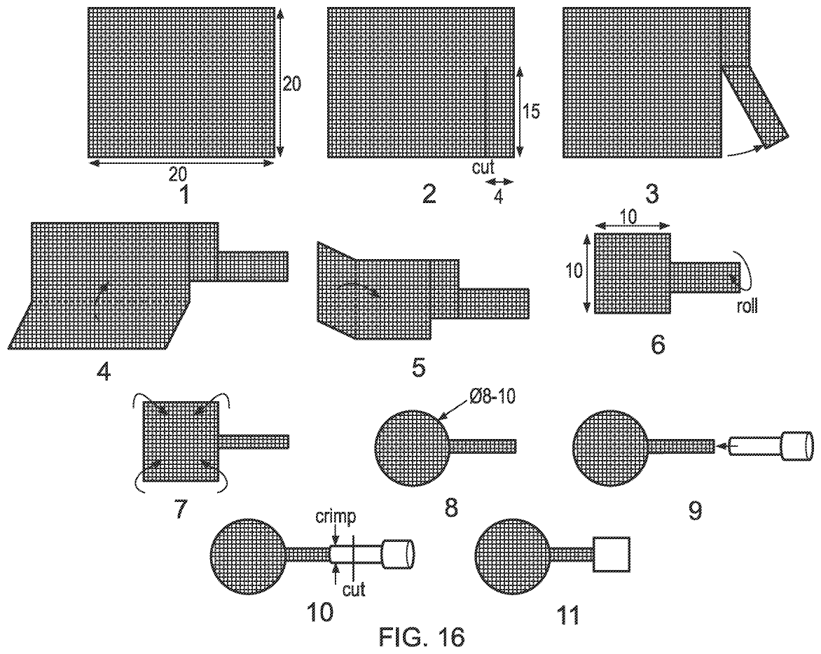

1. The complete counter electrode module is shown in FIGS. 9 and 10.
2. The lid of the 24 Well plate is modified by drilling a 5 mm hole in the centre of each well which is to be used (x n). a. The centre of the circular well is located by the intersecting-lines-perpendicular-to-chords method.
   b. The hole is drilled such that a male Luer slip plug fits firmly, without cracking the plate and without any gaps between plug and lid.
3. The male slip plug is modified by cutting the tapered portion of the plug to a final length of 7 mm. 4. A 0.8 mm hole is drilled in the centre of the blocked end of the male Luer plug.
a. The hole diameter is such that the gold pin (Buccaneer 400 Male Crimp Circular Connector) is inserted with a tight fit.
5. The modified male Luer plug is inserted into the modified well plate lid.
6. The Platinum (or stainless steel) mesh, is folded as illustrated in FIG. 16.
a. The final mesh construct is a circular mesh, four (4) layers thick, between 8 and
10 mm in diameter, with a rolled mesh protrusion, approximately 15 mm in length. On the end of the protrusion, is a square of crimped metal, approximately 2×2 mm.
   b. The square metal end piece is cut from a crimp connector, which is crimped onto the end of the mesh protrusion and trimmed.
7. The modified lid is inverted and each mesh projection is inserted into the modified Luer plug.
8. The CAPL dust cap is pressed into the plug such that the mesh projection is secured inside the Luer plug, while the mesh circle remains free.
   a. The mesh circle is folded over the basal end of the CAPL dust cap, such that the circle is parallel to the plane of the well plate lid.
9. The gold pin is inserted into the apical hole drilled into the Luer plug until significant resistance is felt.
10. The resistance between gold pin and mesh is measured using a multimeter.
   a. Acceptable values are in the region of 0.2Ω.
11. The CAPL dust cap is adhered onto the Luer plug, as well as the gold pin onto the Luer plug.

### Membrane Preparation

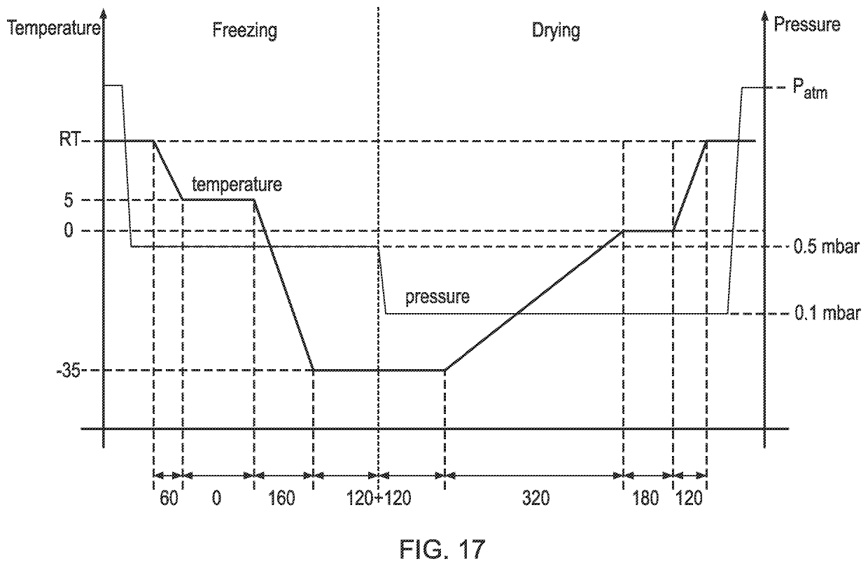

1. The membrane composition is prepared and stored at 5° C.
2. Immediately prior to lyophilization, the composition is ultrasonicated for 15 minutes
   a. Ice is added to the ultrasonication vat to maintain the temperature to <20° C.
3. Using a P1000 micropipette, 500 μl of the mixture is transferred into individual wells of a Well plate.
4. The well plate, without the lid, is placed into the lyophilization chamber and the programme depicted in FIG. 17 is run.
5. Once the lyophilization is complete, the scaffold is loosened from the sides of the wells.
(if using GOPS or other crosslinkers) The scaffold is placed into a well plate well and the well plate placed on a hot plate at 80° C. to allow the GOPS to crosslink for 8 hrs.
6. PDMS in a 10:1 ratio is poured into a petri dish to a depth of approximately 5 mm and allowed to cure overnight at ambient temperature.
7. An 8 mm biopsy punch is used to remove round sections of cured PDMS and each is placed in a well plate well, on top of a piece of tissue paper, to prevent to PDMS from adhering to the well plate base.
8. A small volume of uncured PDMS is placed onto the apical surface of the round PDMS base sections.
9. The crosslinked scaffolds are placed onto the PDMS bases so that the uncured PDMS adheres the basal surface of the scaffold to the apical surface of the PDMS base and are allowed to cure on a hotplate at 5° C. for 2 hrs.
10. Scaffolds are then cut using a vibratome to the desired thickness.

### Insert Preparation

The method described is based on modification of a Transwell™ insert available from Corning Inc.
1. The polycarbonate membrane at the base of the well is removed.
2. The top portion of the well, such that the well has a final height of
   i. 9 mm for Type 1 (Round type)
   ii. 11 mm for Type 2 (polygonal type)
3. PDMS is prepared at a 10:1 ratio.
4. Using the PDMS, the modified Transwell is glued into the top part of the CellCrown insert.
   a. cure overnight, or for >2 hrs on a hot plate at 5° C.
5. Gasket (membrane support member) and the Gold/Kapton connector are cut.
6. The module is then assembled:
   a. the scaffold is positioned such that it is concentric with the gasket and there are no gaps between the scaffold and the gasket centre hole.
   b. The Gasket/Scaffold are placed into the base part of the CellCrown, such that the scaffold is on the top and the gasket is in contact with the CellCrown.
   c. the Gold/Kapton contact is placed on top of the scaffold, such that the Gold side is in contact with the scaffold and is concentric with the scaffold
   d. The upper part of the CellCrown is then attached to the lower part, sandwiching the Gasket/Scaffold/Gold.

### Example 2-3D Organic Electronic Transmembrane Device for Tissue Hosting and Monitoring

Throughout the example, the terms “transmembrane”, “bioelectronic transmembrane” and “e-transmembrane” are used to refer to a porous scaffold membrane as described herein. The terms “device”, “3D e-transmembrane device”, “transmembrane device” and “e-transmembrane device” are used to refer to an apparatus as described herein.

A widely used cell culture setup for various in vitro applications (e.g., cell migration, toxicology, drug/nutrient transport) is the Transwell® insert, which allows the growth of cells on porous membranes suspended in culture media. This format also facilitates compartmentalisation of the culture (apical-basal compartments), essential for developing epithelial and endothelial tissues in vitro,^{1,2} as well as various types of co-cultures (e.g., cancer-immune cell interaction,³ host-microbe interactions⁴). Assessment of the barrier function or integrity of such co-cultures and tissues is typically done via permeability assays using tracer molecules. For real time evaluation, electrical measurement of Trans-Epi/Endothelial Electrical Resistance (TEER) is an attractive label-free alternative approach.^{5,6} In contrast, electrical monitoring provides a rapid and non-destructive assessment of cell health and state, following the evolution of the tissue growth and function in real time.⁷⁻⁹ The most common techniques for measuring TEER involve either single-frequency measurements using ‘chopstick electrodes’ (e.g., EVOM),^{10,11} or electrical impedance spectroscopy (EIS), which offers a more detailed characterisation of cellular health and status, due to current measurements in a broad frequency spectrum (e.g., CellZScope, Nanoanalytics GmBH,¹² or other various electrodes^{13,14}).

In general, impedance-based cell monitoring is a highly sensitive method for monitoring cell growth, proliferation and differentiation.¹⁵⁻¹⁷ However, the sensitivity of this method is highly dependent on whether the cells are directly in contact with the electrode; electrodes where cells are directly adhered on the surface provide highly sensitive recordings of cell function. However, this planar culture configuration impedes transport of nutrients and other molecules (e.g., potential drug compounds) across the layers, thus prohibiting studies looking at such mechanisms. ¹⁵⁻¹⁷ In addition, TEER cannot be accurately measured on cells attached to an electrode, as the 2D environment and, mainly, the absence of the apical/basal compartments, do not provide cells with the necessary cues to differentiate towards the desired tissue phenotype: polarised, intact and selectively permeable epi-/endo-thelial barriers. In the case of cells grown on Transwell® inserts, TEER measurements are taken of the cell layer suspended between electrodes. The distance from the electrodes however, means that TEER measurements are typically only possible with cell layers that are characterised by high levels of resistance, typically >20 Ohm-cm². The constraints of the electrical circuitry also require a continuous cell layer, without holes or defects, for successful monitoring.

Recent trends in cell biology have emphasised a move away from growing cells in monolayers, towards formats that better recapitulate the native tissue environment, allowing for co-cultivation of cells and development of stratified tissues.^{18,19} The inadequacy of 2D cell cultures to generate the required information for drug efficacy and toxicity has been a major cause of high attenuation rates in the drug discovery process.^{20,21} Importantly, the shift from 2D to 3D has mostly been thought of in terms of cells grown on solid supports and has not yet encompassed the need to assay different biological compartments, as exemplified by cells grown in Transwell culture setups. To date, the complexity of biological tissues on Transwell® inserts has been limited to growing tissue-representative cells on either side of the stiff, plastic Transwell membrane and, possibly, cultivating another cell type on the bottom of the well plate, supported if necessary by conditioned cell growth medium in the relevant compartments.²²⁻²⁴ More advanced models, such as spheroids and organoids, better mimic the tissue 3D architecture and functionality,²⁵ but drug development and screening studies with such biological systems suffer because access to the luminal/apical side is possible only via microinjection.²⁶ Tissue-engineered constructs (i.e., scaffolds, hydrogels, fibrous meshes) provide a means for spatial cell organization in 3D on supports that mimic stiffness of tissues, predominantly based on natural (i.e., collagen, chitosan) or synthetic (i.e., poly(vinyl alcohol), poly(lactic acid)) polymers.^{19,27} Although these scaffolds capture the 3D architecture,¹⁹ evaluation of living tissues can be challenging due to difficulties in imaging through 3D structures. Such limitations can be overcome by using other means of characterization, for example electrical measurements, provided that the scaffolds are electrically active.^{18,19}

It has previously shown that conducting polymer (CP) scaffolds can be used as substrates for 3D cell cultures, as well as active elements in electronic transducers for label-free and real-time cell sensing.²⁸⁻³³ The porous nature of the CP scaffolds, allowing for flow of nutrients/compounds into and out of the scaffold, creates a highly favourable environment for cell growth. As cells attach, proliferate and differentiate within this porous network, they are monitored by the CP electrode, the electrical properties of which change due to the activities of these cells. Through this dynamic interfacing of cells with the CP electrode, the necessary intimate cell-electrode coupling is established, allowing for highly sensitive and accurate cell sensing. Thus, real-time information acquisition on different stages of cell growth, and differentiation of barrier forming cells, as well as other non-barrier (i.e. low resistance) cell types, is possible. However, these devices suffer from lack of throughput, which is problematic for routine biological assays.²⁸⁻³³

Demonstrated herein is a bioelectronic transmembrane (e-transmembrane; porous scaffold membrane) device compatible with conventional tissue culture well-plate configurations, and capable of hosting and monitoring biologically complex, 3D cell cultures, introducing a considerable increase in throughput. Specifically, PEDOT:PSS based scaffolds were engineered to function both as separator membranes for compartmentalized cell cultures, and as electronic elements for recording cell growth and crucial tissue parameters. Adopting an electrode configuration setup, the 3D e-transmembrane device can electrochemically monitor changes associated with the presence of any commonly used adherent cell types, both in the bulk of the scaffold and at the outer interfaces. To validate the compatibility and functionality of the system, the e-transmembrane was cultured in situ with human organ-specific cells, modelling the intestinal epithelium or the vascular endothelium. EIS measurements, carried out throughout the culturing period, allowed determination of signatures from different cell types, assessing cell growth and extracting barrier function parameters (i.e., TEER). Taken altogether, compatibility with well-plate formats, the 3D biological architecture, the apical-basal compartmentalisation and the dynamic cell monitoring capabilities render the e-transmembrane platform a universal tool for biologists, laying foundation for the next generation high-throughput drug screening assays.

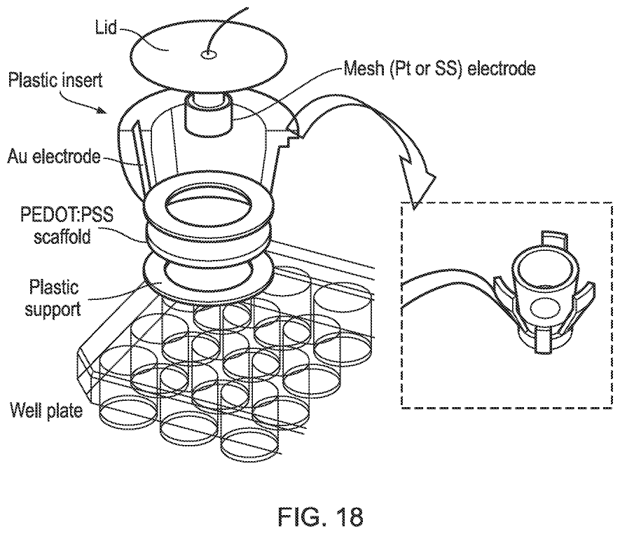

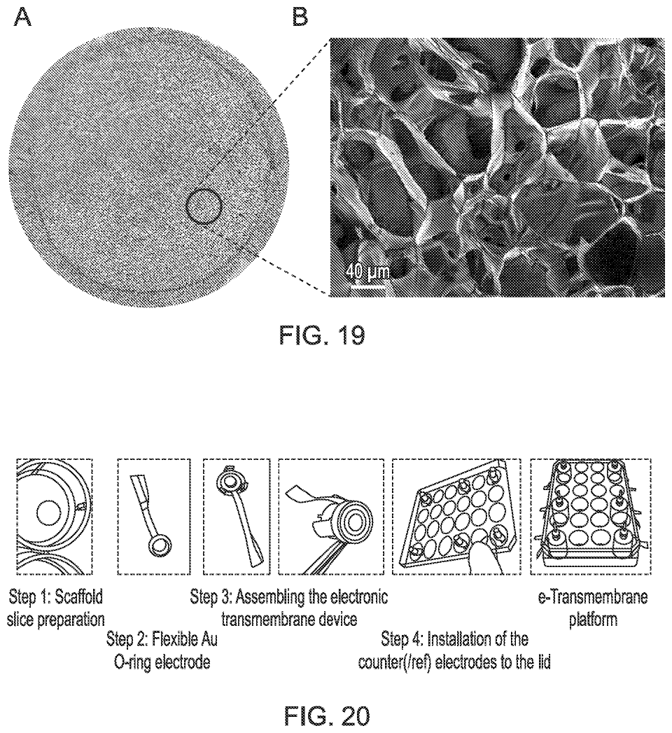

The individual components of the e-transmembrane platform, including a well-plate-compatible insert to accommodate the contact electrode(s), the plastic supports and the scaffold transmembrane, are shown in FIG. 18. High surface area mesh electrodes were installed in the lid, acting as counter electrodes for the electrochemical measurements. The transmembrane, separating the basal and the apical compartment, consists of a 3D CP membrane based on slices of a PEDOT:PSS scaffold, prepared using a micro-vibratome. By tuning the slicing conditions and the fabrication parameters we created conducting scaffold slices as thin as 100 μm (FIG. 19A), without affecting their structural integrity and pore uniformity. The resulting structures displayed a macroporous morphology and a network of interconnected pores with a high level of porosity, as shown in the SEM image in FIG. 19B. It should be noted that, although pores often appeared to be aligned in the longitudinal direction, no specific pore orientation was observed. The average pore size, determined through image analysis of SEM scans, was found to be 57.5 (±7.4) μm, while the estimated porosity was 78%, which is consistent with microCT measurements on scaffolds of the same composition.³⁴ Comparing these values with previous reports, it was assumed that the morphological and dimensional characteristics of the transmembrane constructs were adequate for efficient cell infiltration, proliferation and survival throughout the 3D structure. Intrinsic limitations arising from the 3D dimensionality of the porous structures (i.e., inhomogeneous cell distribution, variable pore size, cell culture medium access) were addressed by tailoring the thickness of the scaffold transmembrane in a controlled manner. Unlike previous studies on PEDOT:PSS scaffolds, which were based on bulky mm-thick constructs and thus heavily relied on perfusion capabilities, the thin transmembrane architecture can ultimately improve the accessibility to the surrounding media and allow for sufficient mass transport of nutrients and oxygen through the pores, even under static conditions.

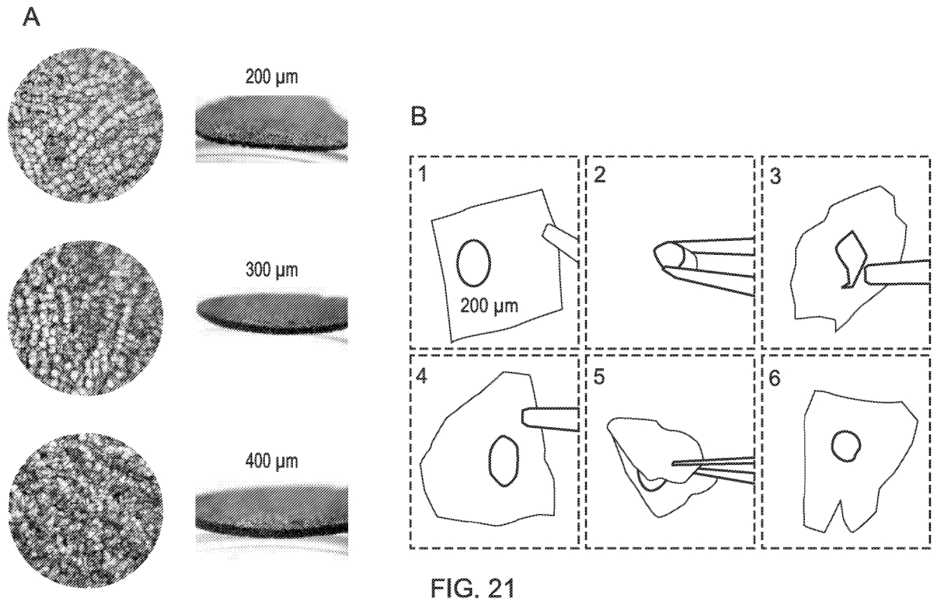

The different steps for the fabrication of the device components and the platform assembly are shown in FIG. 20. The first step involves the lyophilization of the PEDOT:PSS solution and the sectioning of the resulting scaffolds. Typically, the thickness of the scaffolds slices ranges between 100 and 500 μm, with the thickness of 400 μm being the selected condition for the provided cell studies, unless otherwise stated (FIG. 21A).

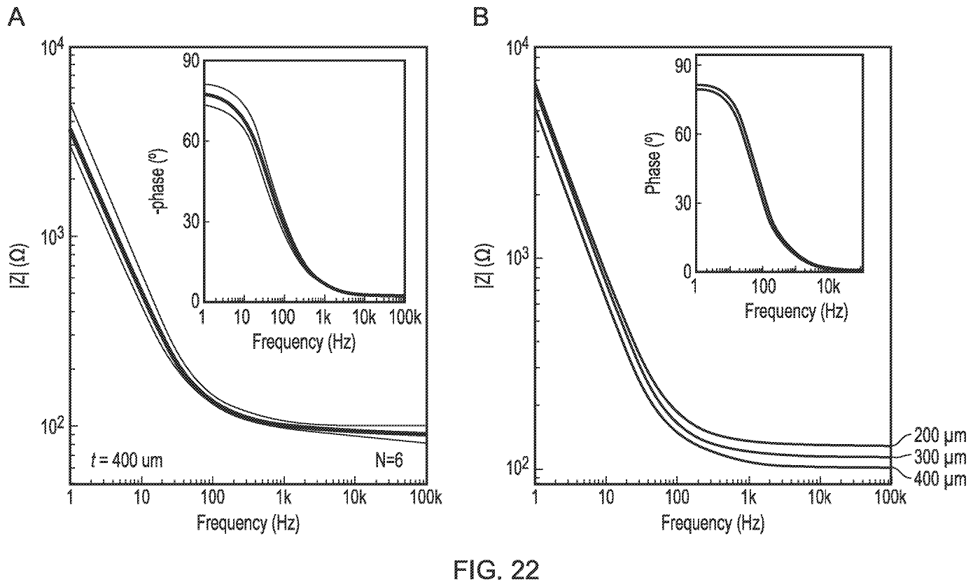

Characteristics such as pore size, porosity and morphology can be tailored on demand by changing the process parameters and/or the solution composition (See FIGS. 3 to 5). For example, the use of cryoprotectants (i.e., dimethyl sulfoxide, methanol) can substantially decrease the average pore size, while mixing PEDOT:PSS with other polymers (i.e., PVA) or biomolecules (i.e. polysaccharides) can alter the pore morphology and the surface properties of the scaffolds. Gold was evaporated onto polyimide substrates (Kapton®) to create flexible, O-ring electrodes, which were inserted into the plastic insert to ensure direct contact with the PEDOT:PSS scaffold slices. Additionally, an insulating layer of PDMS (or Parylene-C) was layered onto the gold surface, covering the part, in direct contact with the electrolyte to avoid interference in electrochemical measurements. Finally, the platform was completed by integrating counter electrodes on the well plate lid, allowing for in situ electrical monitoring, avoiding immersing external circuitry or operating the system in a biosafety cabinet. Handling of ultrathin porous scaffolds can be challenging, and even mild external stresses can affect their structural integrity. In an attempt to study the durability of the PEDOT:PSS transmembrane (porous scaffold membranes) a crumpling test was performed, where a 200 μm-scaffold slice is transferred onto a flexible polymeric substrate and randomly folded several times as shown in FIG. 21B. Interestingly, no major defects are observed after un-crumpling and transferring the slice back to the liquid. The electrochemical performance of the transmembrane electrodes is assessed by means of EIS. As shown in the Bode plots of FIG. 22A, the electrodes exhibit an ohmic behaviour in the high-to-mid (100 kHz-0.5 kHz) frequency region (as indicated by the line parallel to x-axis) and a capacitance-dominated regime at the low-frequency range (<100 Hz). This is also identified in the phase plot (see inset), where a shift between 0° to nearly 90° can be observed. As expected, the overall impedance magnitude scales down with the thickness increase, due to the larger effective surface area (FIG. 21).

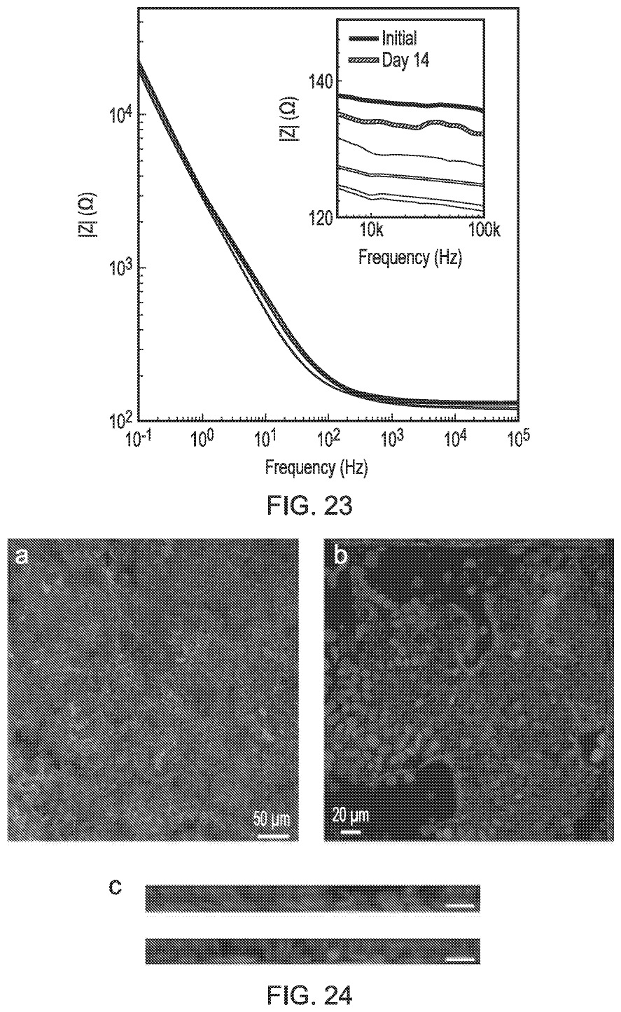

Nevertheless, the various transmembrane electrodes exhibit low impedance at 1 kHZ, with values ranging between 108Ω and 135Ω. It is noted that the e-transmembrane device displays remarkable stability after 14 days in cell culture medium corroborated by the almost unchanged EIS spectra in FIG. 23.

The feasibility of the proposed e-transmembrane platform and its standing with respect to the current Transwell-based assays were evaluated using both an epithelial and an endothelial cell model. As seen in FIG. 14 the 3D transmembrane comprises of a confluent human epi- or endo-/thelial cell monolayer and human telomerase immortalized fibroblasts (TIF LifeAct) grown in the bulk of the scaffold. It has been recently proposed that a strategy for engineering complex, stratified tissues in 3D electroactive scaffolds, in which the first step involves cultivation of electroactive substrates with fibroblasts, serving as a guide for tissue organisation upon subsequent integration of other cell types, according to the type of the tissue model under development.³⁴ Here, this strategy was adopted to form a baseline for the 3D e-transmembrane models to test if this novel 3D cell culture platform can support concurrent development/maintenance and real-time monitoring of human epithelial and endothelial barriers.

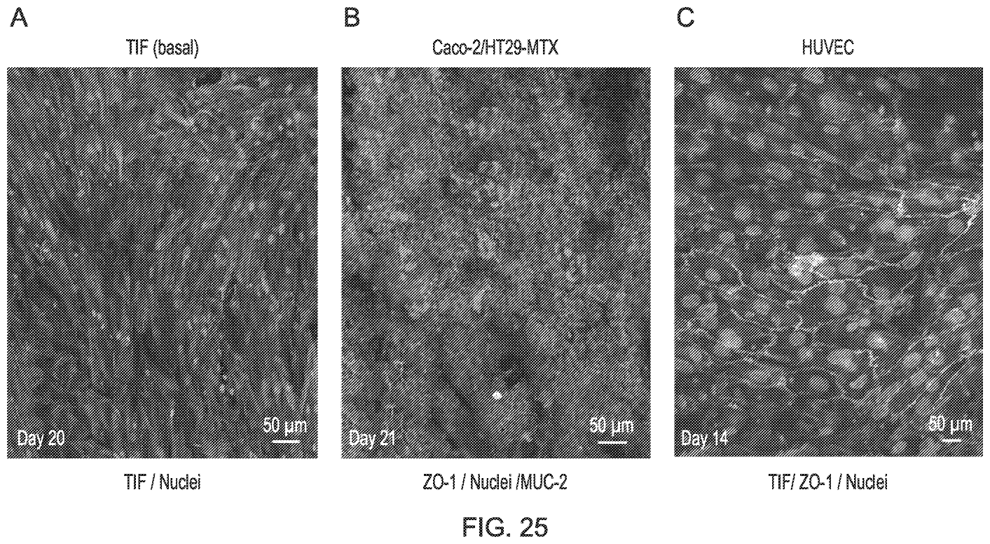

For the 3D epithelial model, a co-culture of intestinal epithelial cells, comprising Caco-2 cells and HT29-MTX cells (IECs) in a 3:1 ratio was used to reflect the in vivo conditions, as described.³⁴ These cells were seeded on the apical surface of the 3D transmembrane, pre-cultivated with fibroblasts for four days (on day 5 of the culture), following the same ex situ process; the 3D transmembranes of each platform are transferred in dedicated wells of a non-treated 12-well-plate and the cell suspension (either TIFs or IECs) is added on the apical surface of each scaffold. Following a two-hour incubation period, the 3D transmembranes are then transferred back to the e-transmembrane platform (treated/cell culture grade 24-well plate), complete growth medium is added in both apical and basal compartments and the resulting 3D tri-cultures are routinely maintained for another 3 weeks. As expected, confocal microscopy at the end of the ˜1 month-long experiment, revealed a continuous monolayer of polarised intestinal cells (see FIG. 24), anchored on top of the pre-established extensive TIF-derived fibrillar network, intercalating the porous network of the electroactive substrate (FIG. 25A). Labelling for typical intestinal biomarkers revealed the differentiation of the intestinal layer towards a tight barrier tissue, exhibiting typical apical brush border characteristics, including consistent expression of tight junctions (i.e., zonula occludens-1 (ZO-1)), and mucin proteins (i.e., mucin 2 (MUC2)), (FIG. 25B).

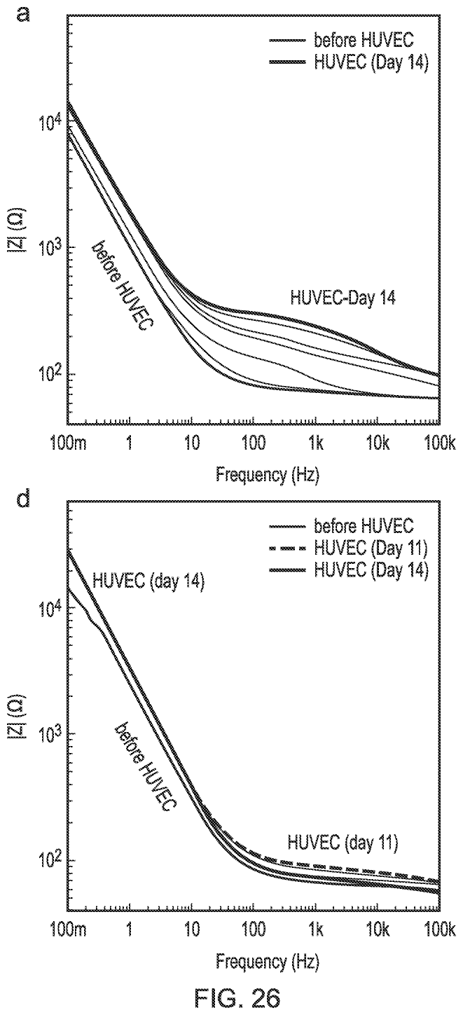

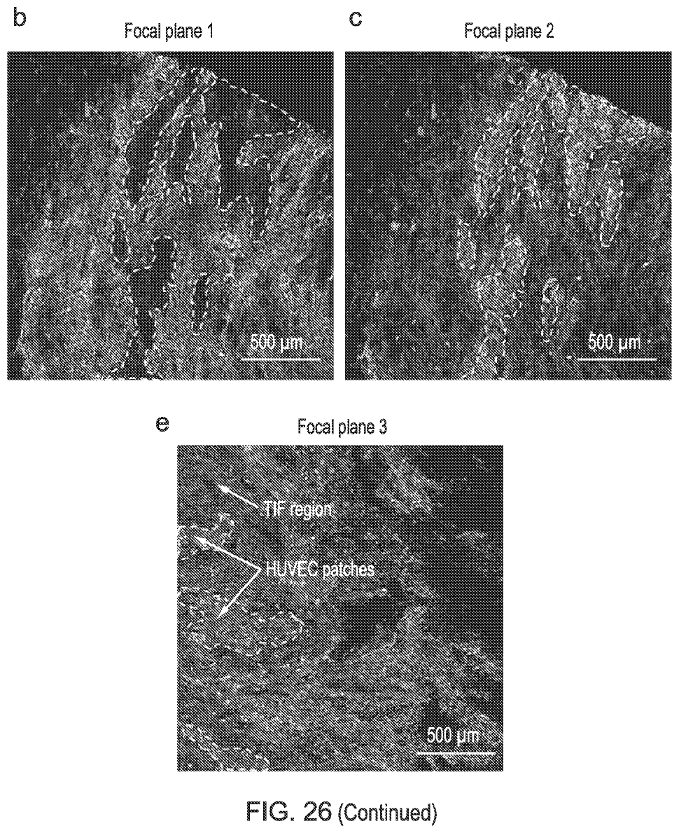

Similarly, human umbilical vein endothelial cells (HUVECs) were used for the 3D endothelial vascular model. To create this model, scaffolds were initially seeded with fibroblasts to provide a supporting layer for generating the vascular membrane. After 4 days, HUVECs were added onto the transmembrane devices. The devices were cultured under dynamic fluid conditions to promote the genesis of vascular junctions, as fluid shear has been hypothesized to improve vascular phenotypes.³⁵⁻³⁸ The resultant cellular structuring showed consistent coverage of endothelial cells across the entire 6 mm diameter surface of the scaffolds with consistent adherent junctions, as demonstrated with vascular endothelial (VE)-cadherin staining (FIG. 25C). The surface exhibited some roughness, which was confirmed to be covered by endothelial cells through multi-level imaging on a confocal microscope (FIGS. 26C and D). The devices were cultured for a total of 14 days, including the initial mono-culture of the fibroblasts.

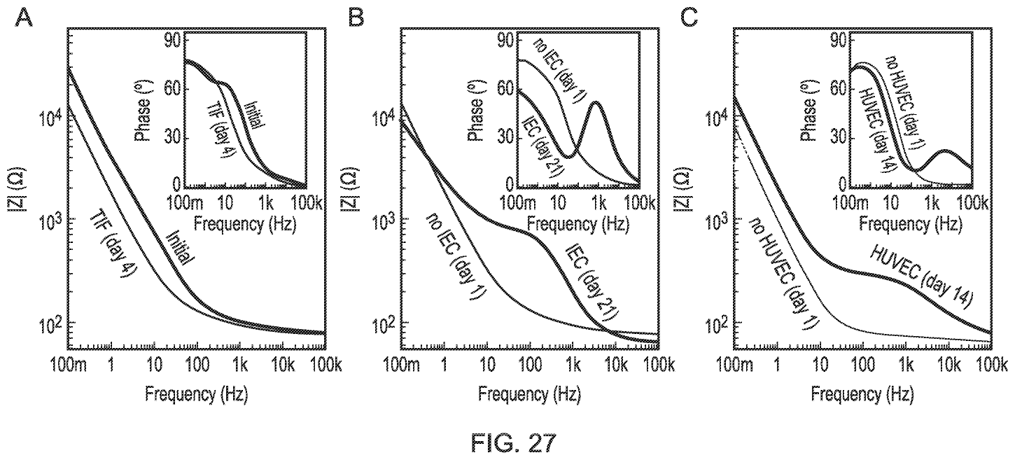

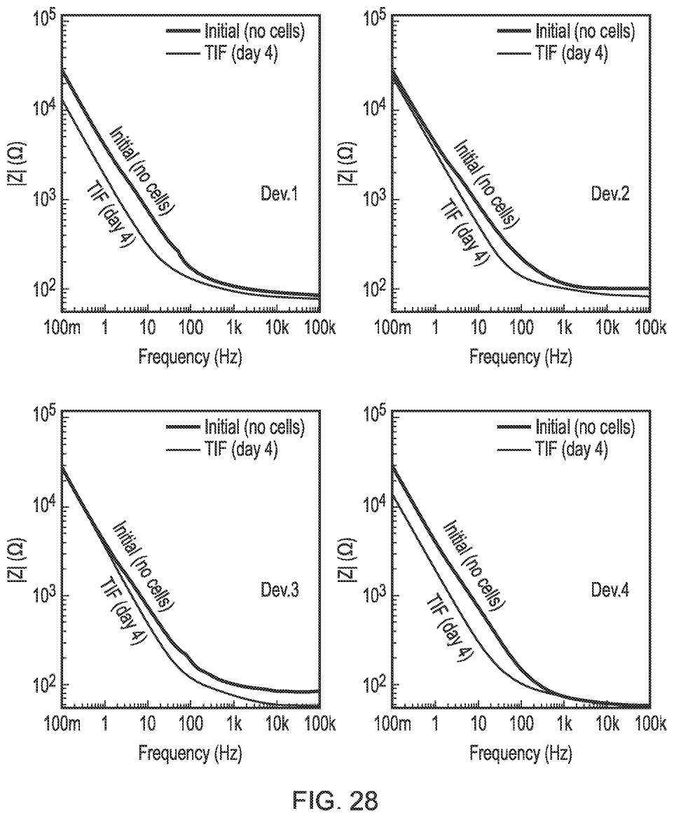

The electrical properties of the 3D cell cultures inside the e-transmembrane platform were evaluated using EIS similarly to previous studies.^{28,29} As shown in the EIS Bode plot of FIG. 27A, the incorporation of TIFs decreases the impedance predominantly in the capacitive region, in line with previous observations, consistent with an increase in the electrode capacitance due to the presence of the cells and the subsequent secreted ECM. As seen in FIG. 28 the effect of fibroblasts on the 3D scaffolds fluctuates in terms of magnitude and in some cases an additional shift in the ohmic region can be also observed. It is believed this is an indication of the degree of infiltration of the TIFs into the scaffolds as cells accumulating in the top interface (electrode/electrolyte) would result in a change in the ohmic resistance whereas cells that have infiltrated the scaffold would predominantly affect the capacitive region. The fibroblast-cultured scaffold (monitored after 4 days of culture) represents the baseline of the subsequent measurements in both epi- and endothelial cultures.

The presence of a confluent epithelial layer (after 25 days in total—21 days of IEC culture) results in an increase in the magnitude of the complex impedance in the mid-high frequency range, corresponding to a polarised, fully differentiated epithelial barrier lined with mucin (as cross-validated optically). Similarly, the Bode phase plot indicates the formation of a time constant in the ˜1 kHz frequency which corresponds to the paracellular resistance. The observed impedance profile is in agreement with literature when using cell monolayers atop planar CP electrodes.

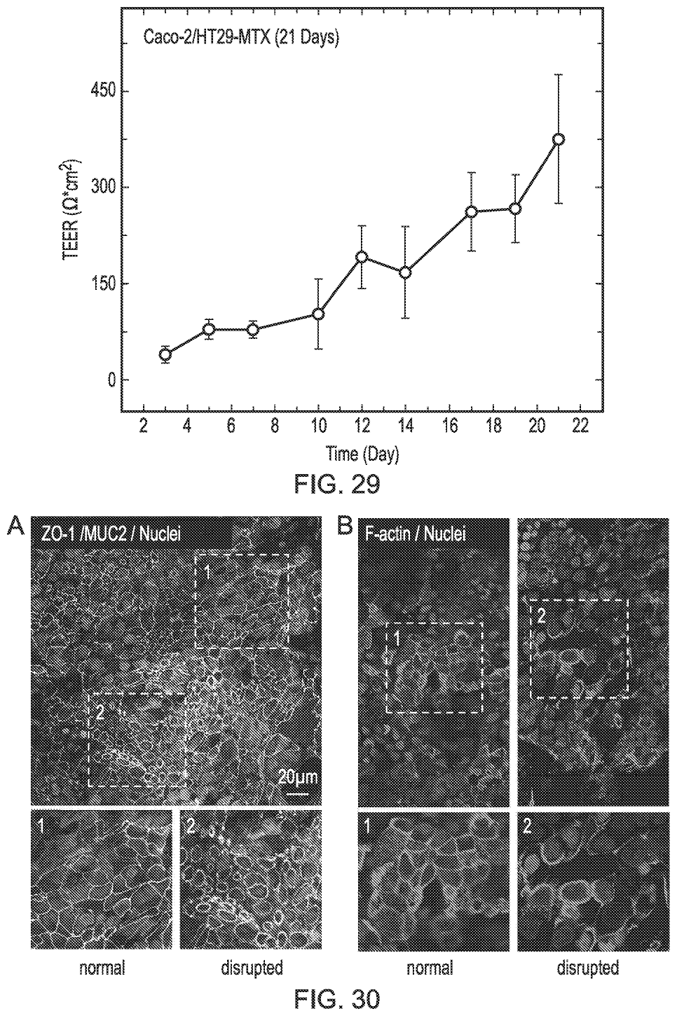

Similar to the epithelial model, the confluent endothelial layer resulted in analogous changes in the impedance spectra, albeit less pronounced, given the lower TEER values that have been recorded for HUVECs versus epithelial cells. While many studies have monitored TEER for the endothelial cells of the blood brain barrier, few have examined TEER for other vascular applications. In this context, measured TEER values for HUVEC cultures typically range from −10-20Ω·cm² or higher in some cases. A time constant is also noted on the Bode phase plot at slightly higher frequencies compared with IEC cultures. This result, as well as the decreased impedance, are consistent with a reduced TEER value for the HUVEC populations compared with measurements on epithelial cells. Following, it was demonstrated that the real-time dynamic monitoring of IECs growth by extracting the TEER parameter by modelling the impedance spectra using the equivalent circuit shown in FIG. 15 The equivalent circuit used herein comprises three elements, the cell layer expressed as a Rₘ//Cₘ, the 3D electrode expressed as a constant phase element (CPE) accounting for a non-ideal capacitor (typically representing CP scaffolds or electrodes ref) and the electrolyte (including any other series resistors) expressed as Rₛ. It is noted that the Rₘ accounts for the TEER of the cell layer. FIG. 29 shows the evolution of the TEER for the 3D epithelial model over time. As expected, during the whole duration of the cell culture, TEER increases reaching a plateau after ˜17 days of culture, corresponding to the tissue development stages of cell polarisation, tight junction formation and sealing of the paracellular pathway towards an intact barrier.

Then, the ability of the e-transmembranes to detect real-time breaches in the intestinal barrier was tested. In native epithelial tissues, such as the intestinal epithelium, lateral junctions are formed of complex networks of various types of intercellular junctions (e.g., tight junctions and adherens junctions) to form physical barriers. Analysis of the integrity of the junctional network can be indicative of the barrier tissue state, a property that is critical in certain pathophysiological conditions, such as inflammation.^{39,40} The dependence of tight and adherens junctions on Ca²⁺ is well-established in literature,⁴¹ where depletion of Ca²⁺ results in disruption of the physiological dynamics of the junctional network (i.e., endocytosis of junctional proteins), disaggregation of adherent cells and breakage of the barrier integrity, which is reflected in the increased permeability and lower TEER values of the epithelia.^{42,43} Ca²⁺ chelation, however, allows for the epithelium to recover its integrity via re-distribution of the proteins in their physiological functional and structural organisation, resealing the intercellular junctional network.^{42,44,45} The calcium-switch assay is commonly used to characterise the barrier integrity of epithelia in vitro by exposing the barrier to calcium chelators, such as EGTA (Ethylene glycol-bis(beta-aminoethyl ether)-N,N,N′,N′tetra acetic acid), thus mimicking the disruption of the junctional complexes and opening of the intercellular space under pathological conditions (e.g., inflammations, pathogen invasion, drug toxicity).^{43,46-48}

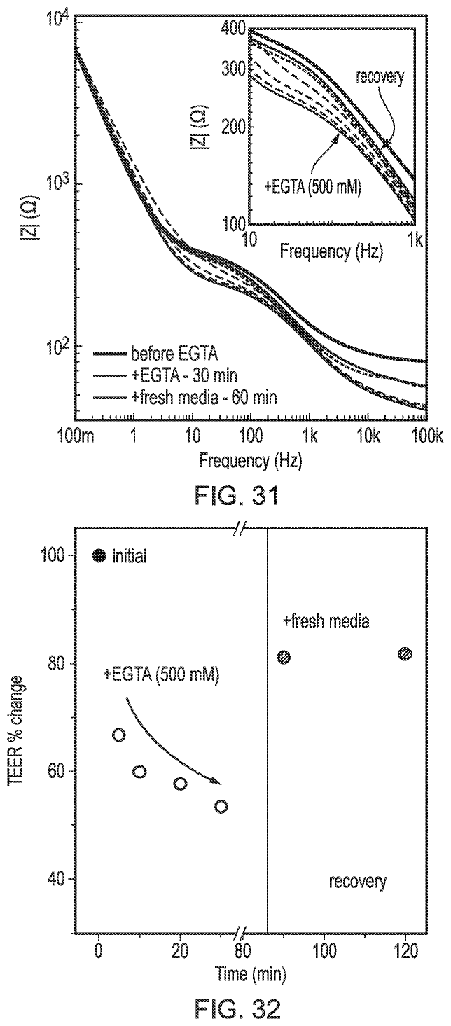

The apical compartment of the 3D epithelial transmembranes was exposed to 500 mM of EGTA and its effects on the barrier was electrically monitored for an hour, after which the cells were thoroughly washed with fresh, Ca²⁺-containing, complete cell growth media. The recovery of the barrier was also monitored electrically, while at the end of the experiment confocal microscopy was employed for cross-validation of the findings, investigating the effects of EGTA on ZO-1 junctional proteins. FIG. 30A illustrates a section of the apical domain of the 3D epithelial transmembrane model, at the end of the experiment, revealing the partial recovery of the barrier, with regions of the junctional network exhibiting the physiological morphology (FIG. 30A1) and regions where adjacent cells are detached and ZO-1 is internalised and co-localised with individual cell periphery (FIG. 30A2). In addition, the cell cytoskeleton was visualized by staining for filamentous actin, found in abundance in the microvilli structures of the apical brush border of intestinal cells. Similar to the tight junction imaging, two distinct areas in the immunostained sections of the barrier were observed (FIG. 30B); one area exhibits the expected mosaic-like pattern, with consistent microvilli formation on the apical surface of cells (red dots in the body of cells are the top tip of microvilli; FIG. 30B1) and the other area reveals cell detachment from the layer and perijunctional actin filament disassembly FIG. 30B2. This was expected, as Ca²⁺ is known to be critical for filamentous actin organisation in intestinal cells,⁴⁹ associated with the assembly and stability of perijunctional cytoskeleton and, in turn, with the integrity and turnover of epithelial junctions.⁵⁰ Finally, it is worth noting that the mucin domains previously observed (see FIG. 25C) were not detected here. This can be also attributed to EGTA, which is known to interact with mucin gels, affecting their viscocity and inducing rapid swelling, hydration and eventually dispersion of mucins.⁵¹ Overall, these findings suggest that the 3D epithelial barrier only partially recovered its integrity upon reintroduction of Ca²⁺ in the extracellular environment. The relatively short recovery period, combined with the concentration of EGTA used, could explain this partial/incomplete resealing of the barrier, however, the key point of this imaging assay is that it cross-validates the trend observed in the complex impedance spectrum and TEER values (see FIGS. 31 and 32).

### Methods

### Preparation of Devices for Cell Seeding:

After the e-transmembrane platform is fully assembled, it undergoes sterilisation. Each device is placed in dedicated wells of a sterile 24-well plate, pre-filled with in 70% ethanol, while ethanol is also added in the apical compartment so the lid electrodes can be fully immersed in it. The lids are thoroughly sprayed and wiped with ethanol too. Sterilisation is followed by careful and thorough rinsing with sterile water and PBS and then the transmembrane scaffolds are immersed in complete growth media of TIF cells (see below) for 2 hrs, to allow for protein adhesion on the scaffold surface. For this step, each e-transmembrane is removed from the well, the bottom of which is then filled with media. Then each device is carefully moved back to the well, gently pushed in with a pair of tweezers in order to remove any air bubbles that could be trapped in the basal compartment. Media is also added in the apical compartment of the devices, making sure that the reference/counter electrodes can be fully immersed in each well, to facilitate electrical monitoring (at this stage first measurements are taken). Prior to seeding, scaffolds are rinsed with fresh medium and, with a pair of tweezers, they are placed in the middle of the dedicated well in a non-treated 12-well plate.

### Epithelial Cell Model:

The generation of the 3D intestinal epithelial barrier on the e-transmembranes was based on the biological model and the two-step seeding strategy, originally described in “Pitsalidis, Charalampos, et al. “Transistor in a tube: A route to three-dimensional bioelectronics.” Science advances 4.10 (2018): eaat4253.”, adapted to the devices used here. 400 μm thick PEDOT:PSS scaffolds were used for all the experiments. Also see Moysidou, Chrysanthi-Maria, et al. “3D bioelectronic model of the human intestine.” Advanced Biology 5.2 (2021): 2000306.

First, a supporting lamina-propria layer was established in the bulk of scaffolds by culturing Telomerase Immortalised Fibroblasts (TIFs) labelled with Red Fluorescent Protein (RFP; TIF LifeAct) (a gift from Ellen Van Obberghen-Schilling, Institut de Biologie de Valrose). TIF LifeAct (P9-12) were routinely maintained in Advanced DMEM (Gibco) supplemented with 20% FBS (Sigma Aldrich), 1% Glutamine (Gibco), 2% HEPES (Gibco), 0.5% penicillin-streptomycin (10,000 U/ml, Gibco) and 0.1% Gentamycin (Sigma Aldrich), harvested with 0.25% trypsin. 250,000 fibroblasts, suspended in 70 μl of media, were ‘dropped’ on the top surface of each scaffold disc with a pipette. Cells were then incubated (within the non-treated well-plate) for 2 hours to allow for cell attachment and adhesion. After 2 hrs, each device was carefully moved back to its dedicated well of a sterile (treated/cell culture grade) 24-well plate, prefilled with fresh cell growth media. Again, with a pair of tweezers each device was gently squeezed in the well, displacing the air out whilst pushing in, in order to avoid formation and trapping of bubbles in the basal compartment. Fresh cell growth media was also added in the apical compartment, making sure that the counter/reference electrodes of the lid would be fully immersed in each device. These TIF 3D cultures were maintained for 4 days to allow cells to infiltrate the scaffolds and secrete ECM proteins. Medium was changed once.

On day 5 of TIF culture, the intestinal epithelial cells were seeded in the devices to generate the epithelial barrier model. To this end, the 3:1 Caco-2 and HT29-MTX co-culture described here was used (referred to as Intestinal Epithelial Cell (IEC) co-culture). Both Caco-2 and HT29-MTX cells were purchased from ECACC and were routinely maintained before all experiments. Caco-2 cells (P50-58) were cultured in Advanced DMEM (Gibco), supplemented with 10% Foetal Bovine Serum (FBS, Sigma Aldrich), 1% M Glutamine (Glutamax-1; Invitrogen), 1% penicillin-streptomycin (10,000 U/ml, Gibco) and 0.1% Gentamicin (Sigma Aldrich). HT29-MTX-E12 (P54-59) cells were maintained in Advanced DMEM (Gibco), supplemented with 10% FBS, (Sigma Aldrich) and 1% penicillin-streptomycin (10,000 U/ml, Gibco). Prior to seeding cells were harvested with 0.5% and 0.25% trypsin and mixed to a single cell suspension in the desired ratio. Each pre-seeded with TIFs device was removed from the 24-well plate, after removing the cell medium from both compartments, and placed in the centre of a dedicated well in a sterile, non-treated 12-well-plate. Again, 250,000 IEC cells (3:1 Caco-2:HT29-MTX), suspended in 70 μl of media, were ‘dropped’ on top of each scaffold with a pipette and cells were incubated for 2 hours to allow for cell attachment and adhesion, before moving them back in the e-transmembrane platform—with the same method as described before—filled with fresh, co-culture medium, the composition of which was tweaked as described here. This tri-culture system was maintained for another 3 weeks (total culture period 26-27 days), with media being changed every other day, before samples were fixed with 4% paraformaldehyde (PFA, ThermoFisher) and stored in PBS for optical characterization.

### Calcium Switch Assay:

To investigate the potential of the 3D transmembranes to detect disruption of the epithelial barrier in real time, a calcium switch assay was used. More specifically, we added in the apical compartment of the gut epithelim 3D transmembranes 500 mM of Ethylene Glycol Tetraacetic Acid, diluted from stock in cell growth medium (Alfa Aesar EGTA 0.5 M aqueous solution, J60767) and monitored its effects for 60 mins. Cells were then washed 3× with fresh complete growth medium, containing Ca²⁺. The recovery of the epithelial barrier was also monitored electrically for the following two hours, before cells were fixed with 4% paraformaldehyde, washed with PBS and stored for imaging.

### Epithelial Model—Optical Characterisation:

All samples were fixed in 4% paraformaldehyde (PFA, ThermoFisher) for 20 mins, at room temperature. After thorough washing with PBS, each scaffold was stored at 4° C. until ready to use for optical analysis. Prior to immunostaining, cells were permeabilised in 0.1% v/v Triton X-100 (Fisher) for 15 mins and then blocked for nonspecific binding with 1% wt/v BSA (Fisher) and 0.1% v/v Tween-20 (Fisher) in PBS for 1 hr at room temperature. To label the samples for actin filaments, tight junction protein zonula occludens-1 (ZO-1), mucin protein 2 (MUC2) and nuclei, the following primary and secondary antibodies were used: Rb polyclonal anti-ZO-1 (ThermoFisher Scientific) Ms monoclonal anti-MUC2 (abcam), Bisbenzamide H (Hoechst 33342, abcam), Goat-anti-rabbit Alexa Fluor 488 (abcam), Goat-anti-mouse Alexa Fluor 647 (abcam), Phalloidin-iFluor 594 Reagent (abcam). Slices were then placed on microscopy plates and were maintained hydrated with PBS during imaging with the epifluorescence/confocal microscope Axio Observer Z1 LSM 800 (ZEISS).

### Endothelial Cell Seeding:

TIFs were seeded onto 500 μm thick PEDOT:PSS scaffolds. Scaffolds were suspended in 1 mL of TIF media (see below) and placed onto an orbital shaker (IKA KS 260 Basic, IKA; Staufen, Germany) rotating at 60 rpm. 200,000 TIFs in 75 μL of media were pipetted into the well containing the scaffold in the device and left on the orbital shaker. After 24 hrs, the media was removed and replaced with 2 mL of fresh TIF media. The devices were cultured under static conditions for the following 3 days. HUVECs (Lonza, Biosciences; Basel, Switzerland) were added into the system in the same manner, applying 50,000 HUVECs within 75 μL of HUVEC media (EGM-2, Lonza Biosciences; Basel, Switzerland). After 24 hrs, the media was removed and replaced with fresh HUVEC media. These samples were left to culture statically for 2 days, at which point the orbital shaker was reinitiated to simulate blood flow. Cultures were carried out for an additional 7 days before samples were fixed for imaging.

### Immunofluorescence:

Samples were permeabilized with 0.1% triton (Fisher) in PBS for 10 min, washed 3 times with PBS, incubated in 1% bovine serum albumin (BSA) (Fisher) and 0.05% tween (Fisher) in PBS for 1 h, and washed 3× in PBS before antibody steps. Following these steps, samples were bathed in 1% BSA in PBS with a rabbit monoclonal VE-cadherin antibody (D87F2, Cell Signaling Technology; Danvers, MA, USA) for 1 hr and washed 3 times in PBS. Following primary antibody incubation, samples were bathed in 1% BSA in PBS with a goat anti-rabbit IgG AlexaFluor 488 secondary antibody (ab15007, Abcam; Cambridge, UK) for 1 hr and washed 3 times in PBS. Samples were stained with Hoechst 33342 ( ) for 15 min and washed 3 times in PBS.

### Confocal Imaging:

Samples were imaged on a Zeiss LSM 800 (Zeiss; Oberkochen, Germany). Images were collected using a 561 nm laser to image RFP fluorescence, a 488 nm laser to image Alexa Fluor 488 fluorescence, and a 405 nm laser to image Hoechst 33342 fluorescence. White light images were collected using an electronically switchable illumination and detection (ESID) module, fitted in the Zeiss LSM 800.

### Example 3—Developments of Device

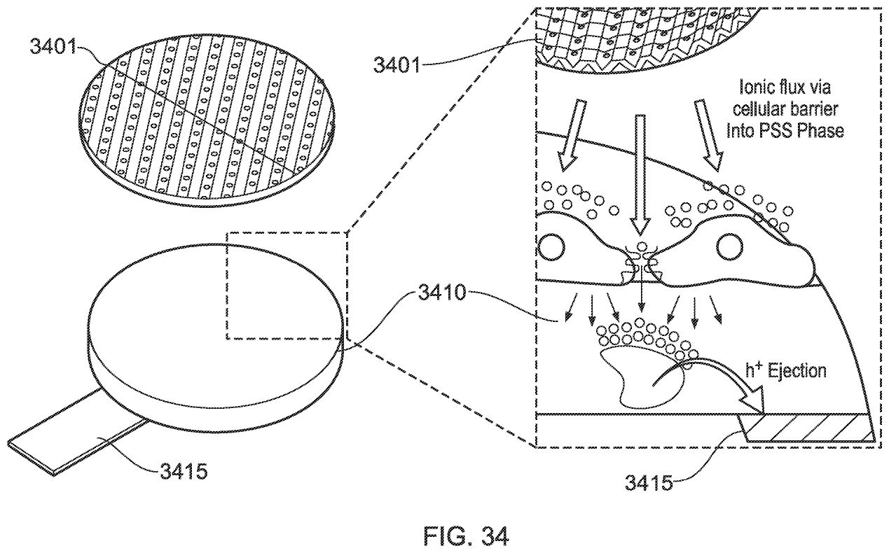

The standard configuration of the Transmembrane (TxMem) device is illustrated in FIG. 34 and consists of a Platinum mesh electrode (3401) apically and a PEDOT:PSS scaffold (3410) basally, which is in contact with a flexible gold substrate (3415). As per electrochemistry convention, the apical electrode is referred to as the counter electrode and the PEDOT:PSS/Au system is referred to as the working electrode.

The mechanism of operation of the device can be summarized as follows:
A potential difference is applied between the counter and working electrodes, which instantiates an electric field between the two.
Ionic/charged species present in the electrolyte bulk between the two electrodes, immersed in the field, experience a force applied nominally along the vertical axis (perpendicular to the primary electrode planes).
Ionic species infiltrate the PSS phase of the working electrode and conduct, aggregating at the PEDOT-PSS interface, thus forming Electric Double Layers (EDLs).
The increase in surface charge density on the surface of the PEDOT crystallites induces ‘hole’ (polaron) ejection into the Au phase, which manifests as a measurable electric current.
If a biological barrier is present on the surface of the scaffold, the flux of the ions is impeded, resulting in a decrease in the measured conductivity (increase in impedance).
As the system in highly nonlinear, the applied potential consists of a DC (constant) bias voltage and a small AC (sinusoidal, oscillating) stimulus voltage. The ratio of the AC Stimulus voltage and the AC component of the induced current yields the linearized transfer function, denoted as the impedance. (Technique referred to as Electrical Impedance Spectroscopy (EIS)).

While the TxMem is framed by conventional electrochemistry, its primary departure from that field is due to the fact that the system is generally non-Faradaic (without redox reactions) and unlike conventional electrochemical endeavour the objective of the measurement setup is not to measure the rate and nature of Faradaic processes occurring within the electrolyte bulk, or at the electrode-electrolyte interface of the working electrode. In general, the TxMem device measures transport phenomena as well as the interfacial impedance of the electrode-electrolyte interface of polarizable (i.e. non-Faradaic) electrodes.

The corollary of this statement is that, the design decisions made which are motivated by electrochemistry dogma are points for adaptation and potentially improvement. In particular, the apical electrode is made from Platinum as it is inert—a requirement for the measurement of Faradaic processes as it is certain that Pt will not participate in any reactions (thereby confounding the measurement). As the TxMem is not designed to measure Faradaic processes, the material restriction can be relaxed from inert, polarizable (i.e. Platinum) to polarizable only (i.e. not necessarily inert/noble). This is noteworthy as Platinum is costly and cannot be connected directly to non-noble metals due to its proclivity for initiating galvanic corrosion. Thus mechanical connections may need to be made to gold contacts, which increase the cost and suffer from stability issues.

In addition, the counter electrode may cause issues, with an impedance contribution which dominates the low-frequency regime and occludes information contained within. The conventional approach to circumventing this contribution is the use of reference electrodes in negative feedback. Conventional reference electrodes are designed so as to have a interfacial potential which is constant over a range of voltages and currents (termed non-polarizable or Faradaic electrodes). This allows the applied voltage to be varied so that the dependence of the redox reaction under study on the applied voltage may be studied. Quasi-reference electrodes (such as polarizable electrodes) are generally not used, as their interfacial potential is not constant and so contributes to the measurement as a confounder.

As true reference electrodes are generally not compatible with biological systems (due to their toxicity) it is common to not use reference electrodes in systems which probe biological systems over extended/continuous time periods. However, for the specific case of EIS measurement, where a single bias voltage is applied, and in systems where redox currents are not the measurement objective, the constant interfacial potential requirement for reference electrodes is relaxed. Thus quasi-reference electrodes may be used without significantly degrading measurement quality.

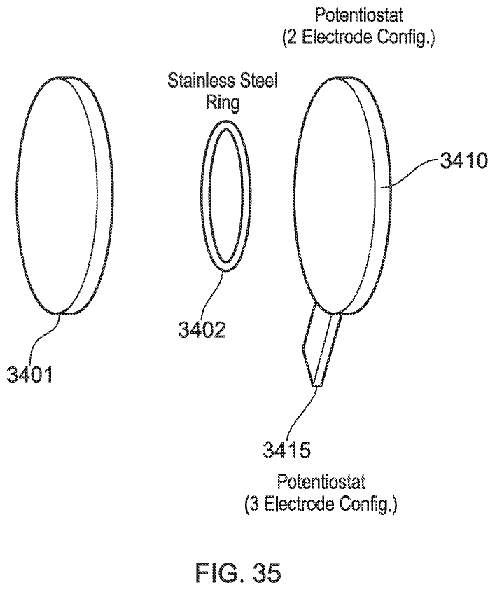

The apical and reference electrodes were altered/introduced with the design motivations as follows. As shown in FIG. 35, the device may be operated in a two electrode configuration (without reference electrode), where the addition of a reference electrode (3402) enables the use of the three electrode configuration.

Apical electrode (counter electrode)

As discussed above, the design criterion for the apical (counter) electrode is namely that it be constructed from a polarizable (i.e. non-Faradaic) material, where the greater the capacitance of the EDL (correlated positively with surface area for crystalline materials) the smaller the impact of the apical electrode on the measurement in the two electrode configuration (without reference electrode).

Stainless Steel was selected as a low cost material which is functionally polarizable at voltages compatible with cell viability (nominally |V|<1V). As before, a mesh is used to maximize surface area—the availability of the material allows for significantly larger pieces of mesh to be used in the construction. Furthermore, by using a phosphoric acid-based flux, stainless steel can be soldered to standard lead wire, which allows for a stable, low impedance connection.

### Reference Electrode

A quasi-reference electrode may be suitable for use in the TxMem system—this is non-obvious as, reference electrodes used to conduct EIS in order to measure biological systems are limited to standard reference electrodes, namely Silver/Silver-Chloride (which may or may not be separated from the electrolyte bulk by a hydrogel or salt bridge).

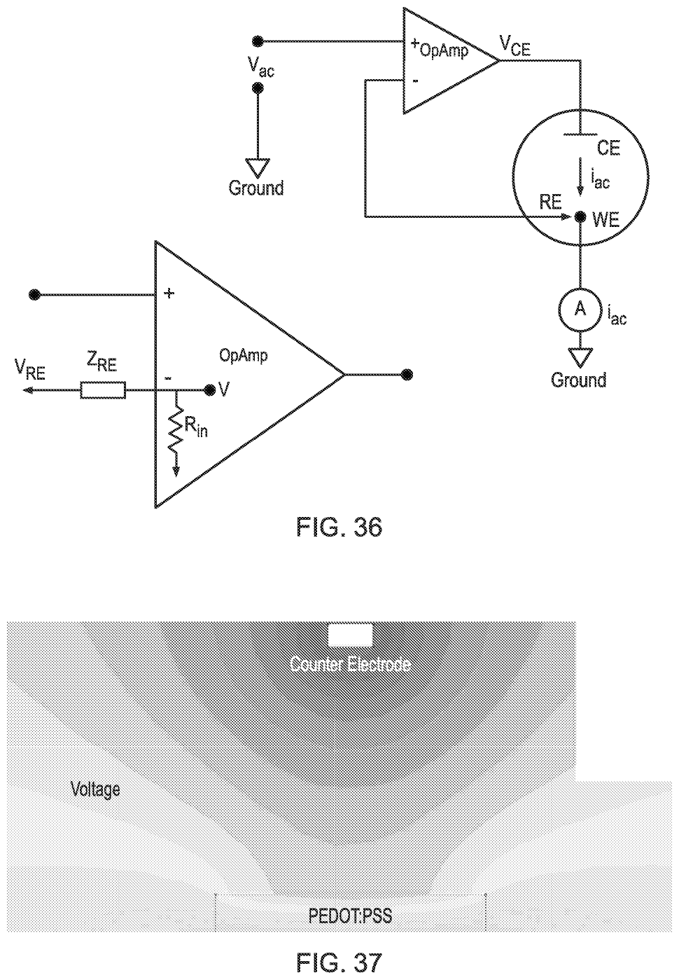

A polarizable material was proposed as the quasi-reference electrode, an important design consideration for which is derived from the potentiostat arrangement, the schematic for which is shown in FIG. 36. Potentiostats place the reference electrode in negative feedback, such that the reference electrode itself is a sense electrode, measuring the electrolyte potential. The amplifier (OpAmp) applies whatever potential to the counter electrode is required in order for the potential measured by the reference electrode to equal the specified stimulus potential. This is shown in FIG. 36a. Importantly, as is shown in FIG. 36b, when using a polarizable electrode in EIS, the electrode impedance forms a voltage divider with the input impedance of the amplifier. If the electrode impedance becomes large relative to the input impedance, then the voltage seen by the amplifier will not equal the electrolyte potential and the potentiostat function will be corrupted. Thus the reference electrode impedance, which is capacitive for polarizable electrodes, must be minimized.

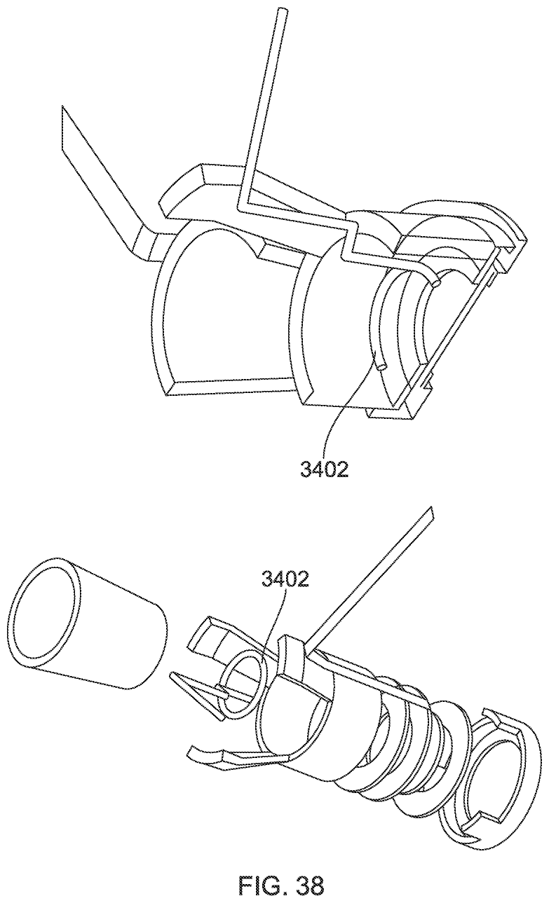

The above is achieved by maximizing the surface area of the reference electrode. However, the three dimensional potential field instantiated in such systems in highly nonuniform—thus a reference electrode which spans a large volume, may have unintended consequences on the functioning of the potentiostat. From the basic finite element simulation shown in FIG. 37, it can be seen that the equipotentials in the electrical potential field instantiated in a parallel-plate system, lie on circular contours in planes parallel to the electrode surfaces. Therefore, a ring geometry for the reference electrode allows the electrode surface area to be maximized while restricting the electrode to lie on a naturally occurring equipotential within the system. This ensures that the inclusion of the reference electrode does not alter the manner in which the device induced ionic flux in three dimensional space, while allowing the impedance of the counter electrode to be bypassed. The proposed geometry is illustrated in FIG. 35.

### Materials & Methods

### Apical Electrode

### Fabrication

The fabrication is as follows: Stainless Steel mesh (Inoxia, Woven Wire Mesh 325, 316L Stainless Steel) was cut into a rectangle, 6 mm wide and (6n+10) mm long, where n is equal to the number of folds required. The mesh strip was soldered at one end to lead wire (multistrand test lead wire, RS Components) using phosphoric acid flux (Somerset Solders, MPN: F051) and folded to yield a 6 mm×6 mm square affixed to the afore mentioned lead wire. A male Luer slip fitting (Masterflex—Item #WZ-45505-72) was press fitted into a hole drilled into the lid of a standard 24-well plate, centred above a peripheral well. The wire/mesh composite was threaded through the fitting and fixed in place with hot glue adhesive.

### Validation

The validation of the apical electrode entailed constructing two devices with identical transmembrane inserts, where one device was constructed with the conventional, platinum-based apical electrode and the other with the stainless steel-based apical electrode. The impedances in cell growth media were measured and compared.

### Polarizable Reference Electrode

### Fabrication

The reference electrode design is shown in a simplified form in FIG. 35 and in detail in FIG. 38. The design is such that the stainless steel ring (3402) is suspended above the scaffold, with the ring and scaffold planes parallel to one another. The fabrication follows below.

A 50 mm long segment of stainless steel wire (0.4 mm, 316 Grade, Scientific Wire Company) was cut and a 6 mm diameter loop formed near the end, such that the loop terminated in two wire segments, one longer and one shorter. A 5000 μl pipette tip was cut such that the inner diameter of the narrow end was [6.5-7.5] mm and the height of the cup was such that the upper lip was flush with the press-fit-legs of the cell crown, when the cup was seated into the base of the crown. Two pairs of holes were drilled into the cup, through which the two wire ends were threaded and knotted so as to fix the loop flush with the base of the cup. The cup was glued into the base of the Cell Crown (CellCrown™ inserts Z742380, Sigma Aldrich) using PDMS and the stainless steel wire not part of the ring was insulated using the same.

### Validation

The stainless steel ring as an alternative to a reference electrode in a non-Faradaic system interrogated with EIS was tested by way of a simple electrochemical cell. In particular, two strips of platinum mesh (Goodfellow, PT008710) were immersed in PBS, parallel to one another. The meshes were connected as the counter and working electrodes. Placed centrally between the meshes was either a true Ag/AgCl in 3M KCl reference electrode or a stainless steel wire ring connected as the reference electrode. The measured impedance was the interfacial impedance of the working electrode (i.e. platinum mesh). The impedances were compared.

### Validation of Complete Design

A set of devices which were constructed with the stainless steel variant of the apical electrode and the stainless steel ring reference electrode; the devices were sequentially seeded with immortalised fibroblasts, which were cultured in the scaffold for five days, at which point MDCKII cells (canine renal tubule epithelial cells) were seeded onto the apical aspect of the now colonised scaffold and cultured for a further fourteen days. On every alternating day, each device was measured first in the two electrode configuration (i.e. with the ring reference electrode left floating) and then each device was remeasured using the three electrode configuration (with the stainless steel ring connected as the reference electrode). This allowed for a direct side-by-side comparison of the reference electrode performance from the same device.

As a conservative metric for illustrating relative sensitivity, a temporal cross section through the impedance magnitudes measured at 15 Hz (a Frequency within the barrier-dominated regime) was taken for three different devices and averaged for both the two and three electrode configurations.

### Results and Discussion

In order to validate the proposed design, the performance of the conventional TxMem device with a platinum apical electrode was compared to a device with the updated stainless steel electrode. Following that, a benchtop comparison of a simple electrochemical cell using a true reference electrode was compared to the same cell, where a stainless steel wire ring was used as the reference electrode. Finally, a series of TxMem devices fabricated with the stainless steel apical electrode and stainless steel ring reference electrode were used to measure the barrier formation in a standard biological barrier model. The results from these examinations are as follows.

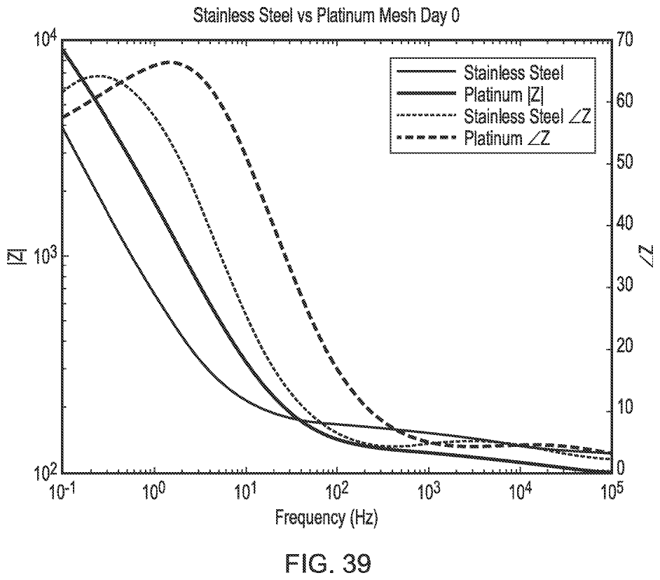

The comparison between the original and proposed apical electrodes shown in FIG. 39 indicates that both apical electrodes are strongly capacitive, with impedances dominating in the low frequency regime. Further, the stainless steel electrode can be seen to have a lower impedance magnitude and left-shifted time constant, indicative of the predicted increase in capacitance which is due to the larger surface area mesh used. Thus, the stainless steel apical electrode exhibits similar behaviour, but allows for a greater portion of the spectrum to be uncontaminated by the counter electrode impedance, thereby allowing for measurement of biological activity to occur over a greater frequency range.

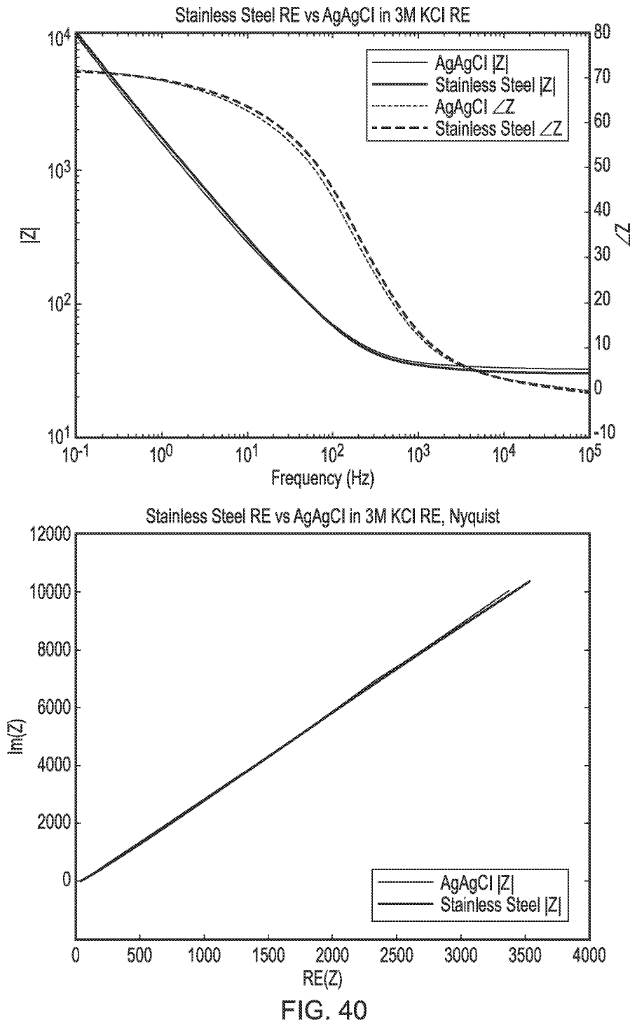

The comparison between the true reference electrode and the stainless steel wire reference electrode yielded near identical measurements of the mesh impedance, as shown in FIG. 40. In general, the discrepancy between the two is within the tolerances of the experiment (the impedance at high frequency is sensitive to relative placement of the reference electrode, which was not tightly controlled) with the exception of low frequency impedance. At the low frequency extreme, the impedance discrepancy is maximal, as seen in FIG. 40b—this is as expected, as at the lowest frequency, the stainless steel reference electrode impedance is at its largest and so the potentiostat feedback loop performs at its worst.

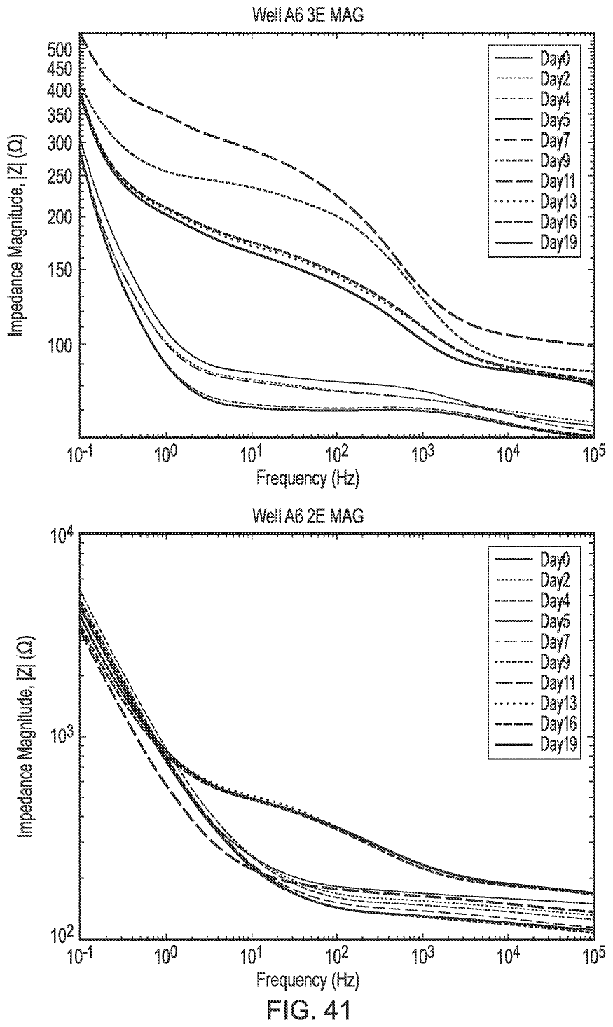

The final validation of the reference electrode system was made by observing the relative performance when measuring the formation of a barrier in a co-culture model known to reliably express a tight barrier-namely the Fibroblast/MDCKII, canine renal epithelial model. Each device was operated both in the conventional two-electrode configuration and the proposed three-electrode configuration for each measurement. The impedance magnitudes for a representative device are plotted in FIG. 41. It can be seen that the three electrode configuration (FIG. 41a) has an order of magnitude decrease in the measured impedance at low frequency, which is due to the removal of the impedance of the counter electrode by way of the reference electrode potentiostatic feedback. Indeed the mid and high frequency impedance is also decreased in the three electrode configuration, indicating that the counter electrode impedance had a non-negligible impedance even at these high frequencies, acting as a confounder in the original two electrode configuration.

Beyond the smaller impedance magnitude, the barrier dominated region (magnitude plateau) can be seen to extend for an additional order of magnitude in frequency in the three electrode configuration, illustrating how the reference electrode allows for observation of the barrier over a greater frequency range. Finally, it can clearly be seen that the magnitude at all frequencies in the three electrode configuration is temporally correlated with the formation of the barrier, indicating that information on the biological system can be extracted from all frequencies. Whereas, in the two electrode configuration, the low frequencies appear to be uncorrelated to biological events.

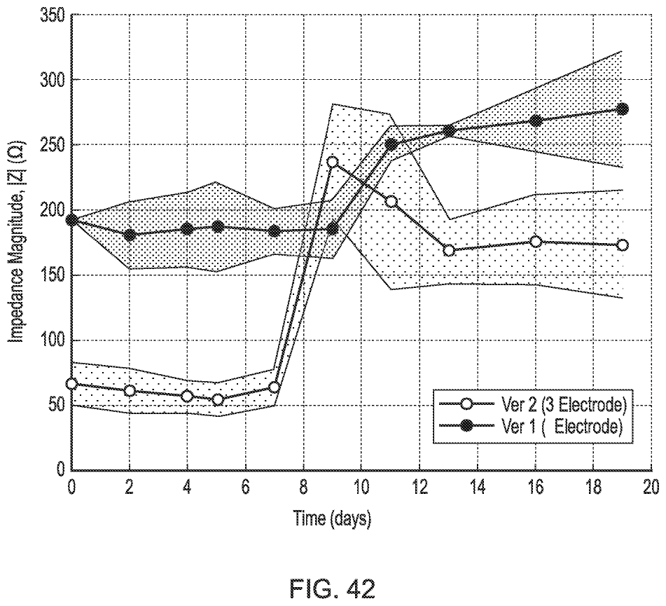

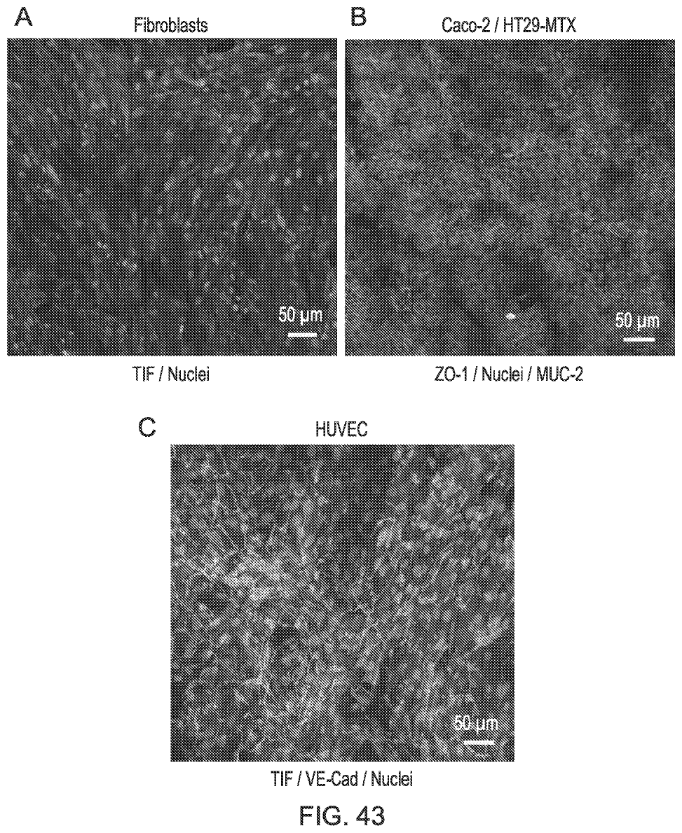

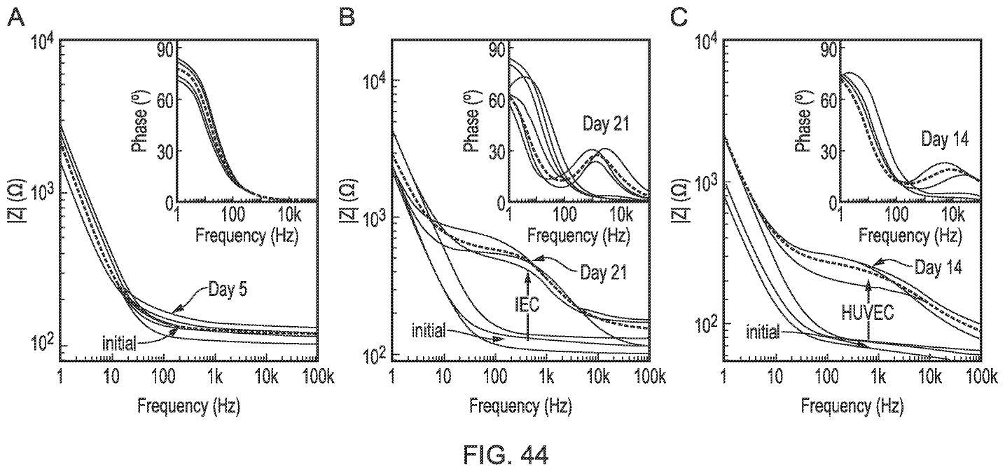

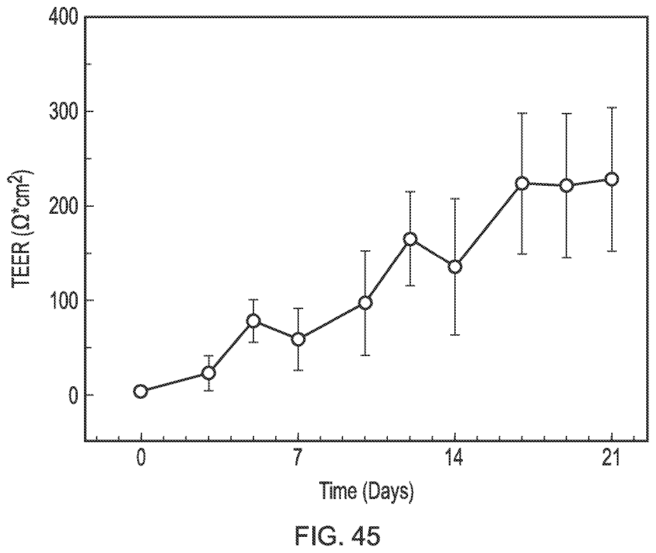

As a conservative metric, the impedance at 15 Hz is averaged across three devices in each configuration and plotted as a function of time. This plot is shown in FIG. 42. In both configurations, the barrier formation can be seen as a near discontinuity in the impedance magnitude. In the three electrode configuration, the barrier can be measured from day 7 while in the two electrode configuration the barrier can only be detected from day 9. This implies that the inclusion of the reference electrode increases the temporal sensitivity by at most one measurement cycle (two days). In addition, the three electrode configuration makes evident finer transient features of the barrier formation, with an overshooting behaviour measured. Following this overshoot, the barrier stabilizes, as expected—the measured difference in stable barrier and baseline is greater when measuring with the three electrode configuration than when using the two electrode configuration. This indicates that the inclusion of the reference electrode increases the absolute sensitivity (dynamic range) of the device as well.

The use of stainless steel mesh in the apical electrode performs at least equally well to platinum mesh, while allowing for improved stability and potentially lower counter electrode contamination (when using the two electrode configuration) by way of increases mesh surface area. Stainless steel wire has been shown to approximate a true reference electrode when conducting EIS in a non-Faradaic system; the inclusion of the wire reference electrode in the transmembrane increases both temporal and absolute sensitivity, allowing for earlier detection of barrier formation and observation of transient biological dynamics at a significantly higher resolution.

### REFERENCES

(1) Miyazaki, K.; Hashimoto, K.; Sato, M.; Watanabe, M.; Tomikawa, N.; Kanno, S.; Kawasaki, Y.; Momoi, N.; Hosoya, M. Establishment of a Method for Evaluating Endothelial Cell Injury by TNF-α in Vitro for Clarifying the Pathophysiology of Virus-Associated Acute Encephalopathy. Pediatric Research 2017, 81 (6), 942-947.
(2) Ferraretto, A.; Bottani, M.; De Luca, P.; Cornaghi, L.; Arnaboldi, F.; Maggioni, M.; Fiorilli, A.; Donetti, E. Morphofunctional Properties of a Differentiated Caco2/HT-29 Co-Culture as an in Vitro Model of Human Intestinal Epithelium. Bioscience Reports 2018, 38 (2).
(3) Shabo, I.; Midtbö, K.; Andersson, H.; Akerlund, E.; Olsson, H.; Wegman, P.; Gunnarsson, C.; Lindström, A. Macrophage Traits in Cancer Cells Are Induced by Macrophage-Cancer Cell Fusion and Cannot Be Explained by Cellular Interaction. BMC Cancer 2015, 15 (1), 1-11.
(4) Noel, G.; Baetz, N. W.; Staab, J. F.; Donowitz, M.; Kovbasnjuk, O.; Pasetti, M. F.; Zachos, N. C. A Primary Human Macrophage-Enteroid Co-Culture Model to Investigate Mucosal Gut Physiology and Host-Pathogen Interactions. Scientific Reports 2017, 7 (1), 1-14.
(5) Benson, K.; Cramer, S.; Galla, H. J. Impedance-Based Cell Monitoring: Barrier Properties and Beyond. Fluids and Barriers of the CNS. BioMed Central Jan. 10, 2013, pp 1-11.
(6) De León, S. E.; Pupovac, A.; McArthur, S. L. Three-Dimensional (3D) Cell Culture Monitoring: Opportunities and Challenges for Impedance Spectroscopy. Biotechnology and Bioengineering. John Wiley and Sons Inc. Apr. 1, 2020, pp 1230-1240.
(7) Curto, V. F.; Marchiori, B.; Hama, A.; Pappa, A.-M.; Ferro, M. P.; Braendlein, M.; Rivnay, J.; Fiocchi, M.; Malliaras, G. G.; Ramuz, M.; Owens, R. M. Organic Transistor Platform with Integrated Microfluidics for In-Line Multi-Parametric in Vitro Cell Monitoring. Microsystems & Nanoengineering 2017, 3 (April), 17028.
(8) Rivnay, J.; Inal, S.; Salleo, A.; Owens, R. M.; Berggren, M.; Malliaras, G. G. Organic Electrochemical Transistors. Nature Reviews Materials. 2018.
(9) Benson, K.; Cramer, S.; Galla, H.-J. Impedance-Based Cell Monitoring: Barrier Properties and Beyond. Fluids and Barriers of the CNS 2013, 10 (1), 5.
(10) Srinivasan, B.; Kolli, A. R.; Esch, M. B.; Abaci, H. E.; Shuler, M. L.; Hickman, J. J. TEER Measurement Techniques for In Vitro Barrier Model Systems. Journal of Laboratory Automation. SAGE Publications Inc. Apr. 1, 2015, pp 107-126.
(11) Benson, K.; Cramer, S.; Galla, H. J. Impedance-Based Cell Monitoring: Barrier Properties and Beyond. Fluids and Barriers of the CNS. BioMed Central Jan. 10, 2013, pp 1-11.
(12) Tria, S. A.; Ramuz, M.; Huerta, M.; Leleux, P.; Rivnay, J.; Jimison, L. H.; Hama, A.; Malliaras, G. G.; Owens, R. M. Dynamic Monitoring of Salmonella Typhimurium Infection of Polarized Epithelia Using Organic Transistors. Advanced Healthcare Materials 2014, 3 (7), 1053-1060.
(13) Srinivasan, B.; Kolli, A. R.; Esch, M. B.; Abaci, H. E.; Shuler, M. L.; Hickman, J. J. TEER Measurement Techniques for In Vitro Barrier Model Systems. Journal of Laboratory Automation. SAGE Publications Inc. Apr. 1, 2015, pp 107-126.
(14) van der Helm, M. W.; Henry, O. Y. F.; Bein, A.; Hamkins-Indik, T.; Cronce, M. J.; Leineweber, W. D.; Odijk, M.; van der Meer, A. D.; Segerink, L. I.; Ingber, D. E. Non-Invasive Sensing of Transepithelial Barrier Function and Tissue Differentiation in Organs-on-Chips Using Impedance Spectroscopy. Lab on a Chip 2019, 19 (3), 452-463.
(15) Curto, V. F.; Ferro, M. P.; Mariani, F.; Scavetta, E.; Owens, R. M. A Planar Impedance Sensor for 3D Spheroids. Lab on a Chip 2018, 18 (6), 933-943.
(16) Rivnay, J.; Ramuz, M.; Leleux, P.; Hama, A.; Huerta, M.; Owens, R. M. Organic Electrochemical Transistors for Cell-Based Impedance Sensing. Applied Physics Letters 2015, 106 (4).
(17) de León, S. E.; Pupovac, A.; McArthur, S. L. Three-Dimensional (3D) Cell Culture Monitoring: Opportunities and Challenges for Impedance Spectroscopy. Biotechnology and Bioengineering 2020, 117 (4), 1230-1240.
(18) Knight, E.; Przyborski, S. Advances in 3D Cell Culture Technologies Enabling Tissue-like Structures to Be Created in Vitro. Journal of Anatomy 2015, 227 (6), 746-756.
(19) Moysidou, C. M.; Barberio, C.; Owens, R. M. Advances in Engineering Human Tissue Models. Frontiers in Bioengineering and Biotechnology. Frontiers Media S.A. Jan. 28, 2021, p 620962.
(20) Ronaldson-Bouchard, K.; Vunjak-Novakovic, G. Organs-on-a-Chip: A Fast Track for Engineered Human Tissues in Drug Development. Cell Stem Cell. Cell Press Mar. 1, 2018, pp 310-324.
(21) Roth, A.; Singer, T. The Application of 3D Cell Models to Support Drug Safety Assessment: Opportunities & Challenges. Advanced Drug Delivery Reviews. Elsevier 2014, pp 179-189.
(22) Lozoya-Agullo, I.; Araújo, F.; Gonzelez-Alvarez, I.; Merino-Sanjuen, M.; Gonzelez-Alvarez, M.; Bermejo, M.; Sarmento, B. Usefulness of Caco-2/HT29-MTX and Caco-2/HT29-MTX/Raji B Coculture Models to Predict Intestinal and Colonic Permeability Compared to Caco-2 Monoculture. Molecular Pharmaceutics 2017, 14 (4), 1264-1270.
(23) Abbott, N. J.; Dolman, D. E. M.; Yusof, S. R.; Reichel, A. In Vitro Models of CNS Barriers. AAPS Advances in the Pharmaceutical Sciences Series 2014, 10, 163-197.
(24) Noonan, J.; Grassia, G.; MacRitchie, N.; Garside, P.; Guzik, T. J.; Bradshaw, A. C.; Maffia, P. A Novel Triple-Cell Two-Dimensional Model to Study Immune Vascular Interplay in Atherosclerosis. Frontiers in Immunology 2019, 10 (APR), 849.
(25) Kim, J.; Koo, B. K.; Knoblich, J. A. Human Organoids: Model Systems for Human Biology and Medicine. Nature Reviews Molecular Cell Biology. Nature Research Oct. 1, 2020, pp 571-584.
(26) Moysidou, C. M.; Owens, R. M. Advances in Modelling the Human Microbiome-Gut-Brain Axis in Vitro. Biochemical Society Transactions. Portland Press Ltd Feb. 1, 2021, pp 187-201.
(27) Nikolova, M. P.; Chavali, M. S. Recent Advances in Biomaterials for 3D Scaffolds: A Review. Bioactive Materials 2019, 4, 271-292.
(28) Moysidou, C.; Pitsalidis, C.; Al-Sharabi, M.; Withers, A. M.; Zeitler, J. A.; Owens, R. M. 3D Bioelectronic Model of the Human Intestine. Advanced Biology 2021, 5 (2), 2000306.
(29) Pitsalidis, C.; Ferro, M. P.; Iandolo, D.; Tzounis, L.; Inal, S.; Owens, R. M. Transistor in a Tube: A Route to Three-Dimensional Bioelectronics. Science Advances 2018, 4 (10), eaat4253.
(30) Jayaram, A. K.; Pitsalidis, C.; Tan, E.; Moysidou, C.-M.; de Volder, M. F. L.; Kim, J.-S.; Owens, R. M. 3D Hybrid Scaffolds Based on PEDOT:PSS/MWCNT Composites. Frontiers in Chemistry 2019, 7.
(31) del Agua, I.; Marina, S.; Pitsalidis, C.; Mantione, D.; Ferro, M.; Iandolo, D.; Sanchez-Sanchez, A.; Malliaras, G. G.; Owens, R. M.; Mecerreyes, D. Conducting Polymer Scaffolds Based on Poly(3,4-Ethylenedioxythiophene) and Xanthan Gum for Live-Cell Monitoring. ACS Omega 2018, 3 (7), 7424-7431.
(32) Inal, S.; Hama, A.; Ferro, M.; Pitsalidis, C.; Oziat, J.; Iandolo, D.; Pappa, A.-M.; Hadida, M.; Huerta, M.; Marchat, D.; Mailley, P.; Owens, R. M. Conducting Polymer Scaffolds for Hosting and Monitoring 3D Cell Culture. Advanced Biosystems 2017, 1 (6), 1700052.
(33) Guex, A. G.; Puetzer, J. L.; Armgarth, A.; Littmann, E.; Stavrinidou, E.; Giannelis, E. P.; Malliaras, G. G.; Stevens, M. M. Highly Porous Scaffolds of PEDOT:PSS for Bone Tissue Engineering. Acta Biomaterialia 2017, 62, 91-101.
(34) Moysidou, C. M.; Pitsalidis, C.; Al-Sharabi, M.; Withers, A. M.; Zeitler, J. A.; Owens, R. M. 3D Bioelectronic Model of the Human Intestine. Advanced Biology 2021, 5 (2), 2000306.
(35) Boys, A. J.; Barron, S. L.; Tilev, D.; Owens, R. M. Building Scaffolds for Tubular Tissue Engineering. Frontiers in Bioengineering and Biotechnology. Frontiers Media S.A. Dec. 10, 2020, p 589960.
(36) Wang, C.; Baker, B. M.; Chen, C. S.; Schwartz, M. A. Endothelial Cell Sensing of Flow Direction. Arteriosclerosis, Thrombosis, and Vascular Biology 2013, 33 (9), 2130-2136.
(37) Wang, K.-C.; Yeh, Y.-T.; Nguyen, P.; Limqueco, E.; Lopez, J.; Thorossian, S.; Guan, K.-L.; Li, Y.-S. J.; Chien, S. Flow-Dependent YAP/TAZ Activities Regulate Endothelial Phenotypes and Atherosclerosis.
(38) Polacheck, W. J.; Kutys, M. L.; Yang, J.; Eyckmans, J.; Wu, Y.; Vasavada, H.; Hirschi, K. K.; Chen, C. S. A Non-Canonical Notch Complex Regulates Adherens Junctions and Vascular Barrier Function. Nature 2017, 552 (7684), 258-262.
(39) Tria, S. A.; Ramuz, M.; Huerta, M.; Leleux, P.; Rivnay, J.; Jimison, L. H.; Hama, A.; Malliaras, G. G.; Owens, R. M. Dynamic Monitoring of Salmonella Typhimurium Infection of Polarized Epithelia Using Organic Transistors. Advanced Healthcare Materials 2014, 3 (7), 1053-1060.
(40) Shin, W.; Kim, H. J. Intestinal Barrier Dysfunction Orchestrates the Onset of Inflammatory Host-Microbiome Cross-Talk in a Human Gut Inflammation-on-a-Chip. Proceedings of the National Academy of Sciences of the United States of America 2018, 115 (45), E10539-E10547.
(41) Tobey, N. A.; Argote, C. M.; Hosseini, S. S.; Orlando, R. C. Calcium-Switch Technique and Junctional Permeability in Native Rabbit Esophageal Epithelium. American Journal of Physiology—Gastrointestinal and Liver Physiology 2004, 286 (6 49-6), 1042-1049.
(42) Hatayama, S.; Shimohata, T.; Amano, S.; Kido, J.; Nguyen, A. Q.; Sato, Y.; Kanda, Y.; Tentaku, A.; Fukushima, S.; Nakahashi, M.; Uebanso, T.; Mawatari, K.; Takahashi, A. Cellular Tight Junctions Prevent Effective Campylobacter Jejuni Invasion and Inflammatory Barrier Disruption Promoting Bacterial Invasion from Lateral Membrane in Polarized Intestinal Epithelial Cells. Frontiers in Cellular and Infection Microbiology 2018, 8 (JAN), 15.
(43) Tria, S.; Jimison, L.; Hama, A.; Bongo, M.; Owens, R. Sensing of EGTA Mediated Barrier Tissue Disruption with an Organic Transistor. Biosensors 2013, 3 (1), 44-57.
(44) Rothen-Rutishauser, B.; Riesen, F. K.; Braun, A.; Gunthert, M.; Wunderli-Allenspach, H. Dynamics of Tight and Adherens Junctions under EGTA Treatment. Journal of Membrane Biology 2002, 188 (2), 151-162.
(45) Ronaghan, N. J.; Shang, J.; lablokov, V.; Zaheer, R.; Colarusso, P.; Dion, S.; Désilets, A.; Leduc, R.; Turner, J. R.; Macnaughton, W. K. The Serine Protease-Mediated Increase in Intestinal Epithelial Barrier Function Is Dependent on Occludin and Requires an Intact Tight Junction. American Journal of Physiology—Gastrointestinal and Liver Physiology 2016, 311 (3), G466-G479.
(46) Samak, G.; Gangwar, R.; Meena, A. S.; Rao, R. G.; Shukla, P. K.; Manda, B.; Narayanan, D.; Jaggar, J. H.; Rao, R. K. Calcium Channels and Oxidative Stress Mediate a Synergistic Disruption of Tight Junctions by Ethanol and Acetaldehyde in Caco-2 Cell Monolayers. Scientific Reports 2016, 6 (1), 1-13.
(47) Postal, B. G.; Ghezzal, S.; Aguanno, D.; André, S.; Garbin, K.; Genser, L.; Brot-Laroche, E.; Poitou, C.; Soula, H.; Leturque, A.; Clément, K.; Carrière, V. AhR Activation Defends Gut Barrier Integrity against Damage Occurring in Obesity. Molecular Metabolism 2020, 39, 101007.
(48) Tria, S. A.; Jimison, L. H.; Hama, A.; Bongo, M.; Owens, R. M. Validation of the Organic Electrochemical Transistor for in Vitro Toxicology. Biochimica et Biophysica Acta—General Subjects 2013, 1830 (9), 4381-4390.
(49) Brunet, J.-P.; Cotte-Laffitte, J.; Linxe, C.; Quero, A.-M.; Géniteau-Legendre, M.; Servin, A. Rotavirus Infection Induces an Increase in Intracellular Calcium Concentration in Human Intestinal Epithelial Cells: Role in Microvillar Actin Alteration. Journal of Virology 2000, 74 (5), 2323-2332.
(50) Lechuga, S.; Baranwal, S.; Ivanov, A. I. Actin-Interacting Protein 1 Controls Assembly and Permeability of Intestinal Epithelial Apical Junctions. American Journal of Physiology—Gastrointestinal and Liver Physiology 2015, 308 (9), G745-G756.
(51) Carlson, T. L.; Lock, J. Y.; Carrier, R. L. Engineering the Mucus Barrier. Annual Review of Biomedical Engineering. Annual Reviews Inc. Jun. 4, 2018, pp 197-220.

1. A porous scaffold membrane configured for use in a well plate insert comprising a conducting polymer wherein the porous scaffold membrane comprises a porosity of at least about 30% and an average pore diameter from about 10 μm to about 150 μm.

2. The porous scaffold membrane according to claim 1, wherein the conducting polymer comprises poly(3,4-ethylenedioxythiophene) polystyrene sulfonate (PEDOT:PSS).

3. The porous scaffold membrane according to claim 1 or 2, wherein porous scaffold membrane is configured to allow gas and nutrient permeation.

4. The porous scaffold membrane according to any one of claims 1 to 3, wherein the porous scaffold membrane comprises a thickness from about 100 μm to 1000 μm.

5. The porous scaffold membrane according to any preceding claim, wherein the porous scaffold membrane comprises a conductivity from 5 to 100 S/cm.

6. The porous scaffold membrane according to any preceding claim, wherein the porous scaffold membrane comprises an impedance in the presence of phosphate buffered saline of between 1 and 500 Ohm at 1 kHZ and a membrane thickness of 500 μm.

7. The porous scaffold membrane according to any preceding claim, wherein the porous scaffold membrane comprises a macroporous morphology.

8. The porous scaffold membrane according to any preceding claim, wherein the porous scaffold membrane comprises a continuous structure, optionally wherein the porous scaffold membrane comprises a disc shape.

9. The porous scaffold membrane according to any preceding claim, wherein the porous scaffold membrane further comprises at least one additional agent selected from one or more of:
a. at least one cryoprotectant;
b. at least one oligosaccharide;
c. at least one polymer;
d. at least one conductivity enhancing agent;
e. at least one biopolymer; and/or
f. at least one crosslinking agent.

10. A method of producing a porous scaffold membrane according to anyone of claims 1 to 8, the method comprising:
a. forming an aqueous dispersion of a conducting polymer and poly(styrene sulfonate);
b. adding a crosslinking agent to the aqueous dispersion; and
c. lyophilising the aqueous solution.

11. The method of claim 10, further comprising prior to step c. pouring the aqueous dispersion into a mould.

12. The method according to claim 10 or 11, further comprising prior to step c., adding one or more of:
a. at least one cryoprotectant;
b. at least one oligosaccharide;
c. at least one polymer;
d. at least one conductivity enhancing agent;
e. at least one biopolymer; and/or
f. at least one further crosslinking agent,
to the aqueous dispersion.

13. A method of forming a 3-dimensional cell culture comprising the steps of:
a. seeding a porous scaffold membrane according to any one of claims 1 to 9 with a first cell type; wherein the first cell type is disposed within the porous scaffold membrane;
b. seeding at least one second cell type to at least one surface of the porous scaffold membrane; and
c. culturing the first and at least one second cell types.

14. The method of claim 13, wherein the first cell type comprises connective tissue cells and/or stroma cells; optionally wherein the first cell type comprises one or more of fibroblasts, neuronal cells and/or immune cells.

15. The method of claim 13 or 14, wherein the at least one second cell type comprises epithelial and/or endothelial cells.

16. A 3-dimensional cell culture obtainable by the method according to any one of claims 13 to 15.

17. An insert for dynamically monitoring a 3-dimensional cell culture comprising;
a. a porous scaffold membrane according to any one of claims 1 to 9;
b. at least one working electrode; and
c. a body configured to be disposed in a support a device
wherein the at least one working electrode is in electrical contact with the porous scaffold membrane.

18. The insert according to claim 17, wherein the at least one working electrode comprises a metal, optionally wherein the metal comprises gold, platinum, silver and/or lead.

19. The insert according to claim 17 or 18, wherein the at least one working electrode comprises an aperture.

20. The insert according to any one of claims 17 to 19, wherein the insert comprises a first and a second working electrode positioned on opposing surfaces of the porous scaffold membrane.

21. The insert according to any one of claims 17 to 20, wherein the insert further comprises at least one membrane support member.

22. The insert according to any one of claims 17 to 20, wherein the insert further comprises a counter electrode configured to be in electrical contact with the porous scaffold membrane and at least one working electrode via an electrolyte when in use, optionally wherein the counter electrode comprises a platinum or stainless steel mesh.

23. The insert according to any one of claim 22, wherein the counter electrode is integrated with a lid configured to cover the insert.

24. Apparatus for dynamically monitoring a 3-dimensional cell culture comprising:
a. i.) an insert according to anyone of claims 17 to 21 and a counter electrode configured to be in electrical contact with the porous scaffold membrane and at least one working electrode via an electrolyte when in use; or
ii.) an insert according to claim 22 or 23; and
b. a support device configured to receive the insert therein.

25. The apparatus according to claim 24, wherein:
the counter electrode is positioned in an upper portion of the support device; or
the insert is an insert according to claims 17 to 21 and the counter electrode is positioned in a lower portion of the support device and the at least one working electrode is located on a lower surface of the porous scaffold membrane.

26. The apparatus according to claim 24 or 25, wherein the support device further comprises a lid configured to cover the insert.

27. The apparatus according to claim 26, wherein the counter electrode is integrated with the lid.

28. The apparatus according to any one of claims 24 to 27, wherein the counter electrode comprises a platinum or stainless steel mesh.

29. The apparatus according to any one of claims 24 to 28, wherein the apparatus further comprises a reference electrode.

30. The apparatus according to any one of claims 24 to 29, wherein the support device is a well plate comprising at least one well configured to receive the insert.

31. A method of dynamically monitoring a 3-dimensional cell culture comprising the steps of:
a. providing an apparatus according to any one of claims 24 to 30;
b. forming a 3-dimensional cell culture using a method according to any one of claims 13 to 15;
c. disposing the insert into the support device;
d. immersing at least a portion of the 3-dimensional cell culture, the at least one working electrode and the counter electrode in in an electrolyte;
e. applying an electrical signal to the 3-dimensional cell culture via the counter electrode and at least one working electrode; and
f. monitoring the electrical properties of the 3-dimensional cell culture and/or the porous scaffold membrane.

32. The method according to claim 31, wherein the monitoring comprises electrical impedance spectroscopy (EIS).

33. The method according to claim 31, wherein the insert is according to claim 20 and wherein the method comprises operating the insert as a transistor.

34. The method according to claim 33 wherein, the monitoring comprises single frequency transistor-based measurements.

35. A kit of parts comprising:
an insert according to any one of claims 17 to 21, a support device according to any one of claims 24, 26 or 30 and a counter electrode according to any one of claims 24 to 28; or
an insert according to claim 22 or 23 and a support device according to claim 24 or 30.
